(19) United States
(12) Patent Application Publication
(10) Pub. No.: US 2024/0180955 A1
(43) Pub. Date: Jun. 6, 2024

(54) FIBER SUPPLEMENTATION PROTECTS FROM ANTIBIOTIC-INDUCED GUT MICROBIOME DYSBIOSIS BY MODULATING REDOX METABOLISM

(71) Applicant: Brown University, Providence, RI (US)

(72) Inventors: Peter Belenky, Providence, RI (US); Swathi Penumutchu, Providence, RI (US)

(21) Appl. No.: 18/481,330

(22) Filed: Oct. 5, 2023

Related U.S. Application Data
(60) Provisional application No. 63/378,384, filed on Oct. 5, 2022.
(60) Provisional application No. 63/477,350, filed on Dec. 27, 2022.

(51) Int. Cl. A61K 31/721; A61K 31/155; A61K 31/352; A61K 31/4412; A61K 31/4965; A61K 31/662; A61K 31/717; A61K 31/732; A61K 45/06; A61P 1/00; C12Q 1/6869; C12Q 1/689

(52) U.S. Cl. CPC A61K 31/721; A61K 31/155; A61K 31/352; A61K 31/4412; A61K 31/4965; A61K 31/662; A61K 31/717; A61K 31/732; A61K 45/06; A61P 1/00; C12Q 1/6869; C12Q 1/689; C12Q 2600/106

(57) ABSTRACT

The invention provides for the use of metformin or other mitochondrial complex 1 inhibitors with fiber probiotics to treat gut dysbiosis, especially antibiotic-induced gut dysbiosis (AID).

### CROSS-REFERENCE TO RELATED APPLICATIONS

This patent application claims priority under 35 U.S.C. § 119(e) to the provisional patent applications U.S. Ser. No. 63/378,384, filed Oct. 5, 2022, and U.S. Ser. No. 63/477,350, filed Dec. 27, 2022.

### STATEMENT REGARDING FEDERALLY SPONSORED RESEARCH OR DEVELOPMENT

This invention was made with government support under grant numbers R21 AT010366 and R01 DK125382 awarded by the National Institutes of Health. The government has certain rights in the invention.

### TECHNICAL FIELD OF THE INVENTION

This invention generally relates to drugs for disorders of the metabolism for glucose homeostasis for hyperglycemia, e.g., antidiabetics, and more particularly for use with the addition of substantially indigestible substances, e.g., dietary fibers.

### BACKGROUND OF THE INVENTION

Antibiotics are a necessary part of modern medicine, but their use often results in collateral damage to the gut microbiome. See Cabral et al. (2019); Cabral et al. (2020); Lee et al. (2020). Antibiotic-induced gut dysbiosis (AID) of native populations of bacteria in guts continues to be a clinical challenge leading to health complications such as gastrointestinal inflammation, inflammatory bowel disease, aberrant immune function, metabolic disorders (Reese et al., 2018), and colorectal cancer.

Diet has been explored to reduce antibiotic-induced gut dysbiosis. Several publications disclosed methods to decrease antibiotic stress to the microbiome using oral drug adsorbents and probiotic supplements. See de Gunzburg et al. (2018); Suez et al. (2018). These methods can create further complications by affecting drug activity on pathogens or by (in the case of probiotics) increasing gut disequilibrium.

Although diet-derived compounds such as fibers are beneficial to the gut microbiome, their mechanism of protection in antibiotic-treatment is not yet well understood. There is a need in the biomedical art to determine the mechanism of the observed phenotypes. Because of the importance of the gut microbiome to host health, protecting the gut bacterial population from antibiotic disruption is an important therapeutic target.

### SUMMARY OF THE INVENTION

The inventors discovered that the host diet can modulate the chemical environment of the gut, resulting in changes to the structure and function of the microbiome during antibiotic treatment. The inventors tested dietary fiber supplements as potential modulators of the chemical environment in the gut to reduce this pattern of dysbiosis. They used diet to change the gut chemical environment and investigate how fiber prebiotics can alleviate antibiotic-induced dysbiosis (AID) by preventing the increase in gut redox potential seen post-antibiotic treatment. In a test system of defined-diets and whole-genome sequencing of female murine microbiomes during diet modulation and antibiotic treatment, fiber prebiotics reduced the impact of antibiotic treatment on microbiome composition and function. Reduced abundance of aerobic bacteria and metabolic pathways was associated with oxidative metabolism. These metatranscriptomic results corroborated chemical measurements of eH and pH, showing that that fiber dampens the dysbiotic effects of antibiotics.

Medical professionals can now advantageously use fiber as part of a therapy for antibiotic-induced dysbiosis (AID). Using fiber in conjunction metformin or other therapeutics can modulate bacterial metabolism in the gut to prevent an increase in redox potential and protect commensal microbes during antibiotic treatment.

The invention provides for using mitochondrial complex 1 inhibitors with fiber prebiotics to treat gut dysbiosis, such as antibiotic-induced gut dysbiosis (AID). The invention provides for metformin with fiber prebiotics to treat gut dysbiosis. Other mitochondrial complex 1 inhibitors besides metformin can be used, including but not limited to rotenone, piericidin A, benzamil, phenformin, and mito-metformin or other metformin analogs developed for increased bacterial specificity. Mito-metformin is a recent variation of the drug chemically modified to have improved targeting to mitochondria. With the same reasoning, metformin variations for improved targeting to bacterial complex 1 can also be designed. Several iron chelators (ferrous or ferric), which can modulate gastrointestinal redox, including but not limited to iron chelators such as bathophenanthroline disulfonate and siderophores such as enterobactin, salmochelin, and mammalian “siderophores” such as 2,5-dihydroxybenzoic acid and lipocalin.

The invention relates to the high-energy metabolic reactions in native anaerobic gut microbes. The invention concerns host diet as a therapeutic target to modulate the structure and function of the microbiome during antibiotic treatment.

In a first embodiment, the invention provides methods of dietary fiber supplementation to alleviate antibiotic-induced gut dysbiosis when supplemented before, during, or after antibiotic treatment. Fiber prebiotics can alleviate antibiotic-induced gut dysbiosis by preventing the increase in gut redox potential seen after antibiotic treatment. Fiber supplementation during antibiotic treatment reduces bacterial respiratory activity when compared to control mice given glucose. Fiber supplementation reduces high-energy metabolic processes and promotes fermentation making this gut environment less susceptible to the oxidative damage induced by antibiotics.

Fiber supplementation promotes a gut environment that is more chemically stable and anaerobic. Supplementation is likely to reduce any gut-derived inflammation. The steps to making diet recommendations for humans is: (1) Determine approximate fiber consumption in patient to determine how likely fiber supplements will be accepted into their gastrointestinal tracts without side effects. A low baseline consumption of fiber should mean they are more sensitive to fiber and lower doses would be recommended or regimen slowly increasing dietary fiber. (2) Fiber prebiotics can usefully limit the disruption to the gut that increases complex 1 bacteria (Proteobacteria) Amoxicillin does this and other beta-lactams should have similar effects. Antibiotics and drugs can need to be assayed for their microbiome effects. When the antibiotic increases complex 1 bacteria, fiber supplementation should be beneficial. (3) Fiber prebiotics can be any plant-derived fibers, including but not limited to pectin, inulin, dextrin, levan, arabinoxylan, beta glucan, cellulose, etc.

In a second embodiment, the mitochondrial complex 1 inhibitor is metformin. The inventors used the pharmaceutical metabolic inhibitor metformin to partially mimic the effects of fiber seen during antibiotic treatment. Metformin chemically behaves as a metal chelator. Metformin can lead to significant changes in bacterial composition and can reduce spikes in oxygen-tolerant bacteria seen after antibiotic treatment. These changes to microbiome composition are time-dependent and point to more complex regulation of the microbiome by metformin that requires further exploration. Because of metformin's activity on the complex 1 protein, which is present in many phyla of bacteria, metformin can modulate metabolism in any bacteria containing mitochondrial complex 1.

In a third embodiment, the invention provides the transcriptional and chemical signatures associated with this antibiotic exposure and dietary modulation in the gut microbiome to measure changes to the microbiome during diet modulation and antibiotic treatment. The inventors used metagenomics and metatranscriptomics to measure changes to the microbiome during diet modulation and antibiotic treatment in mice. The inventors found that supplementation of fiber prebiotics alleviates the dysbiotic effect of antibiotics as measured by diversity and composition. The inventors performed metagenomic and metatranscriptomic sequencing of mouse cecal contents.

In a fourth embodiment, the invention provides a diagnostic method. The diagnostic method has the first step of sequencing the microbiome from a fecal samples, concentrating on sequencing the 16s RNA genes or sequencing metagenomic. In a particular embodiment, the patients' microbiomes are sequenced before patient treatment. This sequencing step provides a baseline microbiome that can be a control to compare to future diagnostic tests. In a second step, patients are identified as being amenable to this gut dysbiosis therapy when they have higher levels of respiratory bacteria or other complex 1 using bacteria in the gastrointestinal tract.

In a fifth embodiment, the invention provides a combined diagnostic and treatment method. patients are identified as being amenable to this gut dysbiosis therapy are treated by the treatment methods described above. In a particular embodiment, in a further step, when a patient experiences adverse gastrointestinal effects from the drug, the microbiome is reassessed to diagnosis which bacterial families are contributing to gastrointestinal distress. The efficacy of fiber supplementation can also be assessed by sequencing the microbiome and checking for abundance of inflammatory taxa such as Proteobacteria and other aerobic microbes. A positive result is that the fiber supplementation decreases Proteobacteria/aerobic bacteria in the patients' gastrointestinal tract.

In a sixth embodiment, the invention provides protection against antibiotic-induced gut dysbiosis by affecting metabolic reactions that occur at a lower redox potential. The inventors identified several important shifts in metabolic function induced by fiber supplementation that protect native gut microbes when challenged with antibiotics. The inventors corroborated these shifts in metabolic function by chemical measurements of redox potential in the gut and treatment with metformin—a potential iron chelator and metabolic inhibitor. Fiber therapeutically modulates bacterial metabolism in the gut by promoting a lower redox potential and protecting these microbes during antibiotic treatment.

In a seventh embodiment, the redox potential can be measured before during and after gut dysbiosis therapy. First, a fecal sample is collected as a baseline. To measure redox potential, a fresh sample is flash-frozen and lyophilized. The sample is then re-homogenized in reverse osmosis water and oxidation-reduction potential (ORP) electrodes are used to measure the redox potential. Then, more samples taken from patients are measured during and after treatment are compared to this sample. In an eighth embodiment, this assay can use the current method for measuring baseline or antibiotic-induced changes in redox potential in fecal samples. In another particular embodiment, the assay uses an in vivo redox sensor, which could be more accurate. Third, patients with higher baseline redox measurements are determined to be likely to benefit from gut dysbiosis therapy.

In a ninth embodiment of the invention, the assay uses qPCR to quantitate redox sensitive genes in fecal material such as superoxide dismutase, catalase and peroxidase.

In a tenth embodiment of the invention, the assay uses an ELISA or similar methodology to measure human markers of inflammation such as lipocalin.

The inventors performed electrochemical assays to show changes in the bioenergetics of the gut microbiome. The inventors tested the function of redox potential on susceptibility of gut microbes to antibiotics in vitro by inhibition of electron transport proteins, using metformin and a redox biosensor. See Liu et al. (2019). Using redox potential measurements of cecal contents, the inventors found that antibiotics lead to a large increase in gut redox potential on the glucose-fed mice and this increase is not seen in the fiber-fed mice. These electrochemical measurements further corroborate results from the sequencing data that fiber reduces antibiotic-induced gut damage. The inventors showed that metformin can inhibit bacterial metabolism and reduce redox potential protecting the native gut microbe Bacteroides thetaiotaomicron from antibiotics in vitro. They supplemented the mouse glucose-diet with metformin to explore its effects on the gut microbiome in vivo.

The inventors use supplementation of plant-derived dietary fibers to test their ability to reduce antibiotic-induced gut dysbiosis by reducing respiratory metabolism in the gut. Using a murine model and multi-omic analyses to assess the structure and function of the gut microbiome in these conditions, the inventors found that dietary fiber significantly reduces the impact of antibiotics on microbiome diversity when compared to glucose. They saw that the spike in aerobic inflammatory bacteria such as Proteobacteria is prevented by fiber supplementation. Dietary fiber changes bacterial metabolism in the gut by reducing high-redox metabolism and selecting for fermentation and autotrophic metabolism. By measuring expression of different biochemical reactions across a spectrum of electron potentials, the inventors found that fiber supplementation reduces genes involved in high-redox reactions. Bacteria with high energy electrons in their electron transport chain (ETC) have higher energetic capacity.

The inventors use the first electron transport chain complex 1 (NADH oxidoreductase), which is present in 50% of all bacteria, as a marker of gut redox potential. See Novakovsky et al. (2016); Spero et al. (2015).

In an eleventh embodiment, the invention provides for the medically useful diagnostic testing of abundance of proteobacteria/complex 1 in a stool sample. Quantitative PCR for abundance of proteobacteria is done using primers specific to Proteobacteria or specific to bacterial complex 1 subunits. The samples are tested over time to evaluate gut dysbiosis therapies throughout treatment. In a twelfth embodiment, before the prescription of a microbiome-disrupting drug, the Proteobacteria abundance is first assessed as a baseline. An increase in this abundance indicates an increased gut dysbiosis.

In a thirteenth embodiment, the invention provides methods of dietary fiber supplementation comprising the use of oats in the diet.

Using de novo assembly of metagenomic data, the inventors observe that fiber supplementation is associated with fewer metagenome assembled genomes (MAGs) containing complex 1 compared to the glucose condition post antibiotic treatment. This shows that gut redox modulation with dietary fiber can direct community composition post antibiotic-treatment. The inventors then confirmed these genetic observations using electrochemical measurements of cecal contents. They found that antibiotics significantly increased cecal redox potential in the low-fiber diet while they had a non-significant impact on the fiber diet.

Antibiotics were shown to increase redox potential in the gut. Reese et al. (2018). Here the inventors show that fiber supplementation prevents this increase in redox and reduces the ability of aerobic inflammatory bacteria to bloom during antibiotic treatment.

In vivo, the inventors showed dietary supplementation of metformin during antibiotic treatment prevents the increase in Proteobacteria in the short-term. An increase in Proteobacteria abundance indicates a more aerobic and inflammatory gut microbiome. Preventing these bacteria from expanding the gut is medically useful. This result shows modulators of high-energy metabolism can change bacterial community structure in the gut. This result provides insight into the metabolic and bioenergetic changes in the gut microbiome caused by diet and antibiotics and provides multiple therapeutic targets for decreasing antibiotic-induced gut dysbiosis.

In one aspect, this invention provides improved specificity of metformin to the gut microbiome by adding fiber prebiotics acting as drug adsorbents and a carbon source for native gut microbes. This invention advantageously improves the activity of fiber prebiotics on the gut microbiome. Dietary fiber is beneficial to the gut microbiome because it promotes native anaerobic gut microbes. See Lattimer (2010). These microbes use fermentation of fiber to make fatty acids beneficial to host health. Addition of metformin should inhibit aerobic metabolism further to promote fermentative metabolism.

Another advantages of the invention is that metformin administration is associated with negative side effects such as lactic acidosis. See Rena et al. (2017). Limiting metformin uptake with fiber prebiotics as drug adsorbents would limit these negative side effects to the host. Metformin is also associated with gastrointestinal tract distress. Providing fiber prebiotics could alleviate some of these negative gastrointestinal tract effects. See Rena et al. (2017).

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1A-1B shows that dietary fiber supplementation alleviates antibiotic-induced dysbiosis before during and after antibiotic treatment. Modified versions of the AlN-93G purified rodent diets supplemented with purified-fibers were used to modulate carbon source to the gut microbiome. The 0-fiber control received no fiber supplement, and 100% glucose was added at a 20% ratio to the diet. The fiber supplemented mice received a cocktail of seven purified-plant fibers in the ratios depicted. FIG. 1A-1B shows a mouse diet (FIG. 1A) and a mouse assay for an antibiotic intervention schematic (FIG. 1B).

FIG. 2A-2D shows that dietary fiber supplementation reduces antibiotic-induced drop in diversity and Proteobacteria. FIG. 2A is a mouse assay schematic (n=6). FIG. 2B is a set of four line graphs showing the antibiotic-induced drop in diversity Day 1 and Day 5 in glucose and fiber-supplemented mice (n=6). Statistics are by Kruskal Wallis with Dunn's Correction. Day 1: Glucose adj p value=0.0031. Day 5: Glucose adj p value=0.0049. FIG. 2C is a set of plots showing the antibiotic effect size calculated from PERMANOVA analyses of Bray-Curtis distances using the PCoA method from metagenomic and metatranscriptomic data sets (n=6). Plot displays effect size (size of the dot) with significance from adj p values denoted by significance stars. Full results are shown in source data in the Materials and Methods Section. FIG. 2D is a set of bar graphs showing changes in Bacteroides phylum at Day 1 and Day 5 of assays, adj p value=0.0049; Verrucomicrobia phylum, adj p value=0.0004; Firmicutes phylum; Proteobacteria phylum, Glucose on Day 5 versus Fiber on Day 5 adj p value=0.0014, Fiber on Day 1 versus Fiber on Day 5 adj p value=0.0478; Actinobacteria phylum, adj p value=0.0330; and Archaea, adj p value=0.0002. For FIG. 2d, (n=6). Mean±SEM. Kruskal Wallis with Dunn's Correction *p<0.05; **p<0.01; ***p<0.001; ****p<0.0001.

FIG. 3A-3C shows that dietary fiber reduces usage of oxidative metabolism and electron transport chain during antibiotic treatment: DESeq2 analysis of metatranscriptomic dataset aligned to the SEED database. Significant increases in the glucose (orange) and fiber (blue) diets on Day 1 (FIG. 3A) and Day 5 (FIG. 3B) during antibiotic treatment. Log2 FC±SEM padj<0.05 and log 2 FC>2. FIG. 3C is a schematic of proteins involved in bacterial electron transport. Significant pathways increased in fiber and glucose as determined by HUMAnN3.0 and MaAsLin2.

FIG. 4A-4D is a set of bar graphs that show that dietary fiber reduces usage of metabolic pathways with strong electron acceptors Oxygen and Nitrate: HUMaN3 reaction expression during Day 1 and Day 5 of assay. FIG. 4A shows the results for aerobic metabolism. FIG. 4B shows the results for nitrate metabolism. FIG. 4C shows the results for oxidative phosphorylation. FIG. 4D shows the results for the tricarboxylic acid cycle (TCA) cycle, also known as the Krebs cycle or citric acid cycle. Copm=copies per million reads. Kruskal Wallis with Dunn's Correction *p<0.05; **p<0.01; ***p<0.001; ****p<0.0001.

FIG. 5A-5D shows that dietary fiber reduces high redox metabolic activity and reduces abundance of complex 1 bacteria FIG. 5A shows significant differences in HUMaN3 reaction expression during Day 5 of the assay across the redox tower as determined by MaAsLin2. Coefficient on x-axis and size of dot represents q-val=FDR. Size of bubbles represents copies per million transcriptomic reads (cpm). FIG. 5B and FIG. 5C is a set of bar graphs showing change in abundance of complex 1 metagenome assembled genomes (MAGs) shown with two-tailed Mann-Whitney for significance Day 1, glucose p value=0.0043, fiber p value=0.0087 (FIG. 5B) and Day 5, glucose p value=0.0022, fiber p value=0.0260 (FIG. 5C) of the assays (n=6) *p<0.05; **p<0.01; ***p<0.001; ****p<0.0001. FIG. 5D is a bar graph (top) and a dot plot (bottom) showing eH and pH values from more mouse assays (n=6). Mean±SEM Significance determined by two-tailed Mann-Whitney test, p values left to right=0.0080, 0.0078, 0.0080 (see bar graph), with a Pourbaix diagram depicting eH and pH values from lyophilized cecal contents of mice with and without antibiotics measured within twenty-four hours after rehydration with reverse osmosis (RO) water.

FIG. 6A-6F shows that metformin protects from amoxicillin in vitro and modulates microbiome composition post-antibiotic treatment in vivo. FIG. 6A is a line graph showing NADH reporter fluorescence/bacterial growth post-antibiotic and metformin treatment. FIG. 6B is a line graph showing an amoxicillin kill curve in E. coli in response to metformin treatment. FIG. 6C is a line graph showing amoxicillin kill curve in Bacteroides thetaiotaomicron in response to metformin treatment. FIG. 6D is a schematic of mouse assay timeline. FIG. 6E is a bar graph showing metformin levels determined by mass spectrometry. FIG. 6F is a set of line graphs showing the antibiotic-induced drop in diversity Day 1 and Day 5 of assay on the glucose diet (orange) supplemented with metformin (blue). FIG. 6B and FIG. 6C significance determined by Two-Way ANOVA & Dunnett. FIG. 6F significance determined by Mann-Whitney test *p<0.05; **p<0.01; ***p<0.001; ****p<0.0001.

FIG. 7A-C is a set of graphs. FIG. 7A is a line graph showing the weight of mice throughout the assay. FIG. 7B shows the Bray-Curtis values of beta diversity during each stage of fiber supplementation. FIG. 7C is a schematic of the mouse assay timeline.

FIG. 8A-8B is a set of schematics, images, and graphs. FIG. 8A is a schematic of mouse assay and metagenomic/metatranscriptomic analyses. FIG. 8B is a set of bar graphs showing the relative abundance of bacterial phyla in short read data, including Bacterioides, Proteobacteria, Verrucomicrobia, and Firmicutes.

FIG. 9A-9B is a pair of bar graphs showing DESeq2 analysis of short read metatranscriptomic data aligned to the CAZy database showing significant increases in glucose+antibiotic (orange) and fiber+antibiotic (blue) from short read data. See FIG. 9A-9B. CAZymes related to degradation of pectin in red and inulin in green.

FIG. 10A-10F is a set of graphs and charts. FIG. 10A shows the eH values and FIG. 10B shows the pH values from lyophilized cecal contents of mice with and without antibiotics measured within ninety-six hours of rehydration with reverse osmosis (RO) water. FIG. 10C is a Pourbaix diagram depicting eH and pH. FIG. 10D shows the ATP measured from cecal contents in FIG. 10A-10C. FIG. 10E shows the ATP measured from cecal contents. FIG. 10F shows the bacterial load measured by qPCR from mice on the chow diet.

FIG. 11A-11G is a set of graphs and charts. FIG. 11A shows NADH reporter fluorescence and FIG. 11B shows bacterial growth after AB and metformin treatment. FIG. 11C shows Desulfobacterota abundance in mice on the glucose diet from FIG. 6A-6F. FIGS. 14D-14| shows data from mice on a chow diet with and without metformin/AB treatment. FIG. 11D shows metformin levels determined by mass spectrometry. FIG. 11E shows an antibiotic-induced drop in diversity Day 1 and Day 5 of assay on the unsupplemented control diet (orange) and supplemented with metformin (blue). Significance determined by Mann-Whitney test *p<0.05; **p<0.01; ***p<0.001; ****p<0.0001 FIG. 11F shows Desulfobacterota and Cyanobacteria abundance in mice on the chow diet. FIG. 11G shows the relative abundance of bacterial phyla in mouse cecums.

FIG. 12 (TABLE 1(Colon)) lists histopathology scores from blinded analysis of colon samples stained with H&E from Day 5 of the assay with glucose, glucose+antibiotic, fiber, and fiber+antibiotic. Pathologists found no significant difference.

FIG. 13 (TABLE 1(SI)) lists histopathology scores from blinded analysis of small intestine samples stained with H&E from Day 5 of the assay with glucose, glucose+antibiotic, fiber, and fiber+antibiotic. Pathologists found no significant difference.

FIG. 14 (TABLE 2) lists extended data from redox potential plot in FIG. 5A-5D with abundances of MetaCyc pathways from metatranscriptomic reads in Day 1 and Day 5 of Assay. DG values for all pathways are listed.

FIG. 15A-151 are bar graphs showing a serum cytokine panel from mouse assay. Analytes are shown in pg/mL (n=4 mice) for all cytokines assessed with two technical replicates per mouse. No significance found. Kruskal Wallis with Dunn's Correction.

FIG. 16 is a schematic showing the framework used throughout Example 11. Mice were fed a standard diet (LD01; LabDiet, St. Louis, MO, USA) for a 2-week habituation period after which they are randomly subgrouped into the four diet groups and switched to diets comprised of 80% (wt/wt) changed AlN-93G (TD.180901) base diet with 20% (wt/wt) of either pure dextrose or whole milled oats on Days −7, 0, or 5 according to the schematic. Amoxicillin challenge group mice in each diet group were administered amoxicillin on Day 0 to Day 5 and allowed to recover from amoxicillin challenge from end of Day 5 to Day 14. Fecal pellet samples were collected on Days −7, 0, 2, 4, 5, 6, 7, 9, 11, and 14. Cecum samples were obtained on Days 0, 5, and 14.

FIG. 17A-17B is a pair of bar graphs showing that whole milled oats mitigate amoxicillin-induced drop in species belonging to the Firmicutes phylum. Murine cecal content-derived metagenomic reads from the always dextrose and prophylactic oats diet groups are classified against the Mouse Gastrointestinal Bacteria Catalogue (MGBC) premade database with significantly changing species assessed by DESeq2. Results are postprocessed to exclude species with a base mean of <100, a log 2 fold change of <2, and a P value of >0.05. Bars represent mean log 2 fold change and error bars represent log fold change standard error (n=4).

### DETAILED DESCRIPTION OF THE INVENTION

### Industrial Applicability

The invention is medically useful to a broad audience, including clinicians, microbiologists, and biochemists. The specification shows the direct translational potential of dietary or pharmaceutical modulation of antibiotic-induced gut dysbiosis (AID).

The invention also provides a medically useful method to measure the gut microbiome from an ecological perspective and chemical perspective. The method shows the function that bacterial metabolism has in shaping microbiome composition.

Several publications reported that repressing microbial metabolism decreases susceptibility to antibiotics in vitro. See Belenky et al. (2015); Lopatkin et al. (2021); and Stokes et al. (2019). This susceptibility is associated with signatures of metabolic activity such as higher levels of energy carriers like ATP and NADH, higher membrane potential, and increased proton motive force. This biochemical environment is reactive, creating more radical species such as ROS and NOS when compared to a bacterium with repressed metabolism. Elevated pH, uncoupling electron transport and decreasing glucose availability were all shown to suppress microbial metabolism and protect from antibiotics. See Dwyer et al. (2014); Lopatkin et al. (2021).

The invention provides that modulating the metabolism of gut bacteria in the host is now a potential therapeutic target for antibiotic-induced gut dysbiosis.

Gut resident bacteria rely on the host's diet for metabolic inputs. Host diet is an important factor shaping the chemical environment of the gut as well as the structure and function of the microbiome. Ng et al. (2019); Yang et al., (2020). The type of carbon source in the diet can determine which electron acceptors reach bacteria in the gut driving specific and predictable biochemical reactions. Miller et al. (2021). Simple carbon sources present in the Western high-sugar diet are quickly absorbed by the host, limiting carbon for microbes in the gut. These gut microbes compete for the limited carbon available. These microbes metabolize host-derived carbon from mucosal linings in the intestine. Earle et al. (2015); Fan & Pedersen (2021). This metabolism increases gut inflammation and changes the structure of the microbiome by selecting for bacteria that thrive in this inflammatory and aerobic environment. Rivera-Chávez et al. (2017).

This aerobic environment provides the strong electron acceptor. Oxygen thermodynamically selecting for metabolic reactions with higher redox potential energy. Miller et al. (2021); Khademian & Imlay (2021).

Providing complex carbon sources such as fiber prebiotics to the host increases the carbon pool to gut microbes because fiber is poorly digested by the host. Dietary fiber selects for microbes that can metabolize complex polysaccharides using fermentative metabolism. Short chain fatty acids (SCFAs) are produced by bacteria during fiber fermentation. Short chain fatty acids are then metabolized by colonocytes in an oxygen consuming reaction. Kim et al. (2018); Litvak et al. (2018); So et al. (2018) This metabolism creates improved intestinal integrity and increases the anaerobicity of the gut. Metabolic reactions with lower redox potential energy are thermodynamically favored, such as fermentation. Low-fiber diets increase oxygen and strong electron acceptors, favoring metabolic reactions with high redox potential, while fiber-rich diets reduce oxygen and select for low redox potential reactions. See Miller et al. (2021).

Diet should be able to be a modulator of the chemical redox environment in the gut and select for specific biochemical reactions that reduce or elevate microbial susceptibility to antibiotics. Several publications described the function of host diet on antibiotic-induced gut dysbiosis in vitro. Diet-derived fibers such as xanthan gum protected the drop in bacterial diversity seen post-antibiotic treatment. See Schnizlein et al., (2020). A high-fat high-sugar Western style diet can exacerbate antibiotic-induced gut dysbiosis. See Obrenovich et al. (2020); Zake et al. (2021); Cabral et al. (2020); Lee et al. (2020).

### Definitions

For convenience, the meaning of some terms and phrases used in the specification, examples, and claims are listed below. Unless stated otherwise or implicit from context, these terms and phrases shall have the meanings below. These definitions aid in describing particular embodiments but are not intended to limit the claimed invention. Unless otherwise defined, all technical and scientific terms have the same meaning as commonly understood by a person having ordinary skill in the art to which this invention belongs. A term's meaning provided in this specification shall prevail if any apparent discrepancy arises between the meaning of a definition provided in this specification and the term's use in the biomedical art.

Antibiotic-induced dysbiosis has the biomedical art-recognized meaning. See Pickard, Zeng, Caruso & Núñez, Gut microbiota: Role in pathogen colonization, immune responses, and inflammatory disease. Immunol. Rev., 279, 70-89 (2017).

Antibiotic has the biomedical art-recognized meaning of a therapeutic agent used to reduce a biotic, e.g., bacterial, infection. Antibiotic tetracycline compound has the biomedical art-recognized meaning of compound having antibiotic activity within the class of which tetracycline is the parent compound and is characterized by a unique four-ring structure. Amoxicillin does this and other beta-lactams should have similar effects.

Complex 1 has the biomedical art-recognized meaning. Respiratory complex I, EC 7.1.1.2 (also known as NADH: ubiquinone oxidoreductase, Type I NADH dehydrogenase and mitochondrial complex I) is the first large protein complex of the respiratory chains of many organisms. Complex 1 catalyzes the transfer of electrons from NADH to coenzyme Q10 (CoQ10) and translocates protons across the inner mitochondrial membrane in eukaryotes or the plasma membrane of bacteria. Complex 1 can be a sign of the bioenergetic capacity. Complex 1 has one of the largest iron-sulfur clusters in bacteria making this protein responsible for high-energy electron transfers.

Complex 1 inhibitors has the biomedical art-recognized meaning. Complex 1 inhibitors include, but are not limited to metformin, rotenone, piericidin A, benzamil, phenformin, and metformin, mito-metformin or other analogs developed for increased bacterial specificity. Multiple iron chelators (ferrous or ferric), which can modulate gastrointestinal redox including bathophenanthroline disulfonate, and siderophores such as enterobactin, salmochelin, and mammalian “siderophores” such as 2,5-dihydroxybenzoic acid and lipocalin.

Dysbiosis has the biomedical art-recognized meaning. Dysbiosis is characterized by a decrease in microbial diversity and increase in proinflammatory species. This imbalanced microbiota cannot protect from pathogenic organisms, that can trigger inflammation and produce genotoxins or carcinogenic metabolites. Gut dysbiosis is characterized by increases in aerobic respiratory bacterial metabolism, redox potential, and abundance of Proteobacteria.

Fiber in this patent specification has the meaning of dietary fiber, including but not limited to plant-derived polysaccharides indigestible by human-derived carbohydrate enzymes.

Gut microbiome has the biomedical art-recognized meaning. The gut microbiome has little Proteobacteria when in a healthy state with low gut inflammation. The abundance of Proteobacteria/complex 1 bacteria can be a sign of gut inflammation because they are largely aerobic with active metabolisms. The metabolic capacity of Proteobacteria/complex1 bacteria lets them rapidly expand in an inflammatory environment where they can use the strong electron acceptor oxygen. Diet is an important factor shaping the gut microbiome.

Metabolism has the biomedical art-recognized meaning and encompasses both microbial metabolism and host metabolism. Previous in vitro and in vivo publications identified that a change in bacterial metabolism can promote tolerance or susceptibility to antibiotics. Active metabolism is associated with increased susceptibility while metabolic dormancy confers protection. See Cabral et al. (2019); Belenky et al. (2015); and Stokes et al. (2019).

Metformin has the biomedical art-recognized meaning. Metformin is a pharmaceutical inhibitor of complex I and other iron-related enzymes. See Fontaine (2018); Rena et al. (2017). Metformin reduces the NADH/NAD⁺ ratio. Liu et al. (2019). Metformin is of the class of drugs called biguanides, which inhibit the production of glucose in the liver. Metformin is sold under the trade names Glucophage®, Fortamet®, Glumetza®, and Riomet®. Metformin has been an anti-diabetic/anti-obesogenic drug for the past sixty years. See Rena et al. (2017).

Microbiome has the biomedical art-recognized meaning of a community of bacteria, viruses, fungi present in multiple environments including human associated such as the gut but can also include microbial communities in the soil, water and anywhere else complex communities of microbes form.

Redox has the biomedical art-recognized meaning. Redox potential (eH) is the measure of electron movement and electron potential energy in a system. Redox potential can show the amount of energy being transferred in biochemical reactions.

Subject or Host has the biomedical art-recognized meaning of an animal, e.g., a mammal, e.g., a human to whom a treatment could be administered. A subject can be a patient. An animal host has commensal microbes in the host gut.

Unless otherwise defined, scientific and technical terms used with this application shall have the meanings commonly understood by persons having ordinary skill in the biomedical art. This invention is not limited to the particular method, protocols, reagents, etc., described herein and can vary.

This specification does not concern a process for cloning humans, methods for modifying the germ line genetic identity of humans, uses of human embryos for industrial or commercial purposes, or procedures for modifying the genetic identity of animals likely to cause them suffering with no substantial medical benefit to man or animal resulting from such processes.

Guidance from Materials and Methods

A person of ordinary skill in the biotechnological art or the oncological art can use these materials and methods as guidance to predictable results when making and using the invention:

Mice. Assay procedures involving mice were all approved by the Institutional Animal Care and Use Committee of Brown University. Four-week-old male C57BL/6J mice were purchased from the Jackson Laboratories (Bar Harbor, ME, USA). Mice were habituated for two-weeks following their arrival at Brown University. All animals were cohoused together in specific-pathogen-free (SPF), temperature controlled (21+1.1° C.), and 12-hour light/dark cycling conditions within Brown University's animal care facility. Mice were randomized into new cages following the habituation period. Mice were given the specified diets in powdered form. Mice used for redox potential measurements were given the typical laboratory chow (Laboratory Rodent Diet 5001, LabDiet, St. Louis, MO, USA).

In Example 11, animal procedures were approved by the Brown University Institutional Animal Care and Use Committee under protocol 20-06-0001. Seventy-two five-week-old female C57BL/6J mice were obtained from The Jackson Laboratory (Bar Harbor, ME, USA) and housed in Brown University's specific-pathogen-free Animal Facility.

Bacterial strains. Bacteroides thetaiotaomicron (VPI-5482) can be obtained from the American Type Culture Collection (ATCC; Manassas, VA, USA). Bacteroides theta was cultured in modified Gifu Anaerobic Media (mGAM, HyServe). Escherichia coli (MG1655) was cultured in Luria Broth (Fisher Scientific, Waltham, MA, USA). Anaerobic assays were cultured in a BactronEZ anaerobic chamber (Sheldon Manufacturing, Cornelius, OR, USA) under an atmosphere of 5% H₂, 5% N₂, and 90% CO₂. All bacteria were grown at 37° C.

Diet design. Purified diets were designed with veterinarians from Envigo-Teklad (Madison, WI, USA) based on fiber content present in the typical mouse laboratory chow. The diet is based on the purified AlN-93G (TD. 180901) diet and changed to have reduced carbohydrates with the cellulose removed and the cornstarch reduced. The cornstarch supplied in the diet (Buffalo cornstarch, Envigo-Teklad) is changed to be more host-accessible reducing its prevalence in the cecum and lower gastrointestinal tract. This diet was custom designed at 80% composition to allow for 20% (w/w) supplementation with other carbon sources without affecting protein, fat, vitamin and mineral ratios. The diet is powdered and irradiated and given to mice in feeding jars. Mice were allotted 5 g/mouse/day in the jars and the food was replenished daily in new autoclaved feeding jars. This quantity was determined after conversations with Envigo-Teklad veterinarians. This quantity is well over the typical amount consumed by mice. The glucose diet was used a no fiber control in all assays and supplemented with 20% glucose (Fisher Scientific). The fiber diet has 20% (w/w) supplementation of a custom fiber cocktail including inulin (15%) (Chem-Impex), pectin (15%) (MP Biomedicals), dextrin (15%) (Sigma-Aldrich), levan (15%) (Realbiotech Co., Ltd.), arabinoxylan (20%) (Anthony's Organics), beta-glucan (25%) (Anthony's Organics), cellulose (10%) (EMD Millipore). For single purified fiber supplementation, a 95% composition of the same diet was used with 5% (w/w) supplementation of pectin or inulin.

In Example 11, mice were fed LabDiet 5001 (LD01; LabDiet, St. Louis, MO, USA) feed pellets for a 2-week habituation period. For experimental diets, a custom base diet reflecting standard mouse chow fiber content was designed (Envigo-Teklad, Madison, WI, USA). The base diet modifies AlN-93G (TD.180901) by removing all cellulose and reducing cornstarch content while adjusting micro- and macronutrient contents so the diet could be an 80% base for 20% (wt/wt) supplementation with other carbon sources. Buffalo cornstarch (Ingredion Incorporated, IL, USA) was used to increase host accessibility and reduce cornstarch availability in the cecum and lower gastrointestinal tract. The dextrose diet was a powdered diet of 80% base and 20% glucose (Fisher Scientific, Waltham, MA, USA). The oats diet was a powdered diet of 80% base diet and 20% whole milled oats. Whole milled oats were prepared by liquid nitrogen snap-freezing and complete milling followed by baking at 120° C. for twenty minutes. Five grams per mouse of irradiated powdered diets were provided to mice daily to consume ad libitum.

Mouse assays. C57BL/6J mice following the habituation period were given one week to acclimate to the control 0-fiber diet with 20% (w/w) glucose. After this diet acclimation period, mice were randomized into new cages for each diet/antibiotic condition. Amoxicillin was administered via drinking water (25 mg/kg/day) ad libitum for the specified timepoints. All drinking water was filter-sterilized before administration. Fecal samples were collected at the specified timepoints and stored at −20° C. until nucleic acid extraction. Cecum contents were collected directly into bead-bashing tubes with DNA/RNA shield (Zymo Research, Irvine, CA, USA) and stored at −80° C. for nucleic acid extraction. For the redox potential (eH) and pH measurements, total cecal contents were immediately flash frozen in liquid nitrogen and lyophilized and stored at −80° C. until measurements were taken.

In Example 11, following habituation, mice were grouped randomly and then acclimated for one week on either the dextrose or oats diet. Mice were then grouped into cages according to continuing a dextrose (always dextrose group) or oats (always oats group) diet or switching from dextrose to oats (prophylactic oats group). Groups where subdivided into control or amoxicillin-treated cages and provided ad libitum filter-sterilized water or filter-sterilized water with 0.1667 mg/mL amoxicillin. Treatment cages were provided fresh amoxicillin water bottles daily. Amoxicillin challenge course lasted five days. After treatment, a subgroup of the always dextrose group was switched from the dextrose to the oats diet (recovery oats group). Mice then recovered for 9 days until experiment completion. Fecal samples were collected and stored in DNA/RNA shield (Zymo Research; Irvine, CA, USA) at −80° C. on days −7, 0, 2, 4, 5, 6, 7, 9, 11, and 14. Cecum samples were collected in bead-bashing tubes with DNA/RNA shield and stored at −80° C. on days 0, 5, and 14.

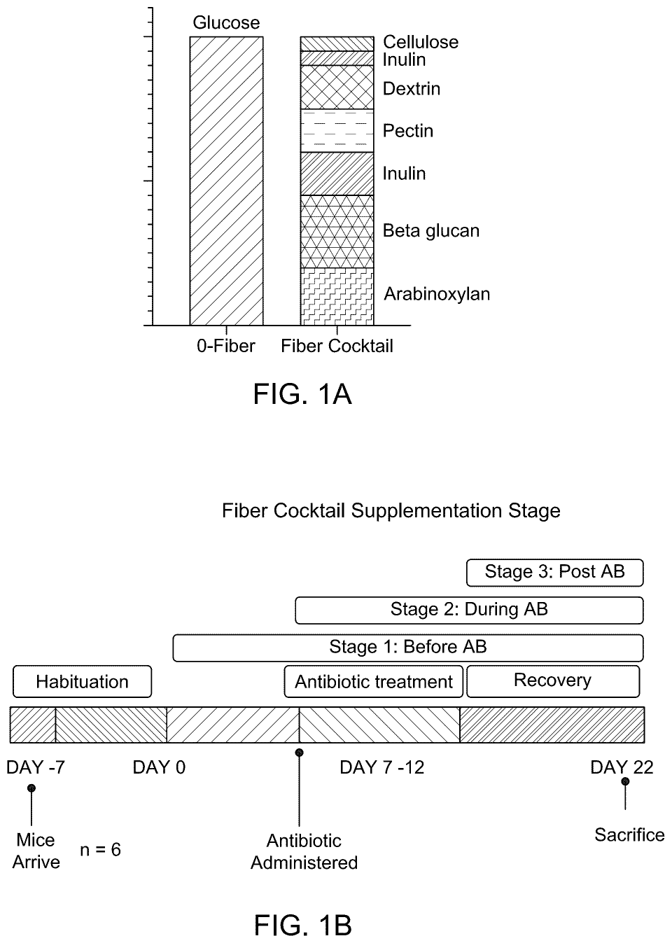

Mouse assay setup: Female C57BL/6 mice can test the effects of purified-plant fiber supplementation on antibiotic-induced gut dysbiosis in mice fed the purified AlN-93G diet (Envigo-Teklad). The mouse diet is prepared at 80% composition, allowing for 20% supplementation of a carbon source while controlling for fat, protein and trace vitamin and mineral concentrations between diet groups. The inventors used glucose as the control low-fiber unsupplemented condition, and a cocktail of seven plant fibers including cellulose, levan, dextrin, pectin, inulin, beta-glucan, and arabinoxylan for the fiber-supplemented conditions. See FIG. 1A. Fiber, plant-derived carbohydrates that can by digested by human digestive enzymes, can include other fiber, such as xyloglucans, gums (guar and xanthan), psyllium, pullulan, other dextrins and beta-glucans.

Mice were given two weeks to habituate in the facility and then split into eight groups (n=6) supplemented with the fiber cocktail before, during or after antibiotic treatment. See FIG. 1B. Antibiotics were administered (amoxicillin 150 mg/L) via drinking water for five days (Day 7-12 post-habituation) while the No Treatment received normal drinking water.

Nucleic acid extraction and measurement. Total nucleic acids (DNA and RNA) were extracted from samples using the ZymoBIOMICS DNA Miniprep Kits from Zymo Research (Irvine, CA, USA) using the extraction protocols as per the manufacturer instructions. For fecal samples the Fecal 96 Zymo DNA Extraction kit was used. For DNA/RNA parallel extraction the Zymo Magbead DNA/RNA kit was used. Total DNA was eluted in nuclease-free water and measured using the dsDNA-HS on a Qubit™ 3.0 fluorometer (Thermo Fisher Scientific, Waltham, MA, USA) before use in amplicon/library preparations.

In Example 11, total DNA and RNA was liberated from bacteria by bead-bashing fecal or cecum samples for 5 min on a Bead Ruptor 96 (Omni International, Kennesaw, GA). Fecal DNA was extracted according to the manufacturer's instructions using the Fecal/Soil Microbe 96 MagBead kit (D6011-FM, Irvine, CA, USA). Total DNA and RNA were coextracted and isolated from cecum samples according to the manufacturer's instructions using the ZymoBIOMICS MagBead DNA/RNA kit (R2136). Nucleic acids were eluted in nuclease-free water and measured using the double-stranded DNA high sensitivity (dsDNA-HS) or RNA-HS kits on a Qubit 3.0 fluorometer (Thermo Fisher Scientific, Waltham, MA, USA).

16S rRNA amplicon preparation and sequencing. The 16S rRNA V4 hypervariable region was amplified from total DNA using the barcoded 515F forward primer and the 806R reverse primers from the Earth Microbiome Project (Thompson et al., 2017). Amplicons were generated using 5× Phusion High-Fidelity DNA Polymerase under these cycling conditions: (1) initial denaturation at 98ºC for thirty seconds, followed by (2) twenty-five cycles of 98ºC for ten seconds, 57ºC for thirty seconds, and 72° C. for thirty seconds, then (3) a final extension at 72ºC for five minutes. Gel electrophoresis was used to visualize amplicons and pooled in equimolar amounts. The pooled amplicon library was submitted to the Rhode Island Genomics and Sequencing Center at the University of Rhode Island (Kingston, RI, USA) for sequencing on the Illumina MiSeq platform. Amplicons were paired-end sequenced (2×250 bp) using the 600-cycle kit with standard protocols. Raw reads were deposited in the NCBI Sequence Read Archive (SRA).

In Example 11, the 16S rRNA gene V4 region was amplified from fecal total DNA using barcoded 515F forward and indexed 806R reverse primers as described by Parada, Needham, & Fuhrman, Environ. Microbiol. 18, 1403-1414 (2016). Amplicons were generated with Phusion high-fidelity DNA polymerase (F530L; Thermo Fisher) following the program earth microbiome protocol. See Parada, Needham, & Fuhrman, Environ. Microbiol. 18, 1403-1414 (2016). Amplicons were verified by gel electrophoresis, measured on the Qubit 3.0 and pooled in equimolar concentrations. Amplicons were paired-end (2×250 bp) sequenced by Illumina MiSeq sequencing using the 600-cycle kit according to manufacturer's protocols at the Rhode Island Genomics and Sequencing Center at the University of Rhode Island. The average read depth was 31,737 (+15,392) reads per sample.

16S sequencing analysis. Raw 16S rRNA reads were first demultiplexed with idemp. Quality filtering, trimming, de-noising with DADA2 (q2-dada2). See Callahan et al. (2016). Merging was done using the Qiime2 pipeline (version 2019.10). See Bolyen et al., (2019). Ribosomal sequence variants were aligned with mafft (q2-alignment). See Katoh et al. (2002). Phylogenetic tree construction was done with fasttree2 (q2-phylogeny). See Price et al. (2010). Taxonomic assignment was conducted using the pre-trained Naive Bayes classifier and the q2-feature-classifier (Bokulich et al., 2018) trained on the SILVA 132 99% database. Quast et al. (2013). Alpha diversity (Shannon, Faith's phylogenetic diversity) and beta diversity (Bray-Curtis dissimilarity) were calculated using the phyloseq package (version 1.30.0) in R (version 3.6.2). See Bokulich et al. (2018); Lozupone & Knight (2005); McMurdie & Holmes (2013).

In Example 11, 16S rRNA gene sequences were demultiplexed with idemp, see Wu, Idemp (2017). https://github.com/yhwu/idemp; quality filtered; trimmed; and denoised with the DADA2. See Callahan et al., DADA2: High-resolution sample inference from Illumina amplicon data. Nature Methods, 13, 581-583 (2016). QIIME 2 plugin (q2-dada2) and then merged with QIIME 2 (version 2022.8). Bolyen et al., Nature Biotechnol., 37, 852-857 (2019). Sequence variants were aligned. A phylogenetic tree derived with MAFFT (q2-alignment), Katoh, Misawa, Kuma, & Miyata, Nucleic Acids Research, 30, 3059-3066 (2002), and FastTree 2 (q2-phylogeny), Price, Dehal, & Arkin, PLOS One, 5, e9490 (2010). Taxonomic information was assigned through a pretrained naïve Bayes classifier (q2-feature-classifier), Bokulich et al, Microbiome, 6, 90 (2018), trained on the SILVA 132 99% database, Quast et al., Nucleic Acids Research, 41, D590-D596 (2013). Taxonomic diversity was assessed by alpha (Shannon & Faith's phylogenetic diversity) and beta diversity (Bray-Curtis dissimilarity) metrics through the phyloseq package (version 1.38.0) in R (version 4.1.3). See McMurdie & Holmes, phyloseq: An R package for reproducible interactive analysis and graphics of microbiome census data. PLOS One, 8, e61217 (2013); Lozupone & Knight, UniFrac: A new phylogenetic method for comparing microbial communities. Appl. Environ. MicroBiology, 71, 8228-8235 (2005).

Metagenomic and metatranscriptomic library preparation. Metagenomic and metatranscriptomic sequencing libraries were prepared as described by Cabral (2020). For metagenomic libraries 100 ng of DNA was used with the NEBNext® Ultra II FS DNA Library Prep Kit (New England BioLabs, Ipswich, MA, USA) as per manufacturer's instructions to generate a pool of fragments at 300 bp±50 bp. Metatranscriptomic libraries were created with total RNA (1 μg) using the MICROBExpress kit (Invitrogen, Carlsbad, CA, USA), NEBNext® rRNA Depletion Kit for Human/Mouse/Rat (New England BioLabs, Ipswich, MA, USA), and the NEBNext® Ultra II Direction RNA Sequencing Prep Kit as per the manufacturers' instructions to generate a pool of fragments at 300 bp□50 bp. Metagenomic and metatranscriptomic libraries were pair-end sequenced (2×150 bp) on the Illumina HiSeq X Ten. An average of 12,928,385 reads per metagenomic sample and 52,848,755 reads per metatranscriptomic sample. A control metagenomic sequencing library was made using the Zymobiomics Microbial Community Standard (D6300, Zymo Research, Irvine, CA USA). The control sequence was added to the sequencing run. This data was used to confirm the accuracy of the sequencing run.

Sequencing of this standard resulted in relative abundances near the theoretical composition with all community members identified.

In Example 11, metagenomic libraries were generated from 100 ng of DNA using the NEBNext Ultra II FS DNA library prep kit (E7805L; New England BioLabs, Ipswich, MA, USA) with ≥100 ng input protocol per manufacturer's instructions. Pool fragments ranged from 250 to 1,000 bp averaging to ˜400 bp. Metatranscriptomic libraries were prepared with ˜1 μg of total RNA using the NEBNext Ultra II directional RNA sequencing prep kit (E7760S) with the NEBNext rRNA depletion kit for human/mouse/rat (E6310L) and the MICROBExpress kit (AM1905; Invitrogen, Carlsbad, CA, USA) according to manufacturer's instructions. Pool fragments ranged from 200 to 500 bp averaging to ˜275 bp. Metagenomic and metatranscriptomic libraries were pair-end sequenced (PE150) on the NovaSeq 6000 (Novogene, Sacramento, CA, USA). Metagenomic samples averaged 35,780,052 (±20,198,450) reads per sample and metatranscriptomic samples averaged 152,707,388 (±33,779,684) reads per sample.

Metagenome assembly. Metagenome-assembled genomes from the metagenomic reads were constructed using the metaWRAP pipeline. See Uritskiy et al. (2018). Raw reads were processed using the READ_QC module with FASTQC and TrmGalore. Assembly was done with the ASSEMBLE module in metaWRAP with metaSPAdes (Nurk et al. (2017)) and MegaHit (Li et al. (2015)). Assembled contigs were binned with the BINNING module using CONCOCT, MaxBin2 (Wu et al. (2016)) and metaBAT2. See Kang et al. (2019). The BIN_REFINEMENT and BIN_REASSEMBLY module was used to consolidate bins and select bins with greater than 90% completion and fewer than 10% contamination to reach fifty-four high-quality metagenome assembled genomes. Measurement was done using the QUANT_BINS module with SALMON. See Patro et al. (2017). Measurement was classified with the CLASSIFY_BINS module and taxator-tk. The CAT and BAT tool was also used. See von Meijenfeldt et al. (2019).

MAGs phylogenetic tree construction and complex 1 identification. Phylophlan 3.0 was used to construct a phylogenetic tree from the assembled metagenome assembled genomes using the high diversity option and the phylophlan database. To identify metagenome assembled genomes with complex 1, a custom phylophlan database was constructed using the Uniref90 sequence clusters for each of the fourteen subunits (A-N) of the bacterial NADH-quinone oxidoreductase. TABLE 2 has the Cluster ID and size information. Phylophlan was then run using this database to construct a phylogenetic tree of metagenome assembled genomes to identify complex 1 MAGs.

Metagenomic and metatranscriptomic short read processing. Raw metagenomic and metatranscriptomic reads underwent trimming and decontamination using KneadData (version 0.6.1) as described by Beghini et al. (2021); Cabral et al. (2019); Cabral et al. (2020). Illumina adaptor sequences were removed using Trimmomatic (version 0.36). See Bolger et al. (2014). Reads that mapped to C57BL/6J, murine mammary tumor virus (MMTV, accession NC_001503) and murine osteosarcoma virus (MOV, accession NC_001506.1) were depleted using Bowtie2 (version 2.2). See Cabral et al. (2019); Langmead & Salzberg (2012). Metatranscriptomic reads were also depleted of sequences that aligned to the SILVA 128 LSU and SSU Parc ribosomal RNA databases as described by Cabral et al. (2019); Cabral et al. (2020); Quast et al. (2013)

Short read classification. Classification of metagenomic reads was done with NCBI RefSeq using Kraken2 (version 2.0.7-beta, “Kraken2 Standard Database”) with a k-mer length of 35 (Wood et al., 2019). Bracken (version 2.0.0) was then used to calculate phylum-level and species-level abundances from Kraken2 reports. The R package phyloseq (version 1.28.0) was used to calculate a-diversity and b-diversity metrics. See McMurdie & Holmes (2013).

Metagenomic and metatranscriptomic short-read processing. In Example 11, raw metagenomic and metatranscriptomic reads were trimmed and decontaminated with Trimmomatic (version 0.39), Bolger, Lohse, & Usadel, Bioinformatics, 30, 2114-2120 (2014), and KneadData (version 0.6.1), McIver et al., Bioinformatics, 34, 1235-1237 (2018). Trimmomatic removed low-quality reads and Illumina TruSeq3 adapter sequences with the SLIDINGWINDOW value 4:20, ILLUMINACLIP value 2:20:10, and MINLEN value 75. Quality controlled reads were decontaminated using bowtie2 (version 2.2), Langmead & Salzberg, Nature Methods 9, 357-359 (2012), removing reads mapping to the C57BL/6J mouse genome or two murine retroviruses found in the animal facility: murine mammary tumor virus (MMTV) (GenBank accession number NC_001503) and murine osteosarcoma virus (MOV) (GenBank accession number NC_001506.1). Raw metatranscriptomic reads were processed similarly except for an additional decontamination of sequences aligning with the SILVA 128 LSU and SSU Par crRNA databases. See Pruesse et al., SILVA: A comprehensive online resource for quality checked and aligned ribosomal RNA sequence data compatible with ARB. Nucleic Acids Research, 35, 7188-7196 (2017).

Metagenomic short-read classification. In Example 11, metagenomic reads were classified against the pre-built Mouse Gastrointestinal Bacteria Catalogue (MGBC) Kraken 2/Bracken database, see Beresford-Jones et al., Cell Host Microbe, 30, 124-138 (2022), using Kraken 2 (version 2.0.7-beta) with default k-mer and I-mer values. Wood, Lu & Langmead, Genome Biology, 20, 257 (2019). Phylum and species abundances were calculated with Bracken (version 2.7.0) using Kraken 2 reports. Lu, Breitwieser, Thielen, & Salzberg, PeerJ Comput., Sci 3, e104 (2017).

Metatranscriptomic analysis: SAMSA2. A modified version of the Simple Annotation of Metatranscriptomes by Sequences Analysis 2 (SAMSA2) pipeline to annotate trimmed and decontaminated reads as described by Cabral et al. (2019); Westreich et al. (2018); Wurster et al. (2021). This modified pipeline uses Paired-End Read Merger (PEAR) utility to merge reads and DIAMOND (version 0.9.12) aligner algorithm (Buchfink et al. (2015); Zhang et al. (2014)) to align to the RefSeq, SEED Subsystem, and CAZyme databases. See Cantarel et al. (2009); Overbeek et al. (2014).

In Example 11, reads were annotated through a modified version of the Simple Annotation of Metatranscriptomes by Sequence Analysis 2 (SAMSA2) pipeline as described by Wurster et al., Cell Reports, 37, 110113 (2021). See also Westreich et al., BMC Bioinformatics, 19, 175 (2018). Reads are quality controlled and merged with Paired-End Read Merger (PEAR) then aligned against to the CAZyme, Cantarel et al., Nucleic Acids Research, 37, D233-D238 (2009); SEED Subsystem, Overbeek et al., Nucleic Acids Research, 42, D206-D214 (2014); and RefSeq databases, O'Leary et al., Nucleic Acids Research, 44, D733-D745 (2016), with DIAMOND (version 0.9.12), Buchfink, Xie, & Huson, Nature Methods, 12, 59-60 (2015). See Zhang et al., Bioinformatics, 30, 614-620 (2014).

Metatranscriptomic analysis: HUMAnN3. HUMAnN3 (Beghini et al. (2021)) was used to identified changes in gene expression from cleaned metatranscriptomic and metagenomic reads. Reads were aligned to the UniProt/UniRef 2019_01 databases to identify expression of reactions and MetaCyc to identify expression of pathways. MetaPhlAn 3.0 and the ChocoPhlan pangenome database was used to classify reads to bacterial species. Reads aligning to reactions and pathways are normalized to sequencing coverage and reported as copies per million (cpm, Copm) in the metatranscriptomic and metagenomic samples.

Metagenomic and metatranscriptomic analysis with HUMAnN3. In Example 11, metagenomic and metatranscriptomic expression changes were identified using HUMAN3. Beghini et al., Elife, 10, e65088 (2021). Reads were classified to bacterial species using a custom pangenome database. The custom database was built using the MGBC genome database. Youngblut & Ley, PeerJ, 9, e12198 (2021). Gene prediction from assembled contigs was achieved using Prodigal (version 2.6.3), Hyatt et al., BMC Bioinformatics, 11, 119 (2010), with gene duplicates per genome removed using vsearch (version 2.21.1), Rognes et al., PeerJ, 4, e2584 (2016). Genomes were concatenated into a single FASTA file with genes clustered by sequence identity and assigned UniRef90 database, Suzek et al., Bioinformatics, 23, 1282-1288 (2007), annotations with MMseqs (version 13.45111), Hauser, Steinegger, & Söding, Bioinformatics 32:1323-1330 (2016), generating the final annotated FASTA file. A custom protein database was built using the same process but starting with the amino acid output from Prodigal. Classified reads were aligned to this custom protein database to identify functional expression and to the MetaCyc database to identify expression pathways. Caspi et al, Nucleic Acids Research, 38, D473-D479 (2010). Aligned reads are normalized to sequencing coverage and reported as copies per million (CoPM).

Redox potential and pH measurements. Redox potential was measured according to a modified protocol from Husson (2012); Husson et al. (2016) with an Ag/AgCl Reference electrode (Radiometer analytical E21M002, Radiometer Analytical Pt plate electrode 5×5 mm M241 Pt), and a voltmeter. Flash frozen and lyophilized cecal contents were first rehydrated at 1:10 ratio in reverse osmosis water. The rehydrated samples were then vortexed for ten minutes with 10-minute breaks over sixty minutes. The samples were blinded and measured in random order. These samples were then used to measure eH and pH within twenty-four hours or ninety-six hours to account for dissolved gases each sample was vortexed for another minute before measurement. A 0.1M KCL agar plate was the base for the samples. A cut plastic pipette tip was inserted into the agar to hold 300 μL of the cecal extract.

The platinum electrode was inserted into each sample. The reference electrode was inserted into the 0.1M KCL agar. The redox potential was allowed to stabilize for twenty minutes or fifteen minutes before recording the voltage. Electrodes were cleaned between each measurement and placed in the RENOVO cleaning solution from Hach (Loveland, CO, USA) for one minute. The electrode was then rinsed with reverse osmosis-purified water. The electrode was used to measure a 220 mV redox buffer solution to confirm integrity of the electrode before measuring the next sample. The same cecal extracts were used to measure pH using a PH meter and for subsequent validation with ATP assays, 16S rRNA sequencing and quantitative polymerase chain reaction (qPCR) bacterial load.

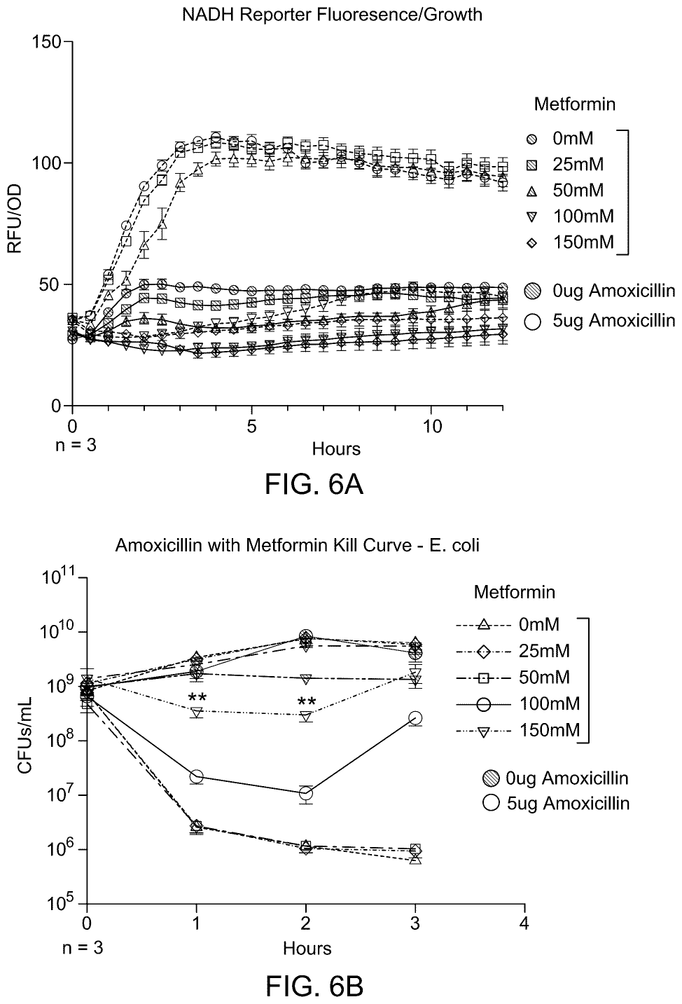

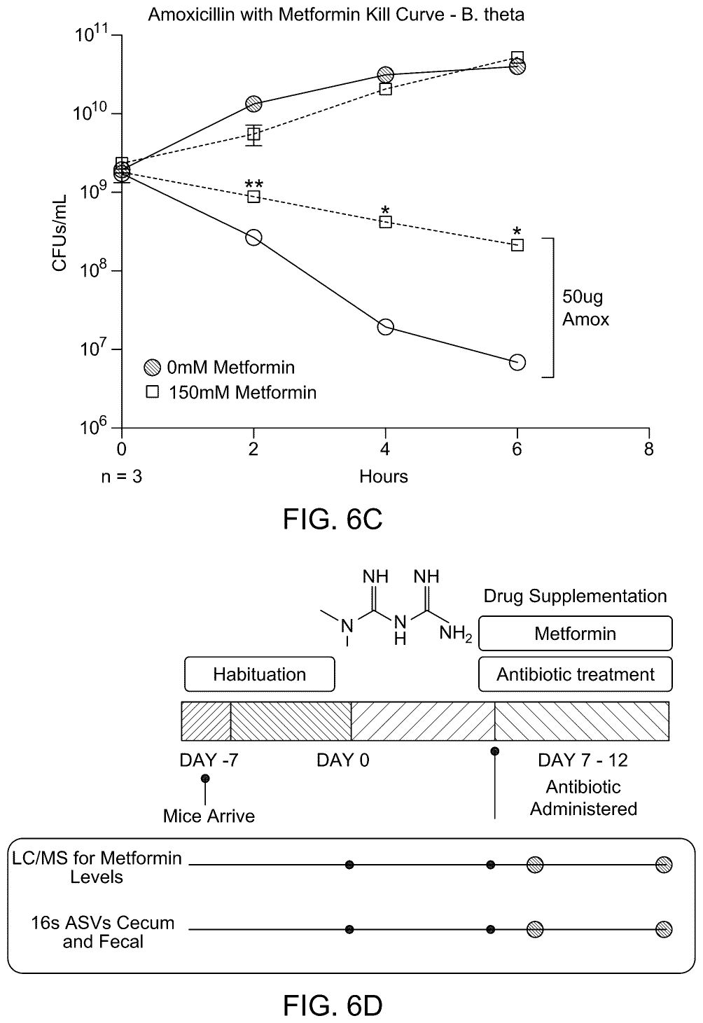

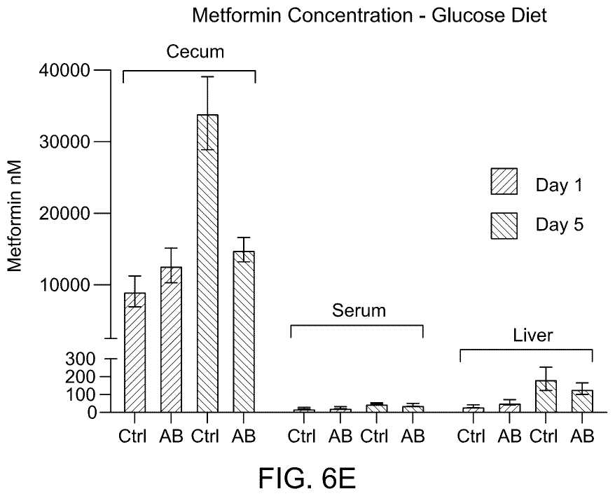

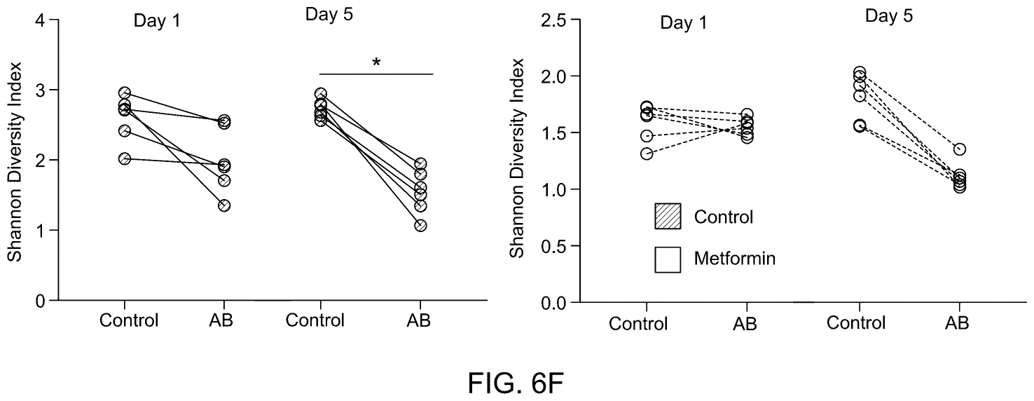

NAD+/NADH reporter: E. coli reporter assay. The NAD+/NADH reporter plasmid (Liu et al., 2019) was transformed into E. coli (MG1655) for growth and fluorescence assays. Overnight cultures of E. coli were diluted 1:1000 in fresh Luria Broth. The diluted cultures were allowed to grow to 0.2 OD before treatment with amoxicillin and metformin. Fluorescence and growth (in OD) were measured in parallel using two plate readers. Data is shown with fluorescence normalized to growth and as separate growth and fluorescence curves. See FIG. 6A-6F.

Amoxicillin kill curves in E. coli and B. theta. Kill Curves for E. coli were done in an aerobic environment and for B. theta in the anaerobic chamber. Overnight cultures s were grown in Luria Broth (E. coli) or mGAM (B. theta). Overnight cultures were diluted 1:100 in fresh media. They were allowed to grow to 0.2 OD before treatment with amoxicillin and metformin. Samples were grown at 37° C. and plated every two hours for six hours in serial dilutions. Colony forming units (CFUs) were measured the following day.

Metformin in vivo supplementation. Metformin hydrochloride (Fisher Scientific, Waltham, MA, USA) was added to powdered mouse diet at 100 mg/kg (w/w) or 0.1% in the glucose diet and homogenized before administration to mice via feeding jars. In the chow diet metformin was added 50 mg/kg (w/w) or 0.05%. See FIG. 6A-6F.

Mass spectrometry for metformin measurement. Plasma, liver and cecal samples were collected for from the sacrificed mice in the two metformin supplementation assays (n=6 mice per group, twenty-four mice/assay). The liver was perfused with phosphate-buffered saline upon collection and the tissue was homogenized with reverse osmosis-purified water at 1 mg/10 μL using a Bead Ruptor at speed 30 for five minutes. Supernatants from the homogenate was collected after centrifugation at 2000×g for five minutes to pellet beads. Cecal contents were also homogenized with reverse osmosis-purified water at 1 mg/10 μL, and supernatants were used for analysis. Metformin extraction and mass spec protocol modified from Chaudhari et al. (2020). For metformin extraction, 30 μL of cecal/tissue homogenates or plasma were added to 70 μL of D6-metformin (ALSACHIM) at 250 μM concentration in methanol. Samples were then vortexed on the Bead Ruptor without beads at speed 10 for ten minutes. Vortexed samples were then centrifuged at 16,000×g for ten minutes and supernatants were used for analysis.

Metformin in biological samples after spiking with D6-metformin (ALSACHIM) and extraction was quantitated with HPLC-MS on an Ultimate 3000, Dionex coupled to Q Executive Classic (Thermo Fisher) with ESI interface. Data acquisition and processing was performed by Excalibur software. The chromatographic separation was achieved on a BEH C18 1.7 μm; 2.1×50 mm (Waters, Milford, MA, USA), at 30° C. Mobile phase consisted of water for phase A and 50/50 methanol/acetonitrile for phase B, both containing 0.2% FA. Separation was optimized using a fast gradient method with mobile phase A/B set to 99%/1% from 0.00 to 0.50 minutes and 99%/1% to 10/90% from 0.5 to 1.5 min and 10/90% from 1.5-1.7 minutes then back after 1.7 minutes for equilibration at 99%/1% from 1.7 to 6.0 minutes with the flow 0.25 ml/min. The mass spectrometer was operated in the positive ion mode in Full MS; at resolution 70,000; AGT target 5 E5 and Scan range 90-300 m/z. Spray voltage and source temperature were set at 3,500 volts, and 320° C., respectively.

ATP assay. Cecal extracts from the eH and pH measurements were used to measure ATP with the BacTiter-Glo Microbial Cell Viability Assay (Promega, Madison, WI, USA) as per manufacturer's instructions. A standard curve was also measured in each assay to confirm assay methods.

qPCR for bacterial load. Quantitative polymerase chain reaction (PCR) for bacterial load determination was done as described by Vaishnava et al. Science, 334(6053), 255-258 (Oct. 14, 2011). Q-PCR analysis of bacterial genomic DNA using iTaq Master Mix (Bio-Rad, Hercules, CA, USA) and universal 16S rRNA gene primers. A standard was constructed referring to cloned bacterial DNA corresponding to a 179 bp section of the 16S rRNA gene amplified using 19s RNA specific primers. Sq values were normalized to the DNA in the sample.

Serum cytokine panel. After animal sacrifice, whole blood was obtained by cardiac puncture and placed in a microcentrifuge tube to coagulate for thirty minutes. The collection tubes were then centrifuged at 13,000×g for ten minutes to separate the serum, which was then transferred to a new microcentrifuge tube and frozen at −80° C. until further processing. When ready, the samples were thawed on ice and divided into a working aliquot and a re-frozen stock aliquot. The working aliquot was analyzed for signs of inflammation in mice using the LEGENDplex Mouse Inflammation Panel (13-plex) (BioLegend, San Diego, CA, USA) flow cytometry kit, following the manufacturer's instructions. The samples were analyzed on the Attune NxT Flow Cytometer (ThermoFisher, Waltham, MA, USA) and then evaluated using the LegendPlex cloud software tool (BioLegend, San Diego, CA, USA).

Statistical analysis. Specific details of the statistical analyses for all assays are outlined in the BRIEF DESCRIPTION OF THE DRAWINGS. All sample numbers represent biological replicates. PERMANOVA was calculated using the adonis method on Bray-Curtis distance matrices calculated from multidimensional scaling of sequencing data using phyloseq. Control samples were compared against antibiotic treated in each group to determine antibiotic effect size. LEfSe (version 1.0) was used to analyze HUMAnN3 outputs on the Galaxy web server using default settings. Metatranscriptomic outputs generated by SAMSA2 were subjected to differential abundance testing using the DESeq2 package (1.24.0) in R (version 3.5.2) under default parameters and included contrast:interaction comparisons. See Love et al. (2014). All DESeq2 results were corrected using the Benjamini-Hochberg method (FDR=p-adj) to account for multiple hypothesis testing. ANOVA, unpaired t tests, and Mann-Whitney U, Kruskal-Wallis tests were performed in Prism GraphPad (version 9.0) without sample size estimation. MaAsLin2 was used to identify significant pathway and reaction annotations from HUMAnN3 outputs. FDR=−log(qval).

In Example 11, initial output analyses were conducted by linear discriminant analysis effect size (LEfSe) Galaxy web server (Galaxy version 1) under default settings. See Segata et al., Genome Biology 12:R60 (2011). Kraken 2/Bracken Metagenomic outputs and SAMSA2 metatranscriptomic outputs were tested for differential abundance using the DESeq2 package (version 1.34.0), Love, Huber, & Anders, Genome Biology, 15, 550 (2014), under default parameters with Benjamini-Hochberg correction. HUMAN3 metatranscriptomic outputs were tested for differential abundance using the microbiome multivariable association with linear models (MaAsLin2) package (version 1.8.0), Mallick et al., bioRxiv (2021), under default parameters. Mann-Whitney unpaired t tests and Kruskal-Wallis one-way analyses of variance (ANOVAs) were performed in GraphPad Prism (version 6.0). All assays represent biological replicate data, and details of specific statistical analyses for all assays are defined in figure legends.

Data availability. The short-read metagenomic and metatranscriptomic sequencing data generated in this study have been deposited in the NCBI SRA database. The metagenome-assembled genomes (MAGs) have been deposited to NCBI GenBank. The 16s sequencing data has been depositing to NCBI SRA. BioProject accession code for all sequences associated with this study is PRJNA984334. The DESeq2, LDA, and MaAsLin2 results generated in this study are provided in the Supplementary Information. Source Data contains all PERMANOVA stats information. Databases used in this study (MMTV, accession NC_001503), (MOV, accession NC_001506.1), SILVA 128 LSU and SSU Parc ribosomal RNA databases, RefSeq, SEED Subsystem, CAZyme databases, UniProt/UniRef 2019_01 databases, and SILVA 132 99% database.

The following EXAMPLES are provided to illustrate the invention and should not be considered to limit its scope in any way.

A high-fiber diet can prevent antibiotics from increasing aerobic respiratory metabolism, reducing antibiotic-induced gut dysbiosis. These EXAMPLES disentangle the effects of fiber supplementation on the biochemistry of the gut microbiome and how it can modulate community composition as an effective therapeutic. The increase in respiratory metabolism seen during-antibiotic treatment can also be modulated with pharmaceuticals such as metformin and other modulators of bacterial biochemistry. Many plant-derived fibers are being explored as microbiome therapeutics. These EXAMPLES show that other methods of directly changing bacterial metabolism with drugs could be an avenue for therapeutics.

### Example 1

Fiber Protects from AID Before, During and after Antibiotic Treatment.

This Example shows that fiber-supplementation has a beneficial impact on the gut microbiome of antibiotic-treated mice.

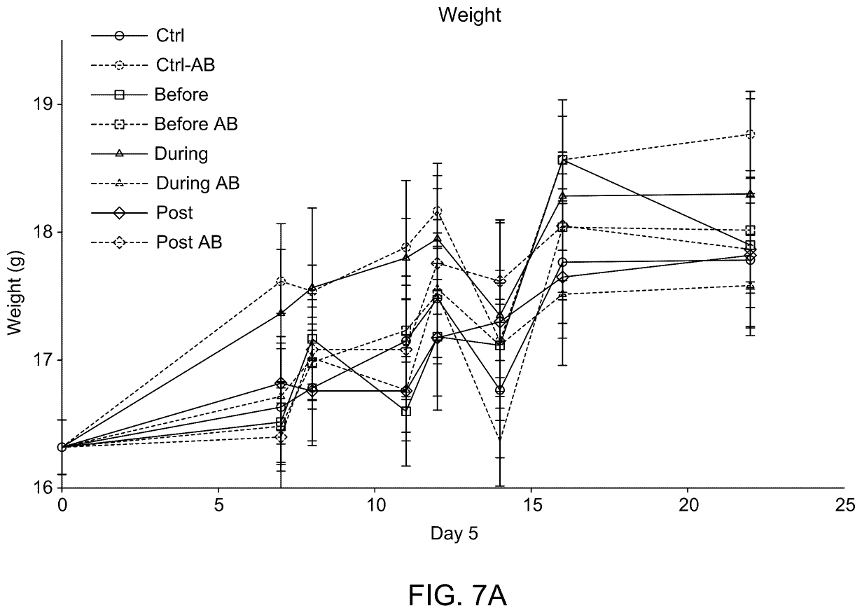

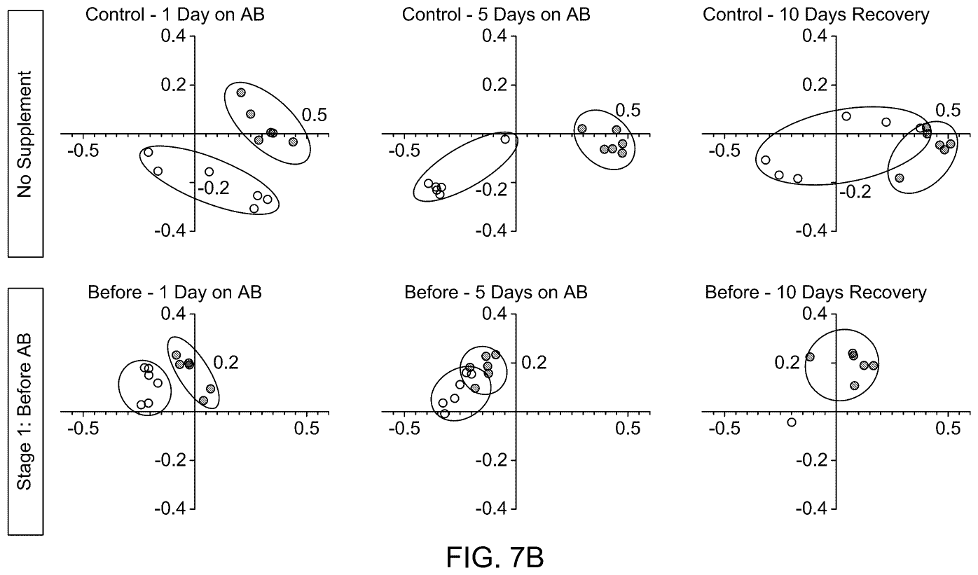

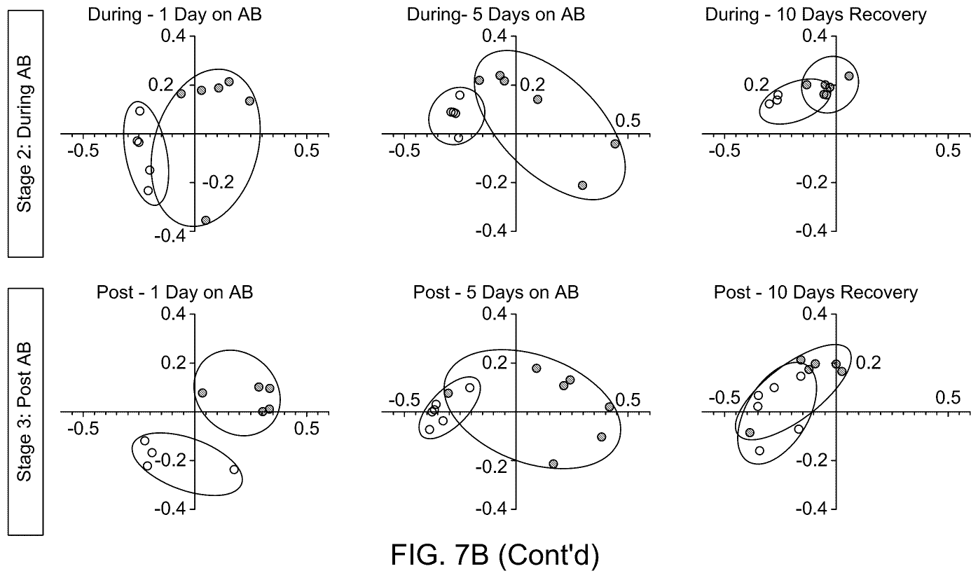

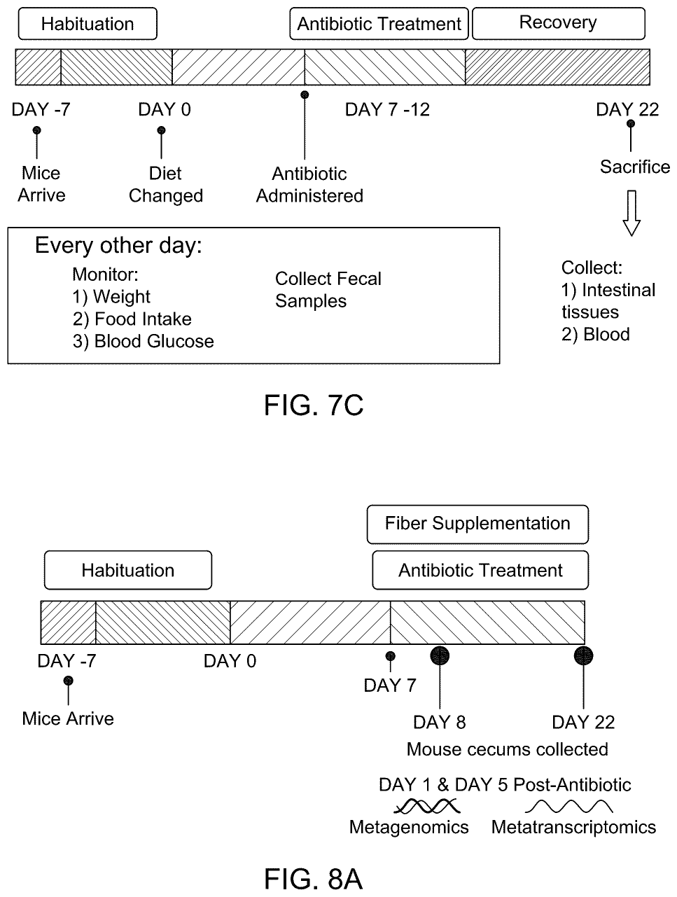

The inventors performed longitudinal 16S rRNA sequencing of mouse fecal samples to assess the optimal stage of fiber supplementation on post-antibiotic recovery. The results showed that dietary carbon source modulates antibiotic-induced changes in the gut microbiome with glucose exacerbating the dysbiotic effects of antibiotics. No significant difference in weight were seen between groups. See FIG. 7A. Glucose supplementation alone was seen decreasing microbial diversity through-out the assay.

During antibiotic-treatment, the S1: before group had a significantly lower (p<0.05) initial reduction in diversity and a more complete recovery as compared to the glucose group. Fiber supplementation during antibiotic treatment conferred significant protection in both the treatment (p<0.01) and recovery stage (p<0.001). Fiber supplementation after antibiotic-treatment (S3: after) led to improved recovery with an increase in microbial diversity compared to the glucose group. Supplementation with fiber at various stages led to significant changes in microbial composition during treatment. The inventors found that glucose exacerbates antibiotic-induced dysbiosis by increasing inflammatory phyla such as Proteobacteria. See FIG. 7A-7C. Bacterial phyla responsible for carbohydrate degradation such as Bacteroidetes and Firmicutes are higher in mice on fiber-supplemented diets. See FIG. 7A-7C. These observations were for all tested stages of fiber-supplementation.

The inventors also observed that supplementing single-purified fibers at 5% composition was also beneficial to microbiome recovery post-antibiotic treatment. See FIG. 7C. A modification to diet should be able to affect microbiome recovery post-antibiotic treatment. For a translational medicine advantage, supplementation at the time of antibiotic administration is as effective as before treatment.

### Example 2

### Fiber Reduces AID and Glucose Exacerbates.

The results of this Example show that diet affects microbiome diversity metrics during antibiotic treatment.

The 16S rRNA sequencing provides some insight into community ecology. The low resolution of data makes it difficult to assess community composition and structure. See Bharti & Grimm, 2021).

The inventors used shotgun metagenomic sequencing of mouse cecal contents Day 1 and Day 5 post-antibiotic treatment (n=6). Mice had no significant changes in intestinal histopathology Day 1 and Day 5 post-antibiotic treatment between the tested diets.

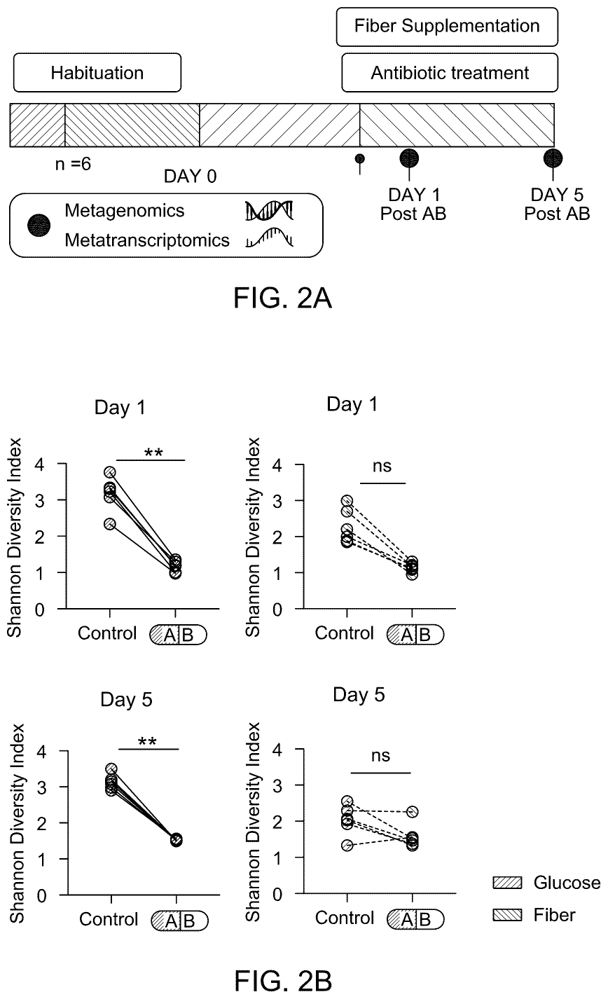

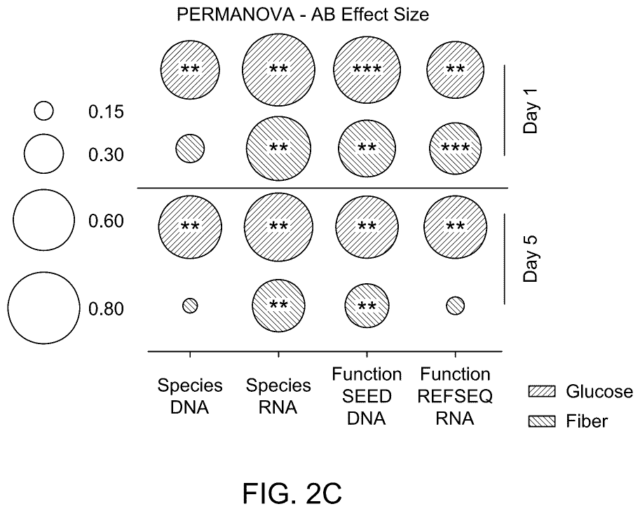

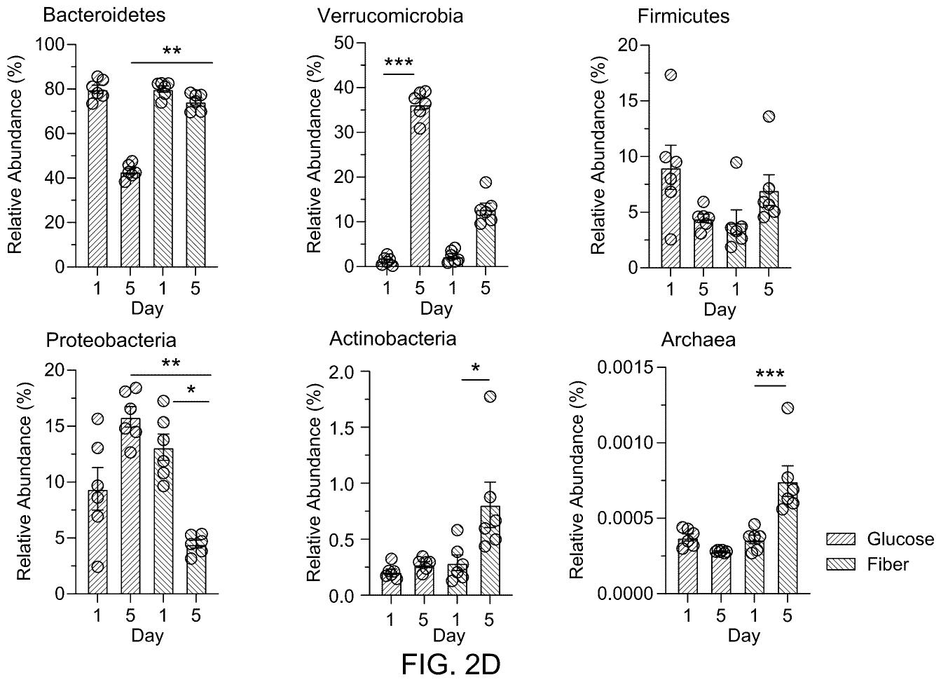

The inventors analyzed using 16S sequencing. They found that mice on the glucose diet had a significantly higher decrease in alpha diversity after antibiotic administration at both timepoints. See FIG. 2A. Using centroids determined from the Bray-Curtis distance of beta-diversity, the inventors found that the antibiotics led to a larger shift on glucose supplemented mice compared to fiber Day 1 and Day 5 post-antibiotic treatment. See FIG. 2B.

The inventors used de novo gene assembly of the metagenomic data to identify fifty-four high-quality metagenome-assembled genomes (MAGs) across the samples. As compared to short-read metagenomic data, de novo gene assembly can reduce the effects of sequencing error, genomic repeats and uneven sequencing coverage on interpreting metagenomic data. See Bharti & Grimm (2021). Using linear discriminant analysis of the relative abundance of metagenome assembled genomes in samples, the inventors identified significant changes in microbiome composition associated with glucose and fiber supplementation during antibiotic treatment. Glucose supplementation was associated with an increase of MAGs in the Bacteroidetes, Proteobacteria, and Verrucomicrobia phyla five days post-antibiotic treatment. See FIG. 2D. Relative abundance of total MAGs within each phyla are shown in FIGS. 2E, 2F, 2G, and 2H.

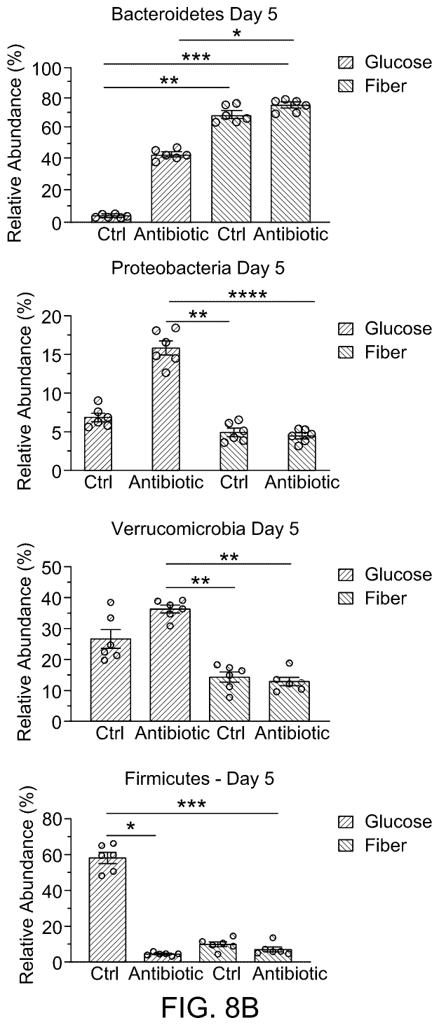

The inventors observed larger shifts in microbiome composition in glucose-supplemented mice compared to fiber supplementation. See FIG. 2H. This observation shows that antibiotic disruption is exacerbated by glucose supplementation and remediated by fiber. The inventors observed similar patterns using analysis of short read unassembled metagenomic data. See FIG. 8B.

### Example 3

### Diet has Divergent Metabolic Response on the Gut Microbiome: Glucose Increases Oxidative Metabolism and Fiber Represses.

This Example shows that the microbiome of fiber supplemented mice increased fermentation of polysaccharides. This Example shows that fiber supplementation can encourage protective fermentative metabolism by reducing gut redox potential and oxygen and leading to protection from the damaging respiratory burst seen post-antibiotic treatment.

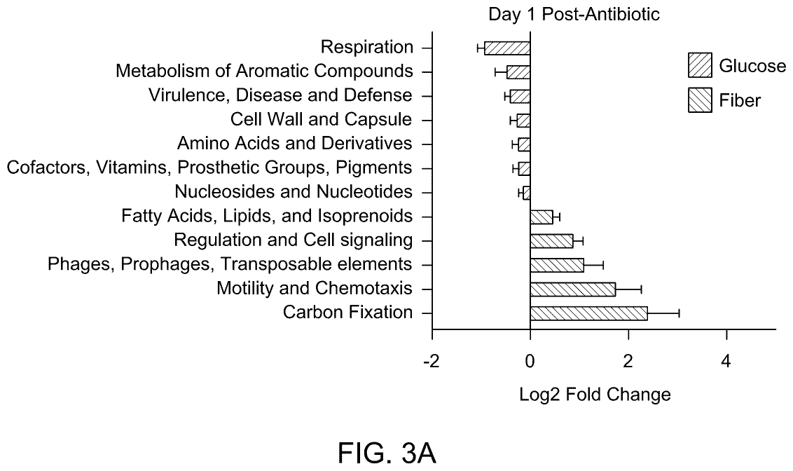

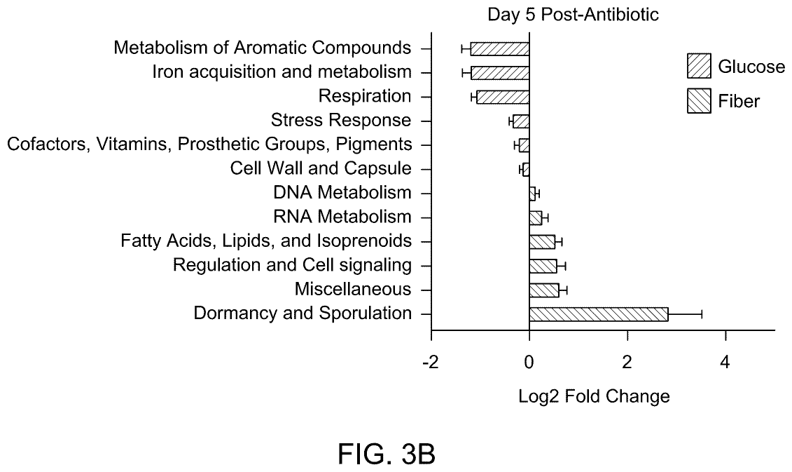

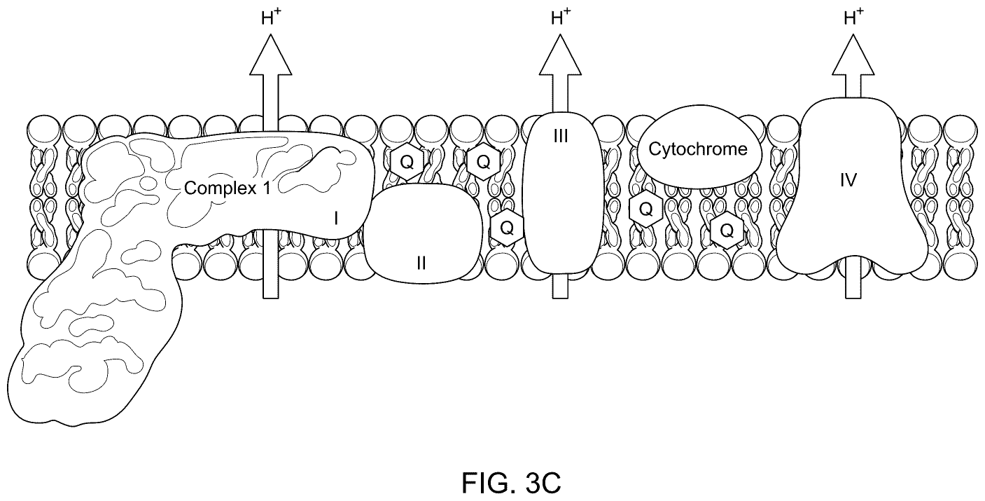

To determine changes in metabolic function and how they affect antibiotic susceptibility in the gut microbiome, the inventors used metatranscriptomic sequencing of the mouse cecal samples to observe metabolic signatures in the gut microbiomes of mice fed glucose and fiber. Using the SEED subsystem database, the inventors found significant increases in pathways involved in respiratory metabolism in glucose supplemented mice during antibiotic treatment (p<0.05). See FIGS. 3A and 3B). Fiber supplementation was associated with increased metabolic pathways associated with dormancy, carbon-fixation and fatty-acid metabolism. This result shows that glucose and fiber have divergent effects on the bioenergetics of gut bacteria, and this may contribute to the differences seen in taxonomic response.

The inventors looked at gene expression of proteins involved in the electron transport chain. Using differential abundance analysis of transcriptomic reads aligned to the RefSeq database, they found that glucose was significantly associated with increased electron transport chain activity during antibiotic challenge. Expression of complex 1, flavoproteins and cytochromes was greater in glucose-supplemented mice post antibiotic treatment (p<0.0001) FIG. 3D, 3E. These iron-sulfur cluster proteins are for energy-converting electron transfer reactions that bacteria use for energy. See Khademian & Imlay (2021). Their overall increase in the glucose diet shows this community has more active respiratory metabolism. The fiber diet has significantly less expression of these proteins, suggesting this community has less metabolic activity.

These data show the divergent effects of glucose and fiber on gut microbiome bioenergetics, with glucose increasing aerobic respiratory metabolism and fiber leading to slower metabolic processes such as carbon-fixation and fermentation. Fiber supplementation increases fermentative metabolism and reduces gut redox potential.

To better elucidate the mechanism behind this metabolic shift in response to the tested diets, the inventors used HUMANN3 biobakery3 to identify changes in biochemical processes across the bioenergetic scale. Stratification of bacterial activity by redox processes can improve the understanding. See Miller et al. (2021). Further organizing these redox processes from low to high redox potential can provide an estimate of the thermodynamically favorable energetic shifts in a given microbial community. See Husson (2012); Million & Raoult (2018).

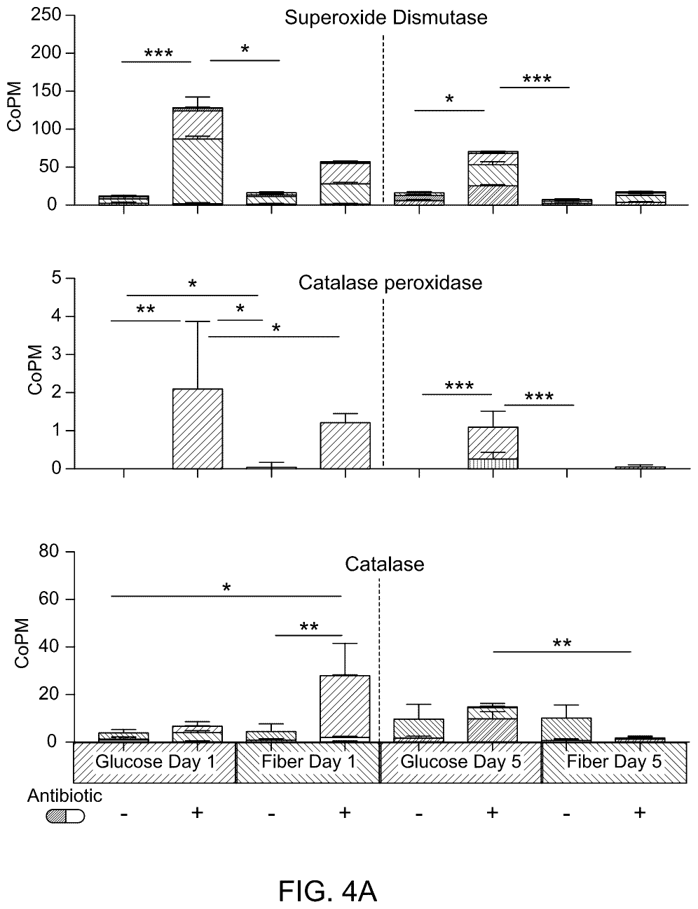

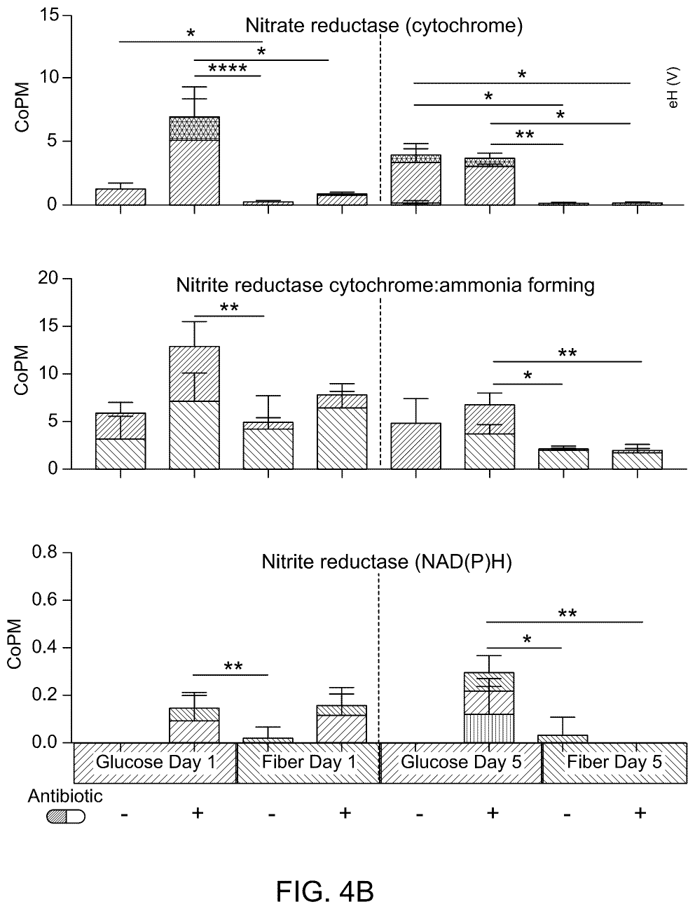

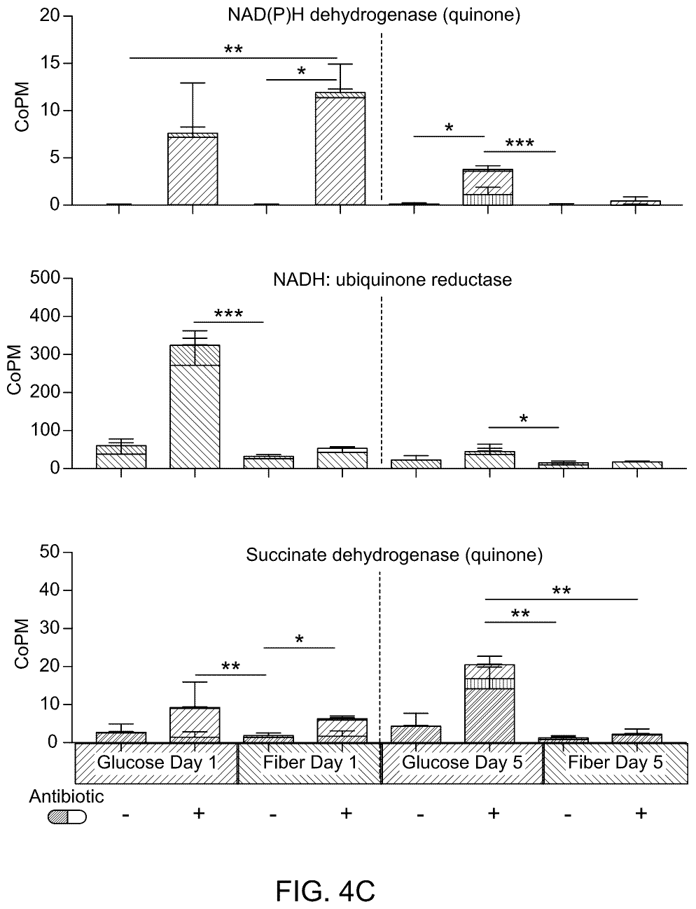

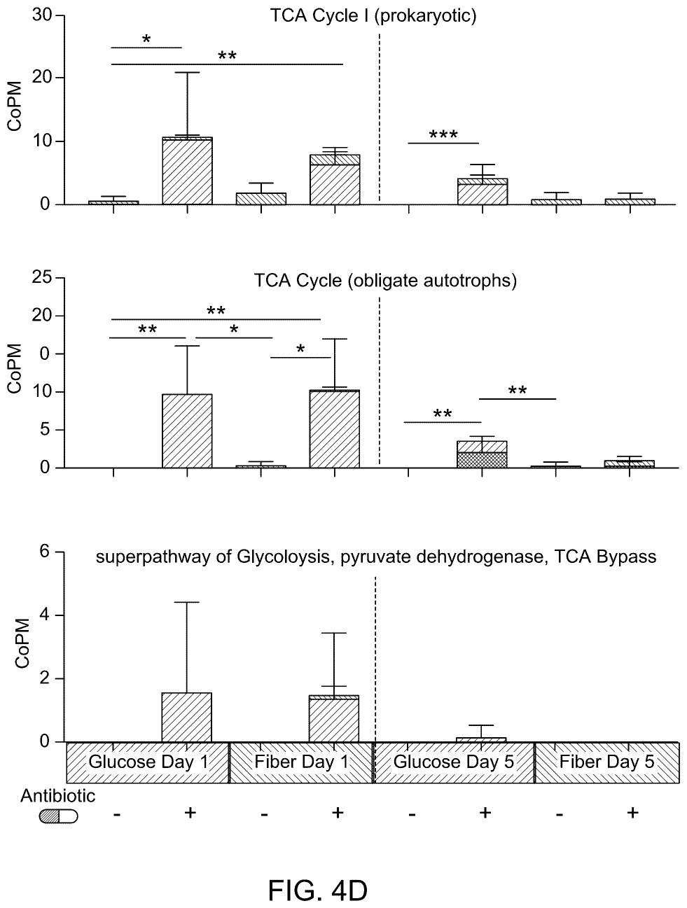

The inventors searched the metatranscriptomic dataset for biochemical reactions based on electron acceptors and redox potential. They observed significantly increased metabolic activity of pathways involving oxygen and nitrate as terminal electron acceptors post-antibiotic treatment in the glucose diet compared to the fiber diet. See FIG. 4A-4B. They also saw increased respiration and ETC activity indicating more oxidative metabolism. See FIG. 4C-4D. Bacteria contributing to these transcriptional changes were predominantly from the Proteobacteria phyla although Bacteroides thetaiotaomicron could have a large function in this increase in respiratory activity post-antibiotic treatment. FIG. 4A-4D. The biochemical reactions involved in these pathways have high redox potentials indicating that the chemical environment of the gut on the glucose diet is more energetic. Baltsavias et al. (2020).

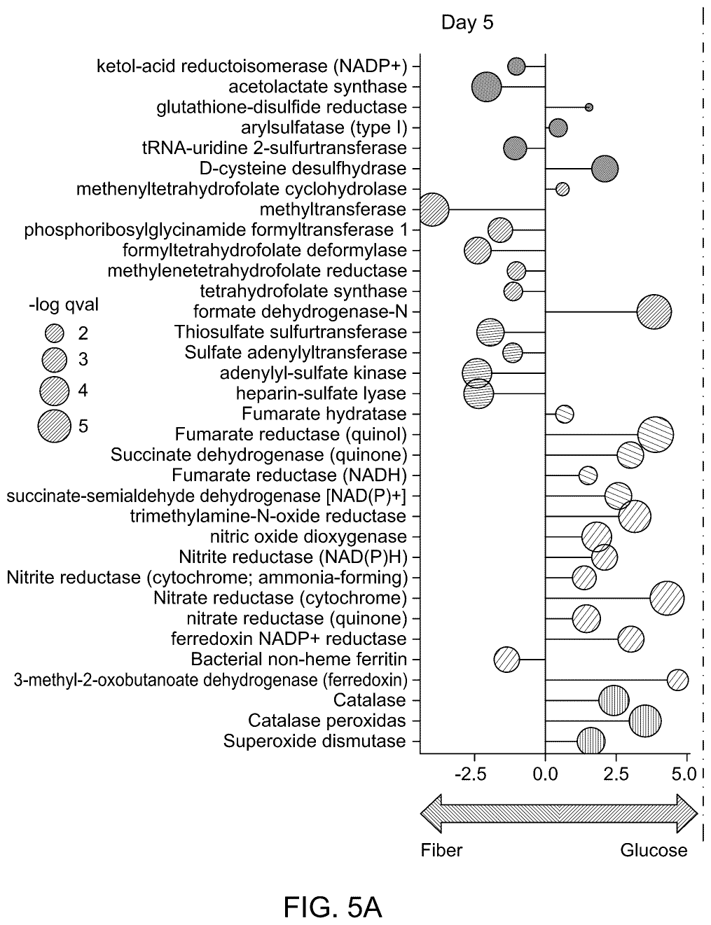

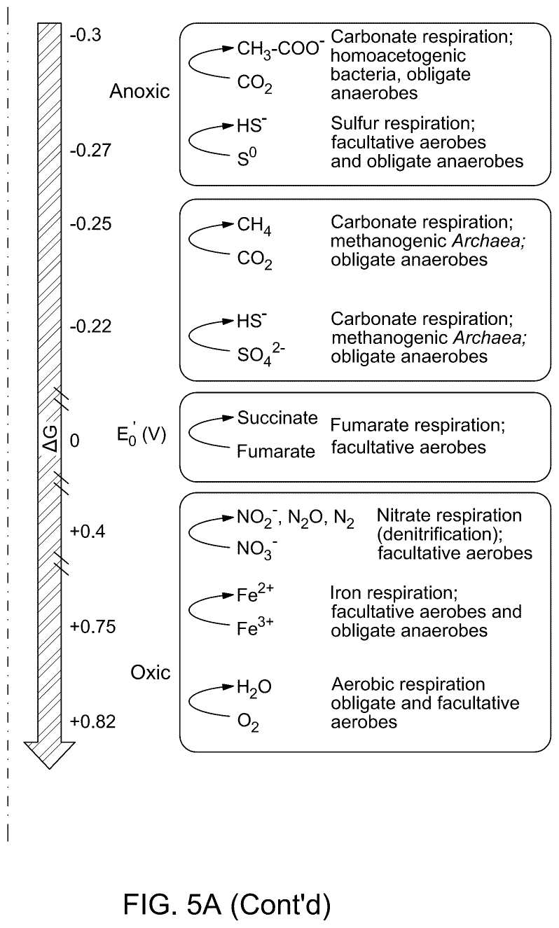

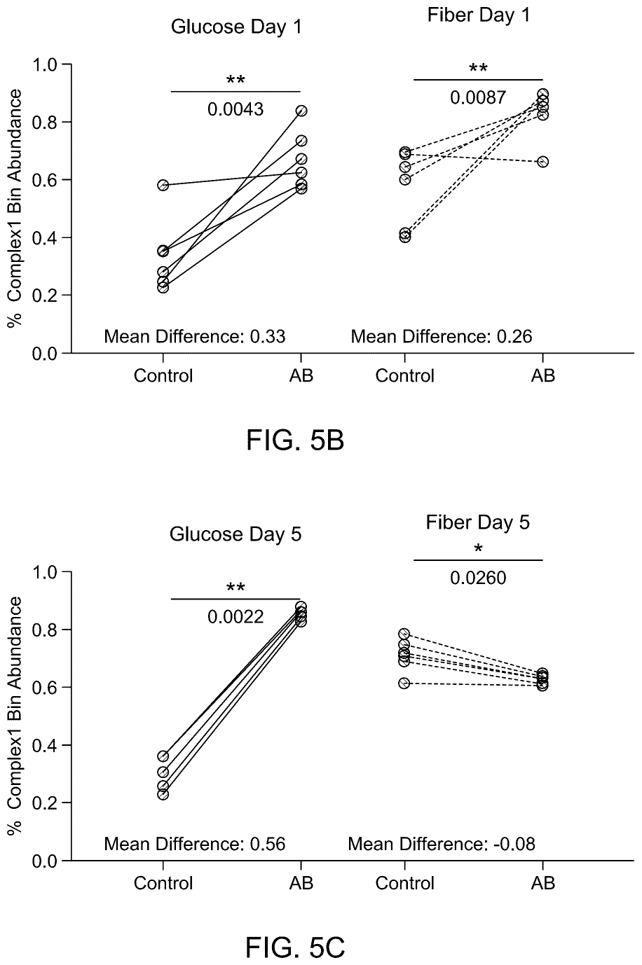

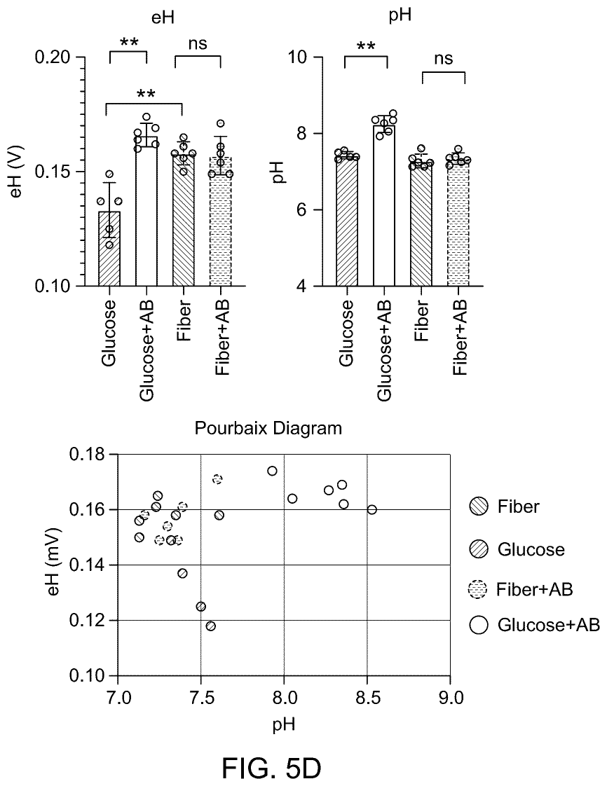

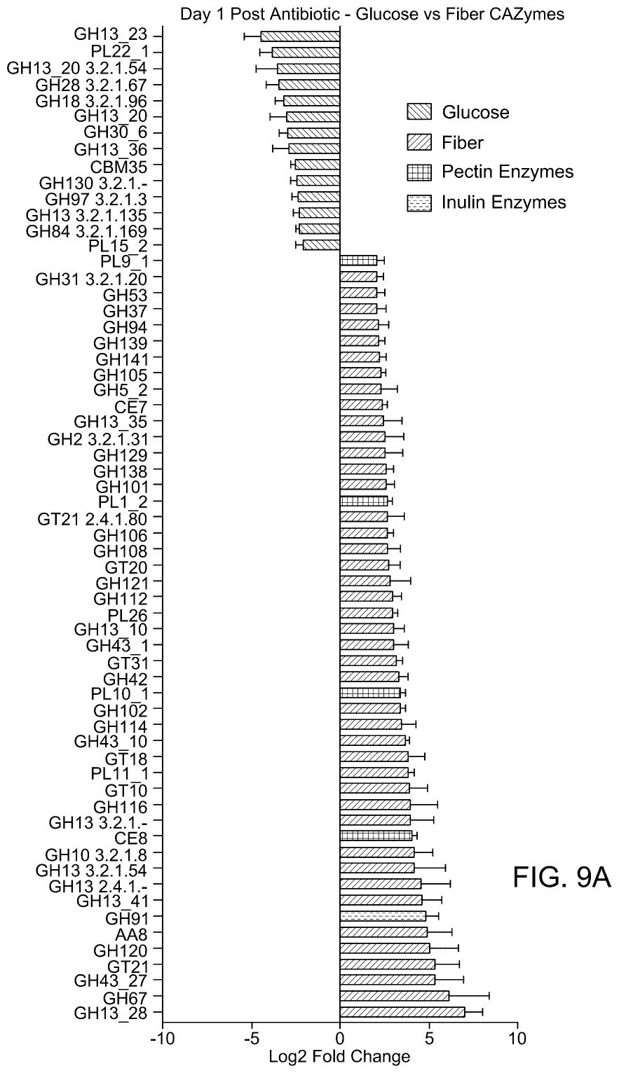

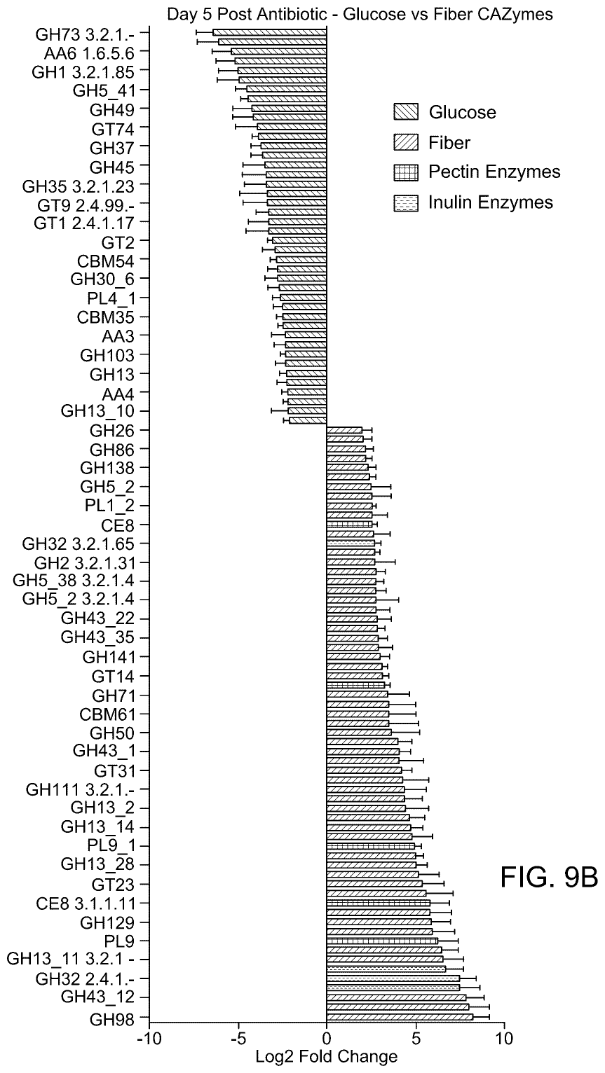

To determine if these changes were contributing to a larger bioenergetic shift in the community, the inventors looked at the expression of several important biochemical reactions and ordered them to by terminal electron acceptors and redox potential. Antibiotics increased relative expression of high redox reactions in the glucose and fiber diets. This increase was much more significant in the glucose diet. See FIG. 5A-5D. This increase in gut redox potential thermodynamically selects for increased respiratory activity and restricts biochemical activity of bacteria that rely on fermentative metabolism. Gut commensals associated with improved health use fermentation to create short-chain fatty acids and maintain an anaerobic environment in the gut. See Carlson et al. (2017). Fiber supplementation was associated with increased expression of carbohydrate-active enzymes (CAZymes) that were involved in polysaccharide degradation. The fiber cocktail was associated with increased expression of total CAZymes and fiber specific CAZymes involved in the degradation of pectin and inulin. See FIG. 9A-9B.

This result shows that the microbiome of fiber supplemented mice increased fermentation of polysaccharides.

Linear discriminant analysis of this dataset identified several pathways significantly associated with the fiber diet. These include carbon fixation pathways such as the Calvin Benson-Bassham cycle. These also include pathways involved in increased production of Coenzyme A which could be another indicator of increased carbon fixation. See Metabolic pathways to an anaerobic environment were significantly associated with the fiber-group suggesting this diet reduces oxygen in the gut and protects from respiratory metabolism. This increase in carbon fixation could be due to increased CO₂ over O₂ as suggested by Steffens et al. (2021). These data contrast with the aerobic oxidative pathways induced in the glucose diet. Catabolic oxidative pathways such as glycolysis, the tricarboxylic acid (TCA) cycle, and the pentose phosphate pathway were associated with the glucose diet by Day 5 of the assay. The glucose diet is more associated with catabolic oxidative metabolism while the fiber diet was associated with anabolic reductive metabolism.

This analysis shows that fiber supplementation can encourage protective fermentative metabolism by reducing gut redox potential and oxygen and leading to protection from the damaging respiratory burst seen post-antibiotic treatment.

### Example 4

### Glucose Increases Abundance of Complex 1 Bacteria Post-Antibiotic Treatment.

The transcriptomic data suggest that ETC activity, specifically complex 1, is involved in the metabolic shift seen in the data. Recent phylogenomic measurements found that ˜50% of bacteria have complex 1. See Spero et al. (2015). These bacteria are mostly in the Proteobacteria phylum.

Complex 1 can be a sign of the bioenergetic capacity. Based on the observed statistically significant increase in genes for subunits of complex 1 (FIGS. 3A-3C), and the larger shift in redox potential (FIGS. 4A-4D and FIGS. 5A-5D), glucose may drive the community composition for increased abundance of complex 1 bacteria. The shift seen in higher redox potential activity may restrict the metabolic activity and survival of fermentative anaerobes. An increase in redox potential thermodynamically restricts the metabolism of bacteria with lower bioenergetic capacity, favoring bacteria capable of high-energy electron transfers.

To understand how the metabolic shift seen in the respective diets was contributing to the community composition, the inventors used Phylophlan3.0 to search the metagenome assembled genomes for presence of complex 1. They found six metagenome assembled genomes in the dataset that contain complex 1 and observed significant changes in their relative abundance.

Compared to the great amount of complex 1 in all bacterial genomes, the anaerobic environment of the gut explains the lower abundance in the samples (6/54). Both diets led to a spike in complex 1 bacteria 1-day post-antibiotic treatment. See FIGS. 5B and 5D. By Day 5 of the assay the glucose diet continued to have greater abundance of complex 1 bacteria compared to the control. The fiber diet had a decrease. See FIGS. 5C and 5E. This increase was limited to four of the six MAGs identified as containing complex 1. MAGs classified to Muribaculaceae and Bacteroidales did not show an antibiotic-induced increase in abundance. Although presence of complex 1 can improve survival in a high redox environment, many other factors can function in the fitness of a bacterium such as resistance genes, growth rate and competition for carbon sources. Using HUMAnN3, the inventors identified changes in abundance of beta-lactamase expression and identified significant antibiotic-induced increases in Bacteroides thetaiotaomicron and A. mucinophila. See Korry et al. (2020). This expression of beta-lactamase improves the survival of bacteria in response to antibiotic treatment and could explain the large abundance of these bacteria seen by Day 5 of the assay. The environment of the gut also creates chemical gradients with a specific spatial organization of bacteria that drive composition. See Earle et al. (2015); Miller et al. (2021). Because of this heterogeneity, the redox environment may not have equal effects on all bacteria found in the gut.

The inventors found that that glucose supplementation can increase gut redox driving the microbiome composition to have more complex 1 bacteria compared to a fiber-supplemented diet.

Fiber supplementation protects from antibiotic-induced increase in gut redox. To assess if the changes seen in the metagenomic and metatranscriptomic data translate to physiological changes in the gut, the inventors used published methods to measure the chemical redox potential in the cecal contents of the mice. See Husson (2012); Husson et al. (2016); Reese et al. (2018).

The inventors first confirmed these methods on mice given a standard chow diet and found that antibiotics increased the chemical redox. These results corroborate with other measurements of the effect of antibiotics on redox potential. Reese et al. (2018).

The inventors then measured the chemical redox of cecal contents from mice given the glucose and fiber diets five days post-antibiotic treatment. They chose this time point based on the sequencing data which suggested large changes in complex 1 usage by Day 5 of the assay. Only the glucose diet post antibiotic treatment was associated with a significant increase in gut redox. Redox potential (eH) is also affected by the pH of the environment. See Husson (2012).

The inventors measured these in parallel and mapped the data on a Pourbaix diagram. These data show that diet changes the chemical environment of the gut contributing to changes in the biochemical activity of gut microbes. Antibiotic-induced changes in the chemical environment of the gut were more significant on the glucose diet compared to the fiber diet indicating the protective capacity of fiber to buffer gut redox.

### Example 5

Metformin Protects Bacteria from Antibiotics In Vitro.

The data in the EXAMPLES above show that a fiber-supplemented diet can reduce gut redox potential. A fiber-supplemented diet can select for bacteria with less respiratory activity. ETC proteins like complex 1, may have a large function in the metabolic shift observed post-antibiotics and associated changes in microbiome composition. Disruption of ETC proteins should have a similar protective effect against antibiotics in gut bacteria.

The inventors used metformin, the pharmaceutical inhibitor of complex 1, combined with antibiotic treatment in vitro and in vivo. Metformin is widely used as the first line treatment for diabetes. Metformin has been investigated as a mitochondrial complex 1 inhibitor. See Li et al. (2020); Silamikele et al. (2021).

There continues to be debate on the mechanism of action of metformin. The previous hypotheses of reducing gluconeogenesis in the liver were questioned. See Rena et al. (2017). The chemical understanding of metformin as a metal chelator is because of the five nitrogen atoms in its structure, which create a strong a positive charge attracting metal electrons. Stynen et al. (2018). Some have suggested that metformin acts as an iron-chelator in cells. They suggested that iron chelation could be the driving factor in its ability to reduce complex 1 activity and respiratory metabolism in mitochondria. Stynen et al. (2018). Metformin may protect E. coli from the antibiotic bleomycin via metabolic inhibition and ROS reduction. See Li et al. (2020).

The inventors used a transcriptionally based redox biosensor described by Liu et al. (2019) to test the effects of metformin on the activity of the bacterial NADH/NAD+ ratio in vitro as shown by an increase in fluorescence. This ratio is a stand in for cellular bioenergetics with a high ratio indicative of increased catabolism

The inventors then treated these bacteria with metformin and measured their growth and fluorescence. Antibiotic treatment significantly increased fluorescence while metformin suppressed this increase. These data corroborate with published data about antibiotic-induced increases in redox potential. Dwyer et al. (2014). This result showed that metformin can decrease NADH/NAD+ ratio as determined by this biosensor. The decrease in the NADH/NAD+ ratio shows that metformin can inhibit metabolic activity. This effect could be related to disruption of iron-sulfur clusters. Stynen et al. (2018).

The inventors then tested the survival of bacteria treated with metformin against amoxicillin. Increasing concentrations of metformin improved the survival of bacteria challenged with amoxicillin. See FIG. 6B. These assays were conducted aerobically in E. coli.

The inventors then tested the effects of metformin on antibiotic susceptibility in the anaerobic gut commensal Bacteroides thetaiotaomicron to test for the same protection in an anaerobic environment. Metformin was effective anaerobically as well and showed significant protection from amoxicillin in B. theta. See FIG. 6C.

These data show that the predicted activity of metformin on mitochondrial complex 1 can be extended to bacterial electron transport proteins. Using metformin should be able to be used for metabolic control of gut bacteria that can be implemented to protect bacteria from antibiotic challenge.

### Example 6

### Metformin Treatment In Vivo Impacts Antibiotic-Induced Dysbiosis in the Mammalian Gut

This Example shows that the effects of antibiotics on the gut microbiome are dampened by metformin administration.

The inventors used metformin supplemented diets and supplemented the control low-fiber diet with 100 mg/kg of metformin hydrochloride (MPI Chemicals) during antibiotic treatment for five days. FIG. 6D. The inventors collected serum, livers and cecums from these mice to compare the distribution of metformin in the host using LC/MS. Chaudhari et al. (2020). Metformin levels were significantly higher in the cecum compared to the liver and serum. See FIG. 6E. This result was expected because metformin is a cationic hydrophilic drug shown to have low host absorption. See Chaudhari et al. (2020); Zake et al. (2021). This high differential in host versus gastrointestinal tract availability makes it a good candidate for targeting the gut microbiome (metformin-gut).

Metformin should be able to act on bacteria with redox dependent metabolism by either inhibiting their growth or affecting their susceptibility to antibiotics. Metformin should be able to eliminate the antibiotic-induced bloom in Proteobacteria while protecting native fermentative microbes.

The inventors then extracted DNA from the cecal contents of mice Day 1 and Day 5 following antibiotic treatment and used 16S rRNA sequencing to investigate changes in gut microbiome composition. Metformin alone led to changes in the microbiome composition and significant changes in bacterial diversity by Day 5 of the treatment. FIG. 6F. Metformin changed the baseline microbiome of mice that did not receive antibiotics. Shannon diversity indices of these microbiomes suggest that the effect of antibiotics on microbial diversity is reduced with metformin supplementation. FIG. 6F. Mice given the metformin diet displayed a smaller decrease in diversity because of antibiotic treatment compared to untreated controls.

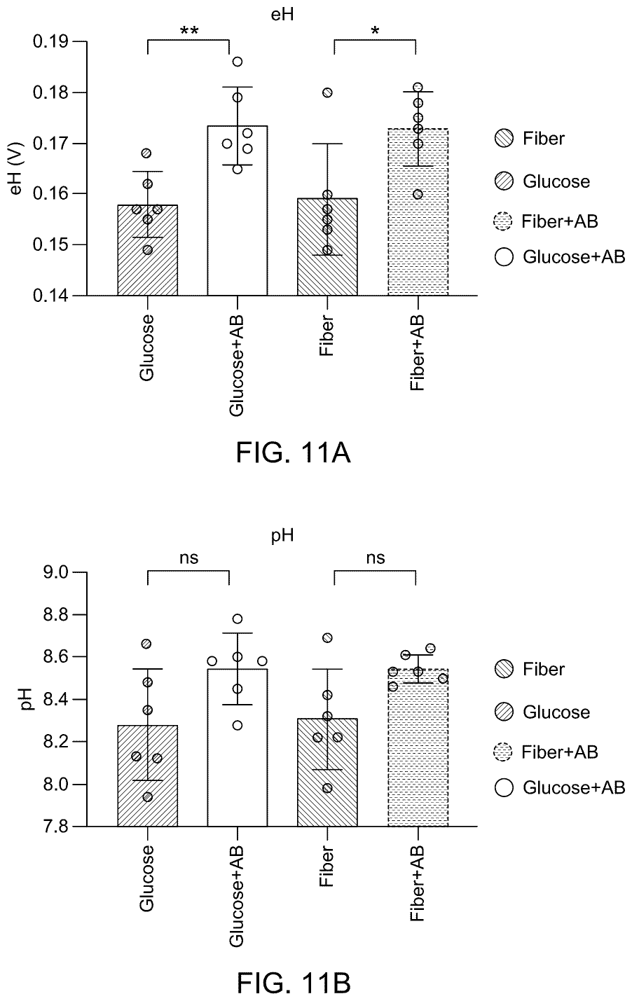

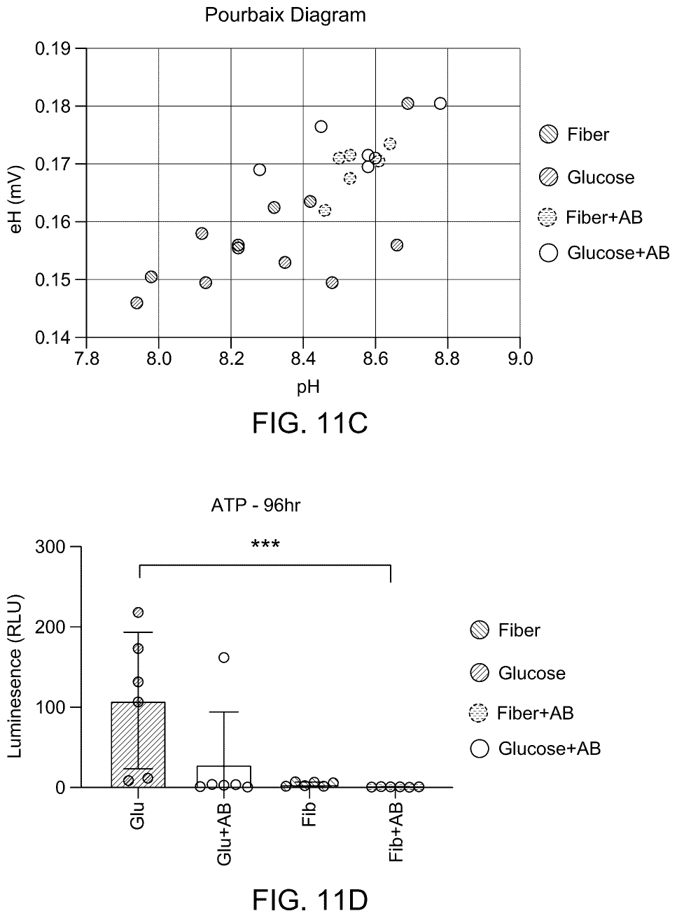

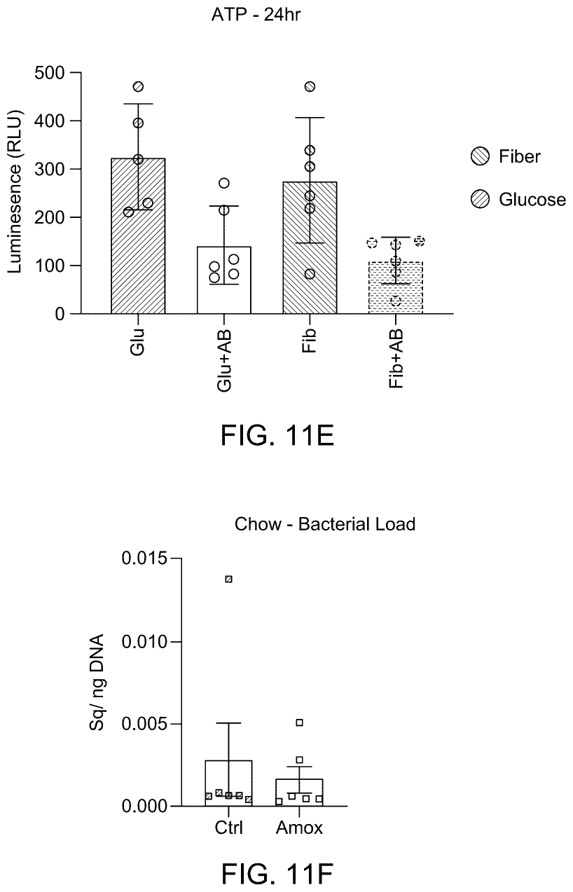

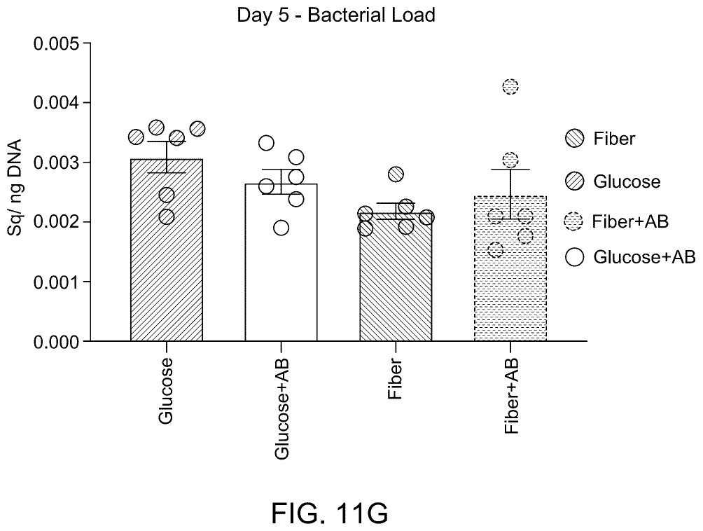

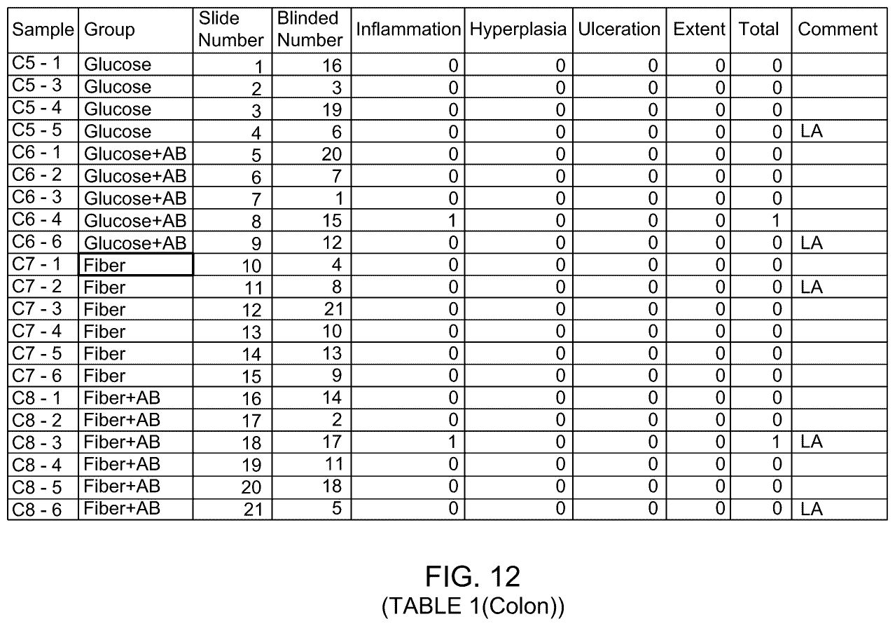

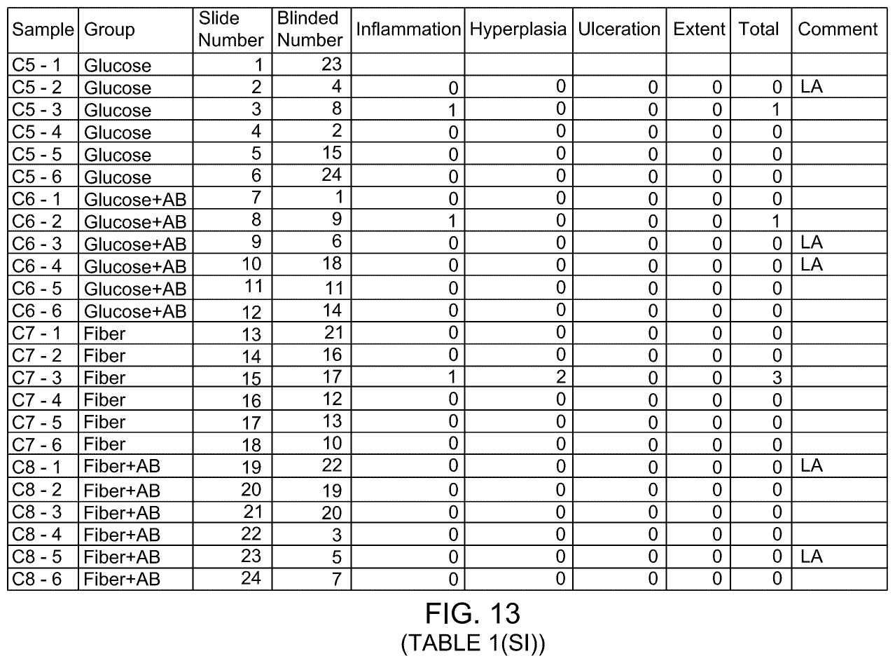

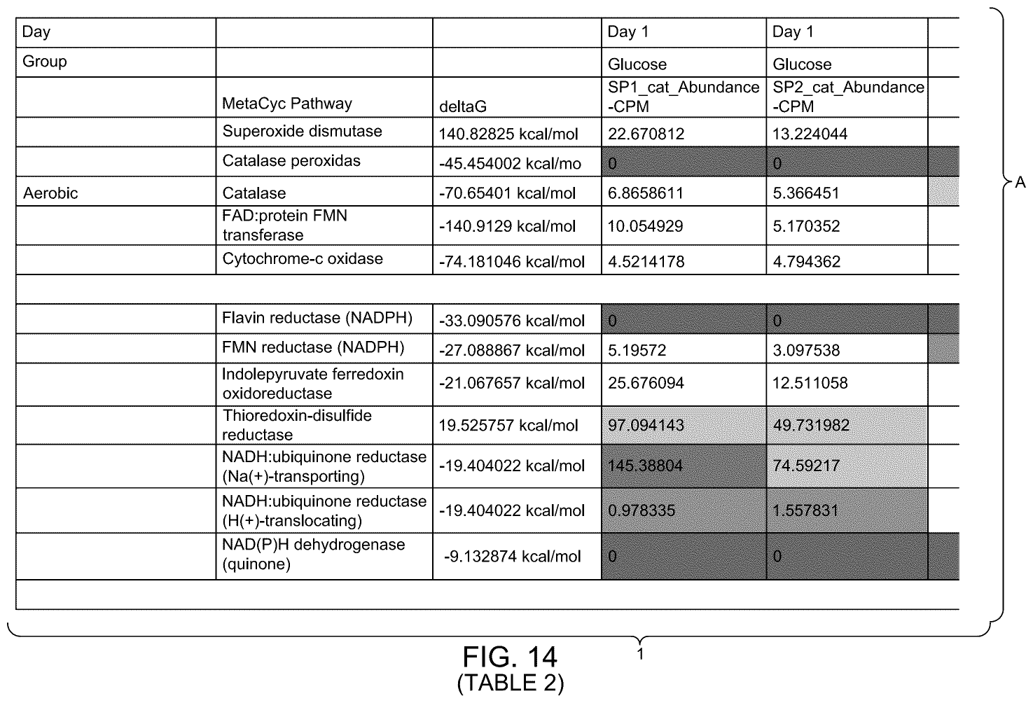

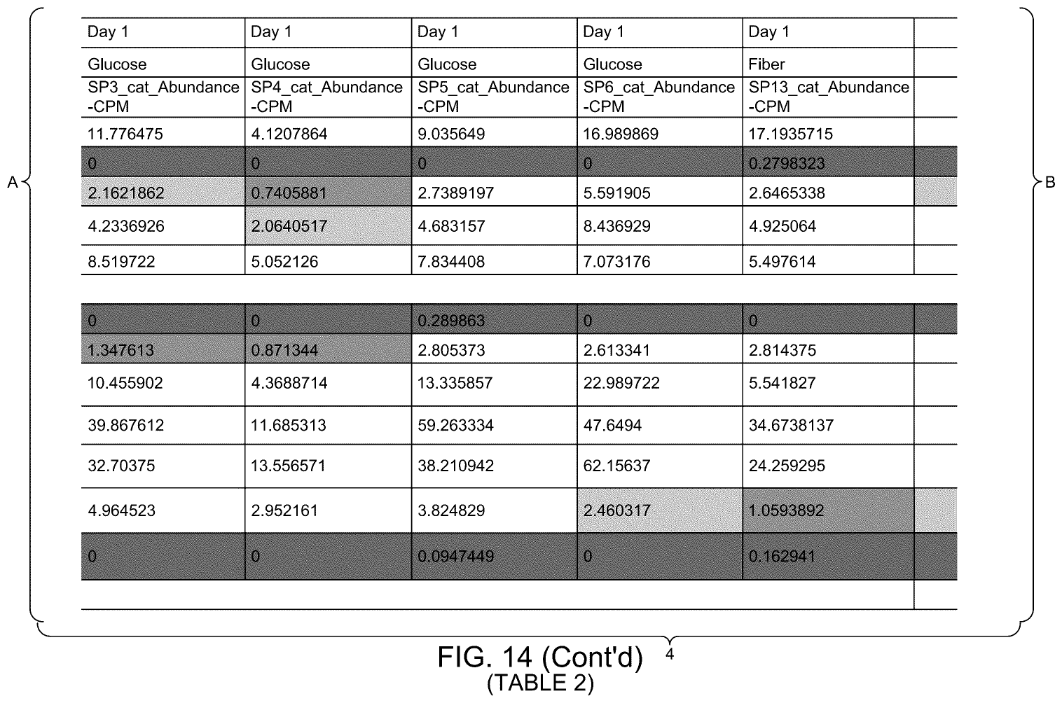

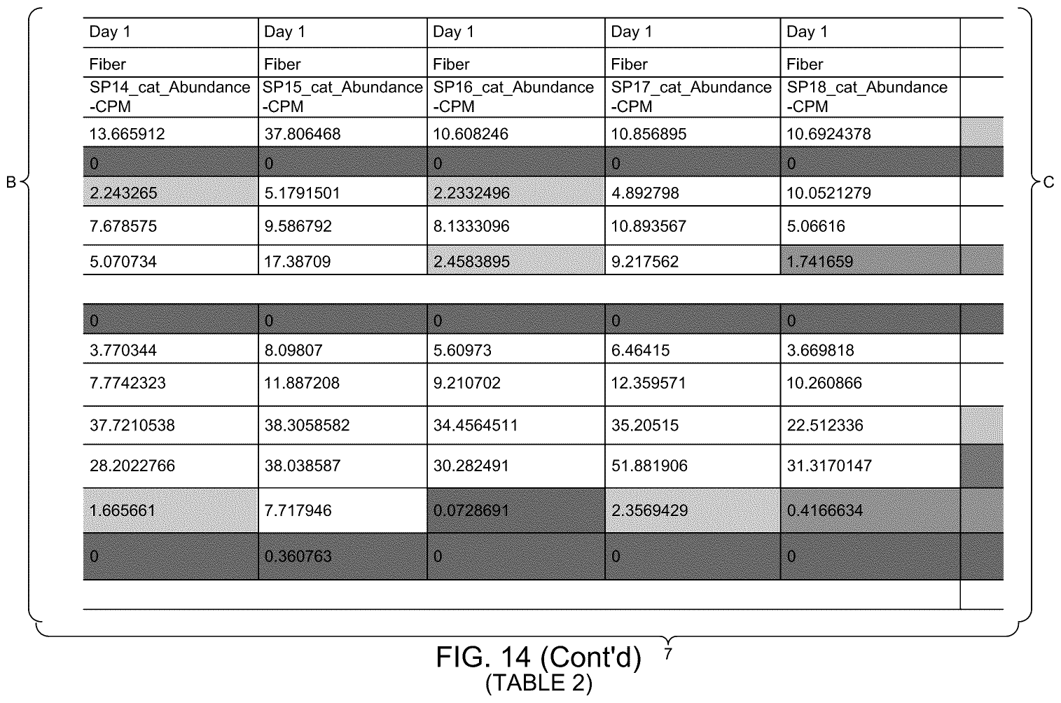

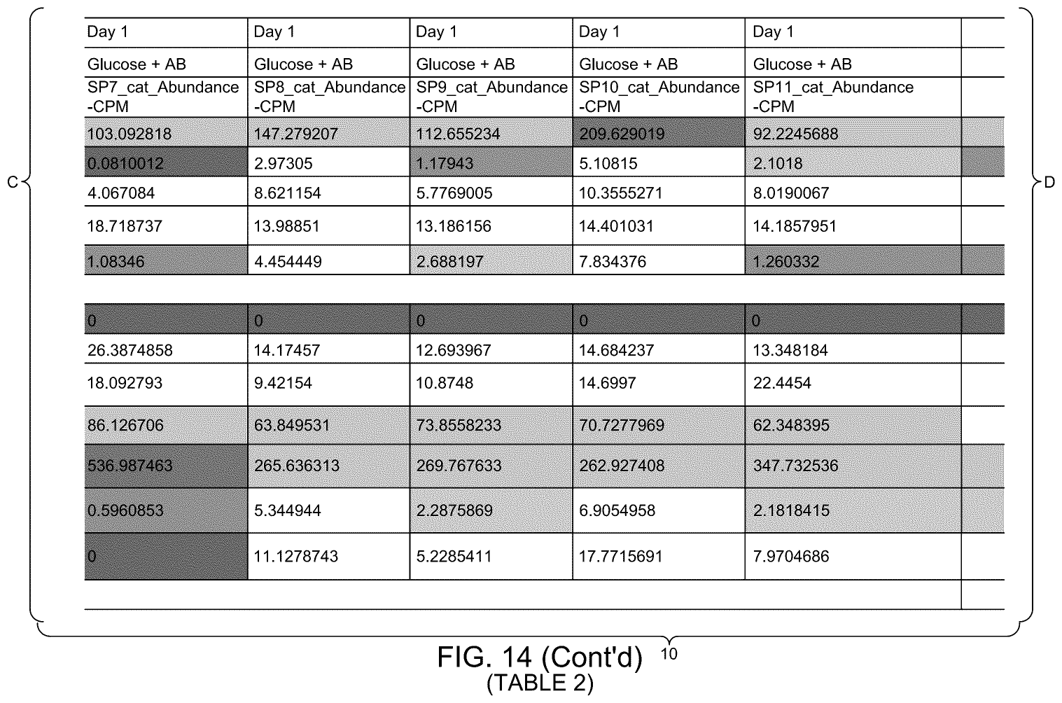

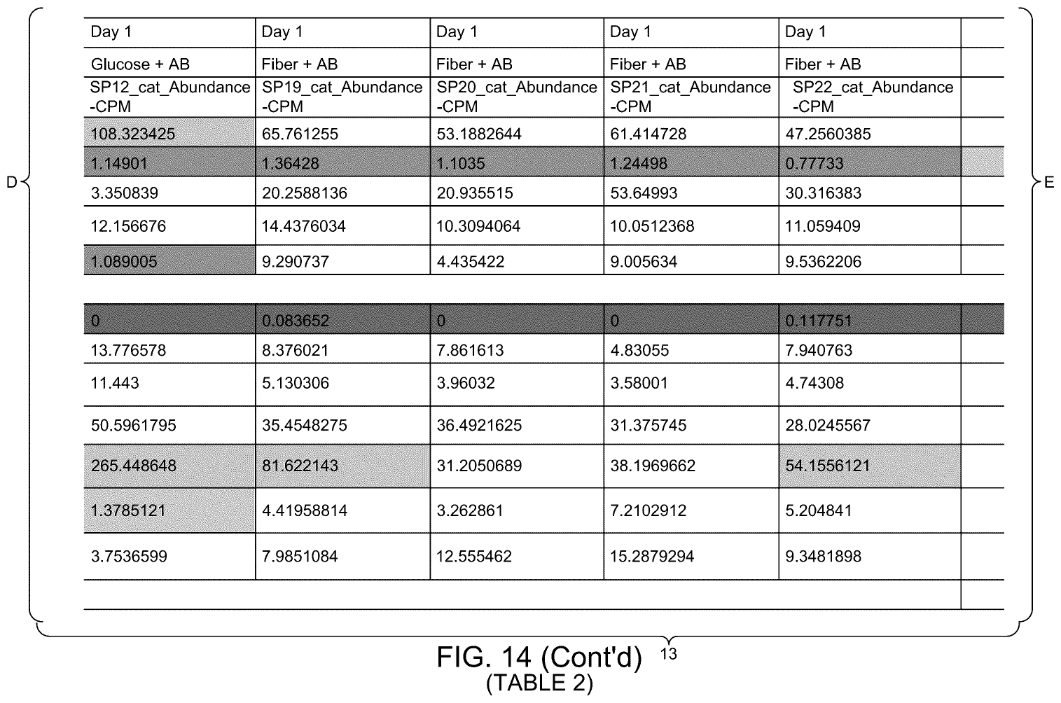

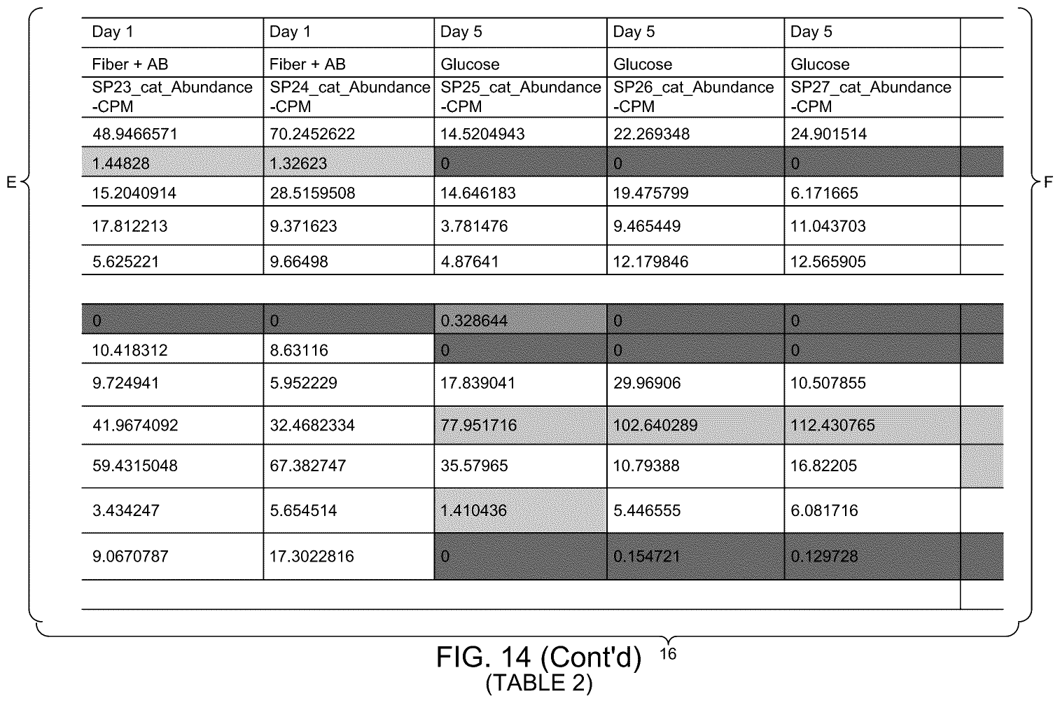

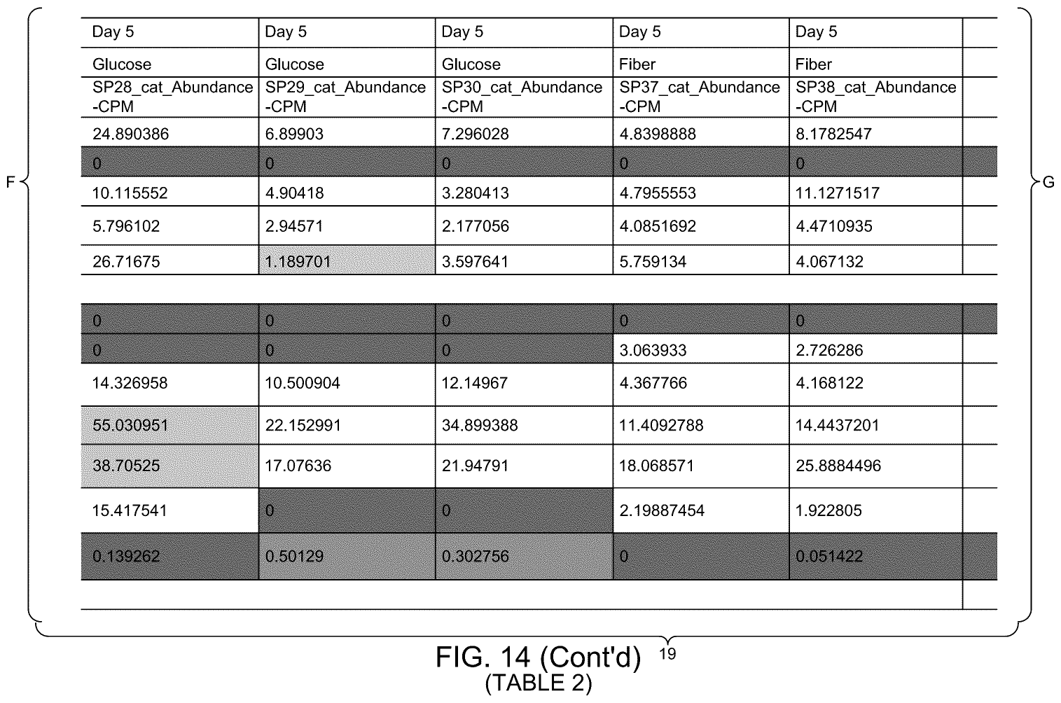

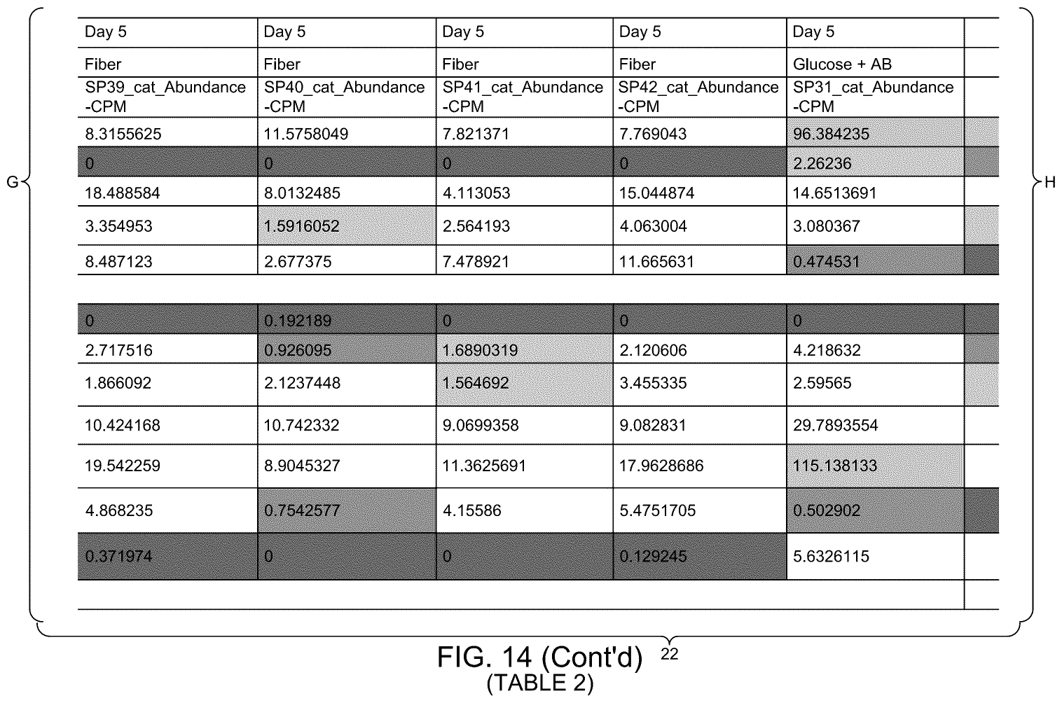

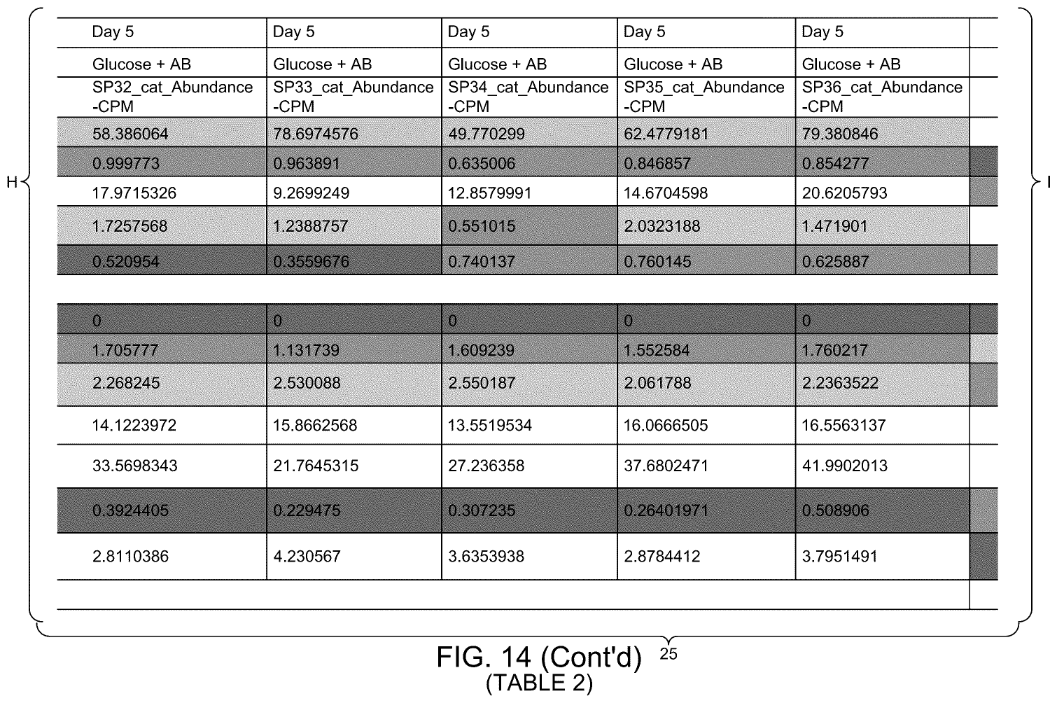

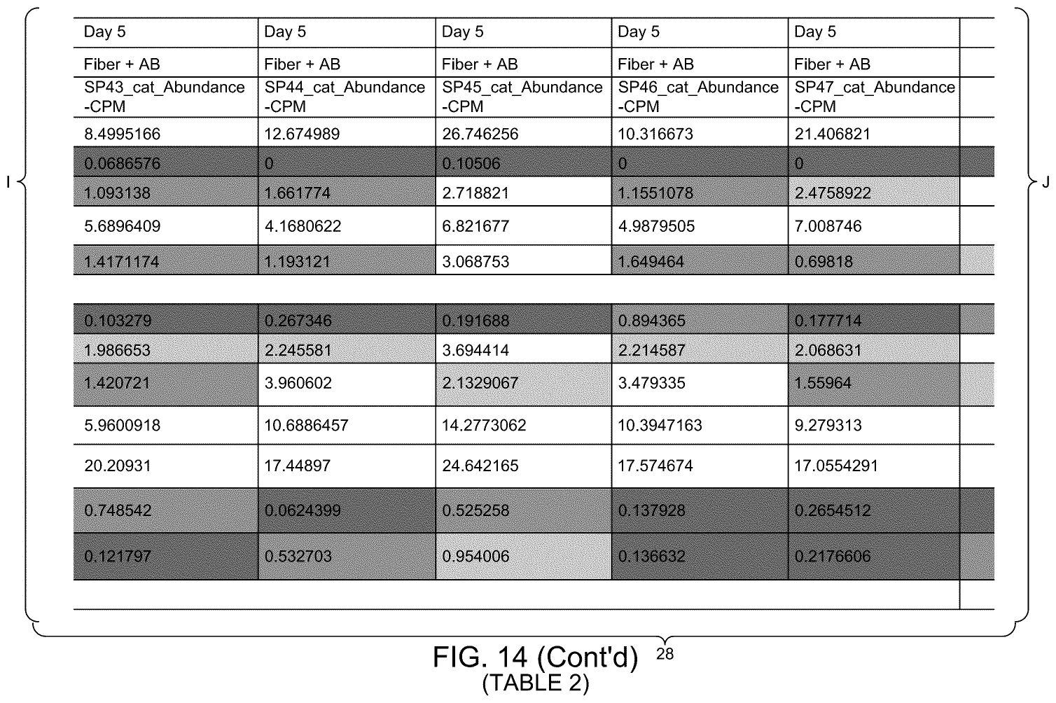

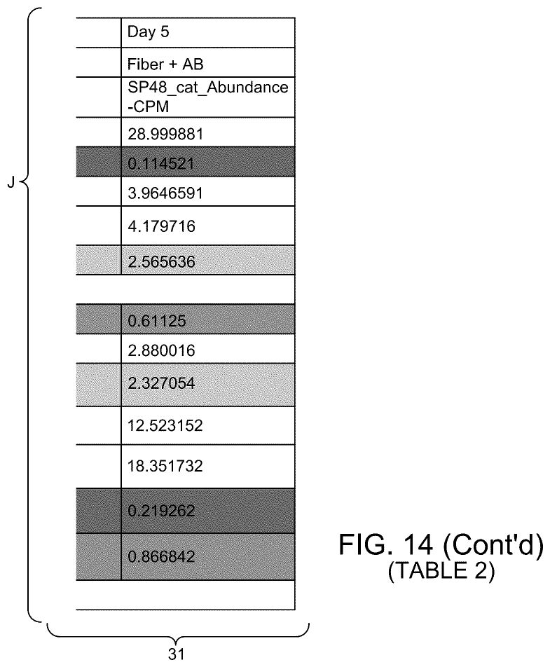

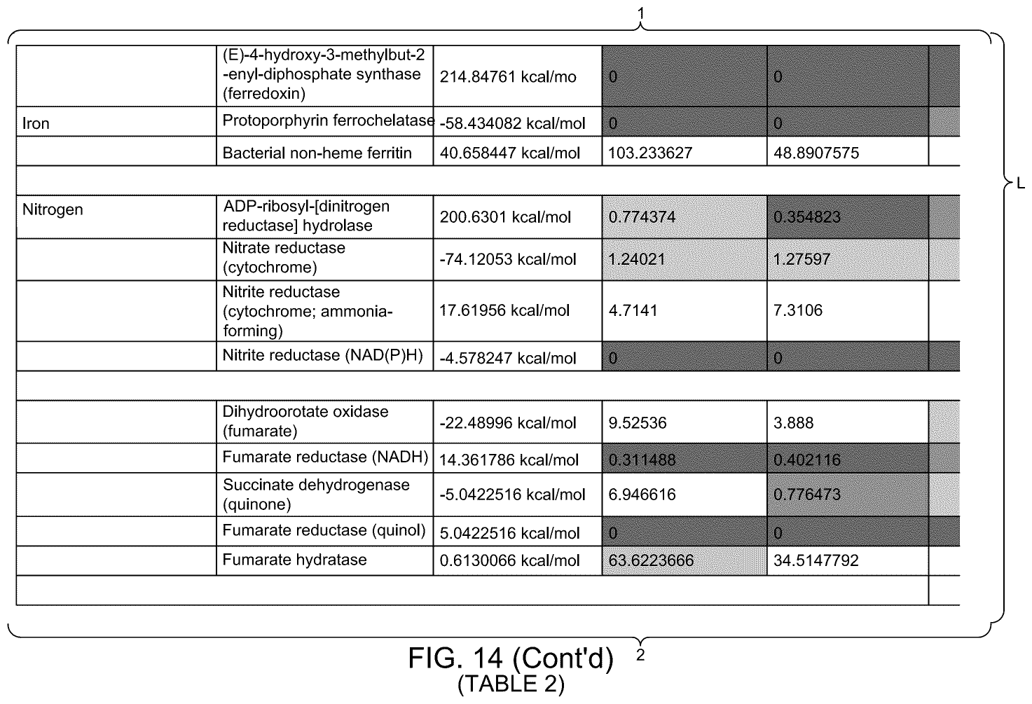

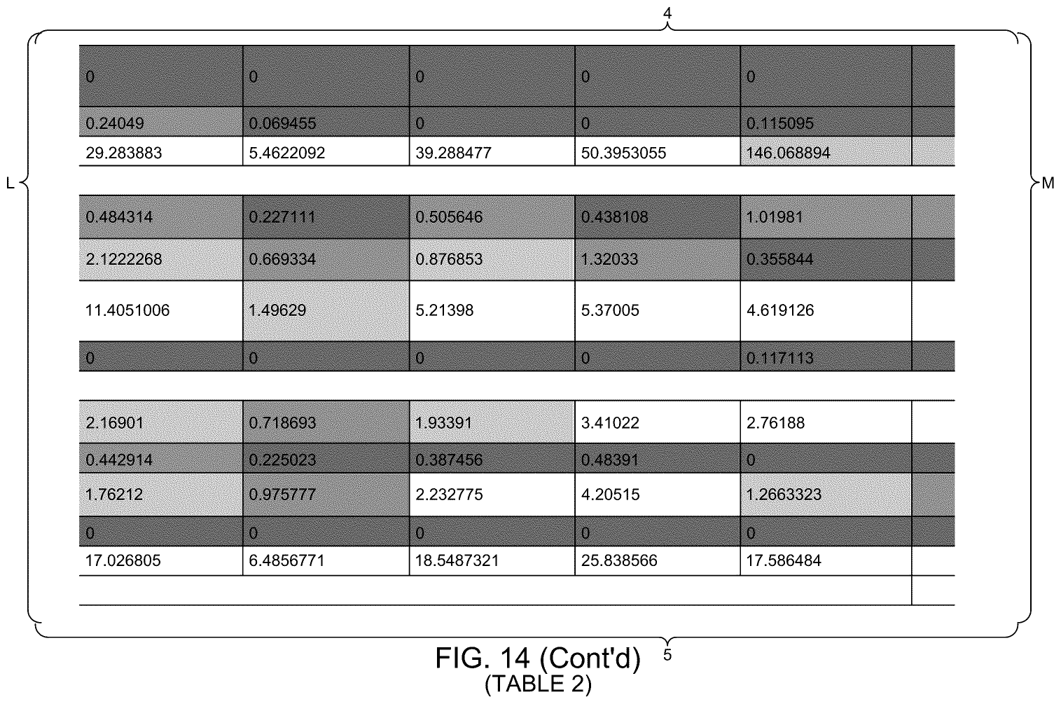

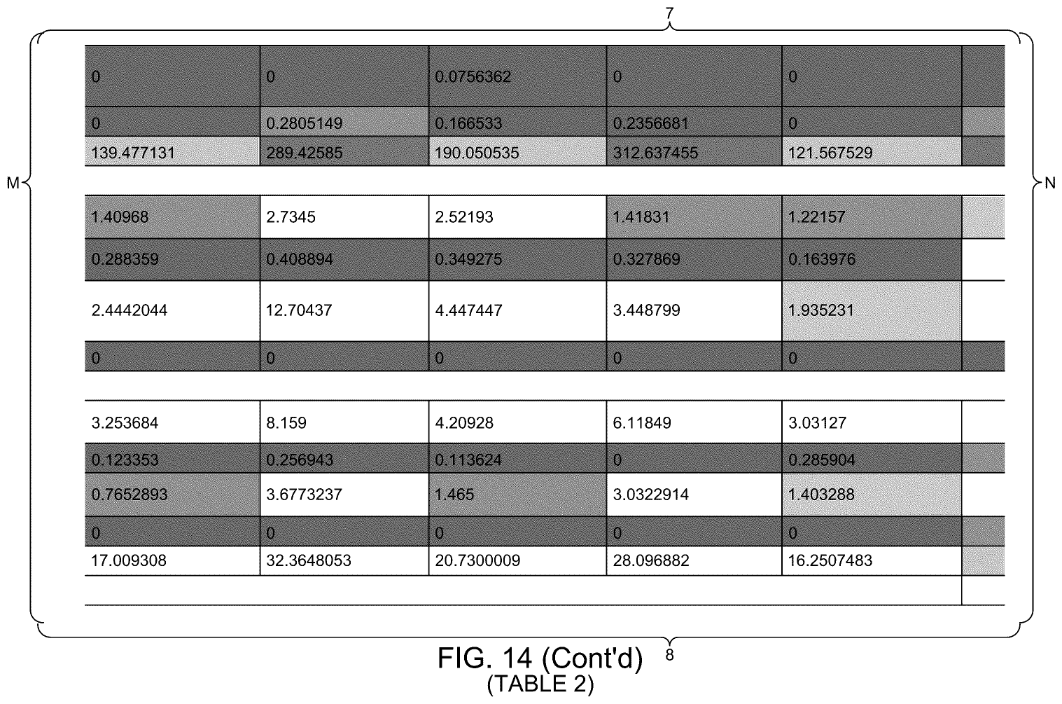

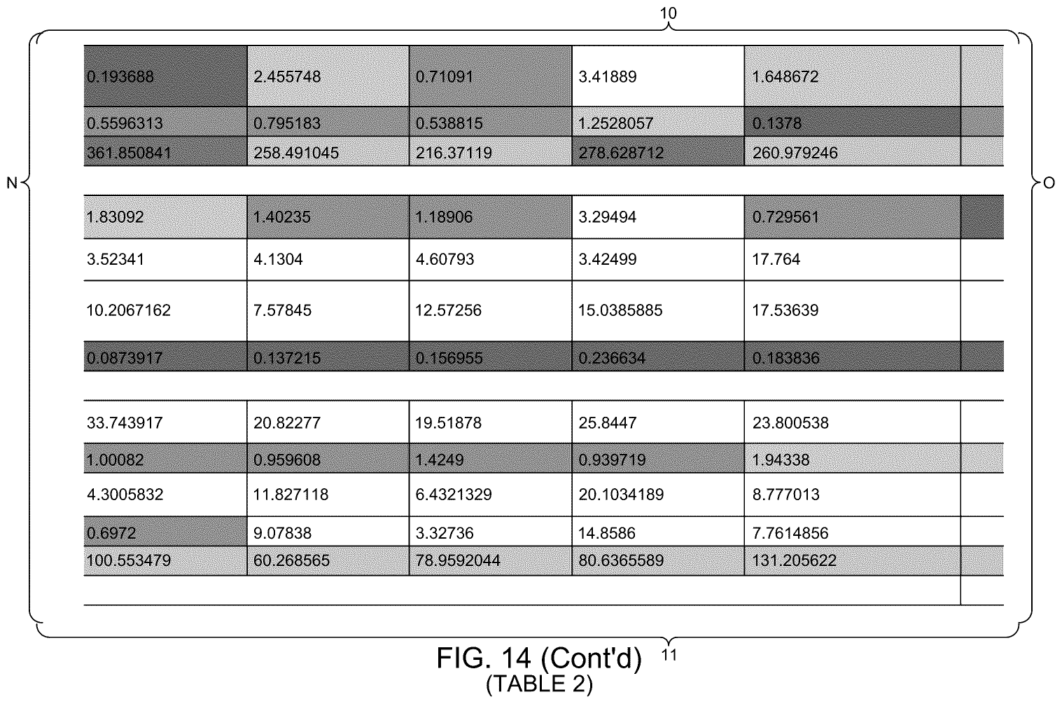

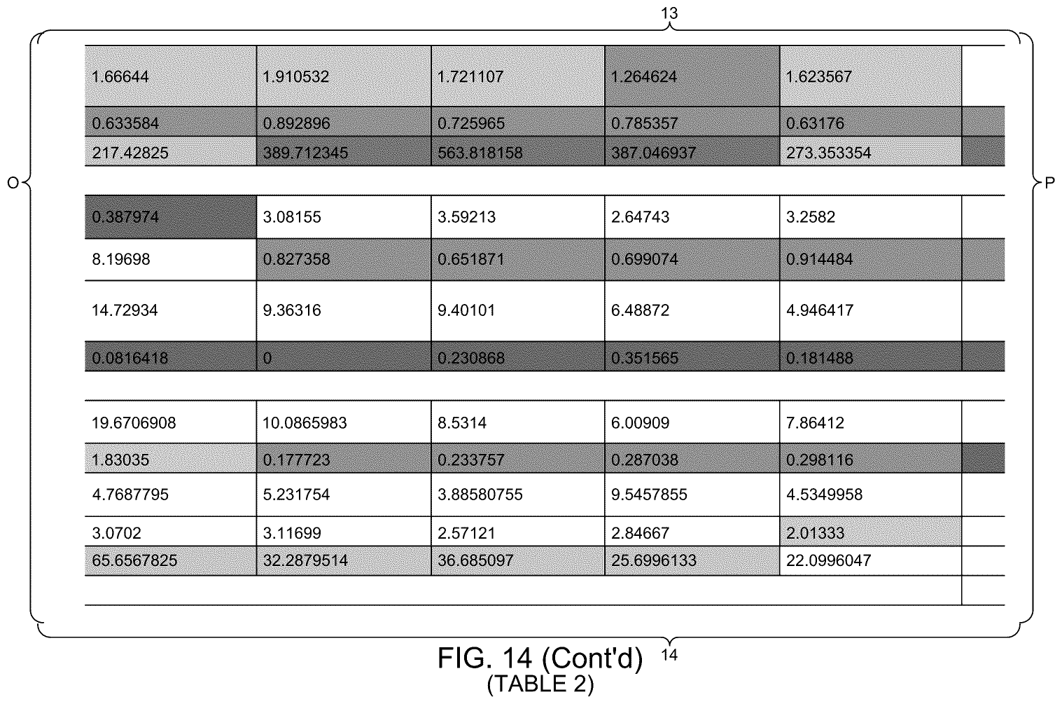

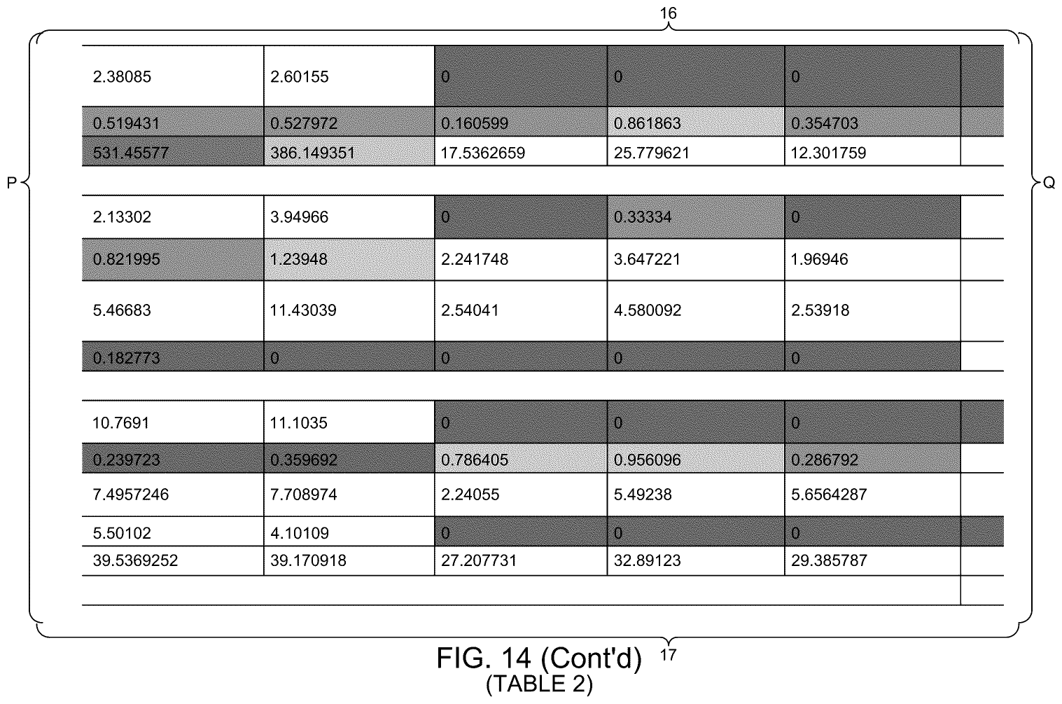

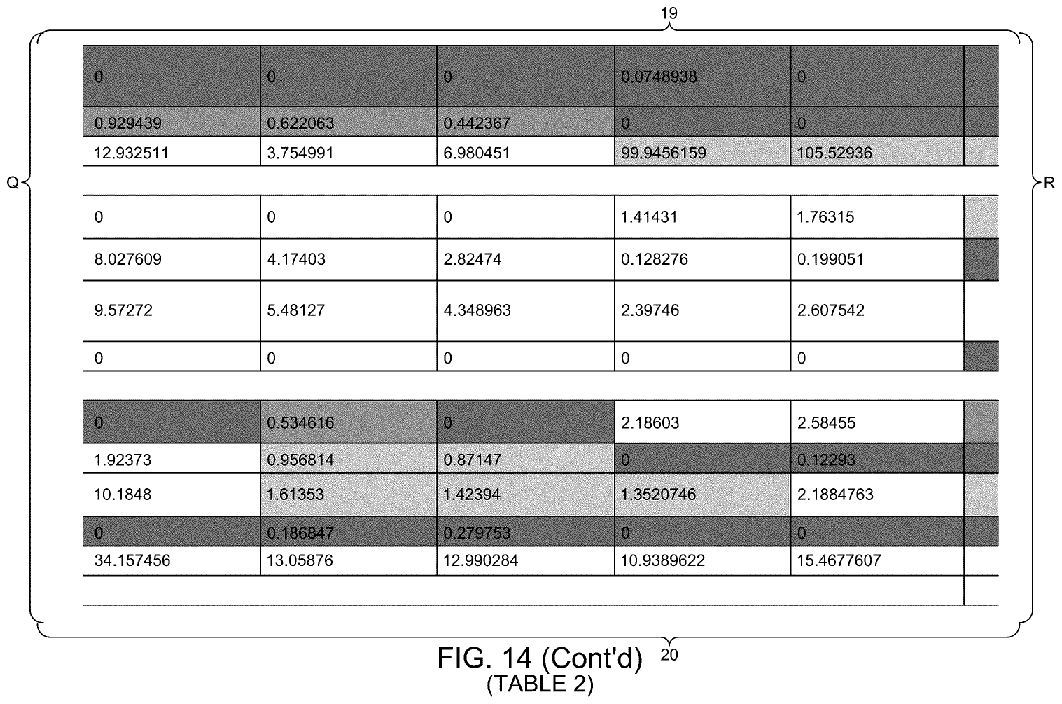

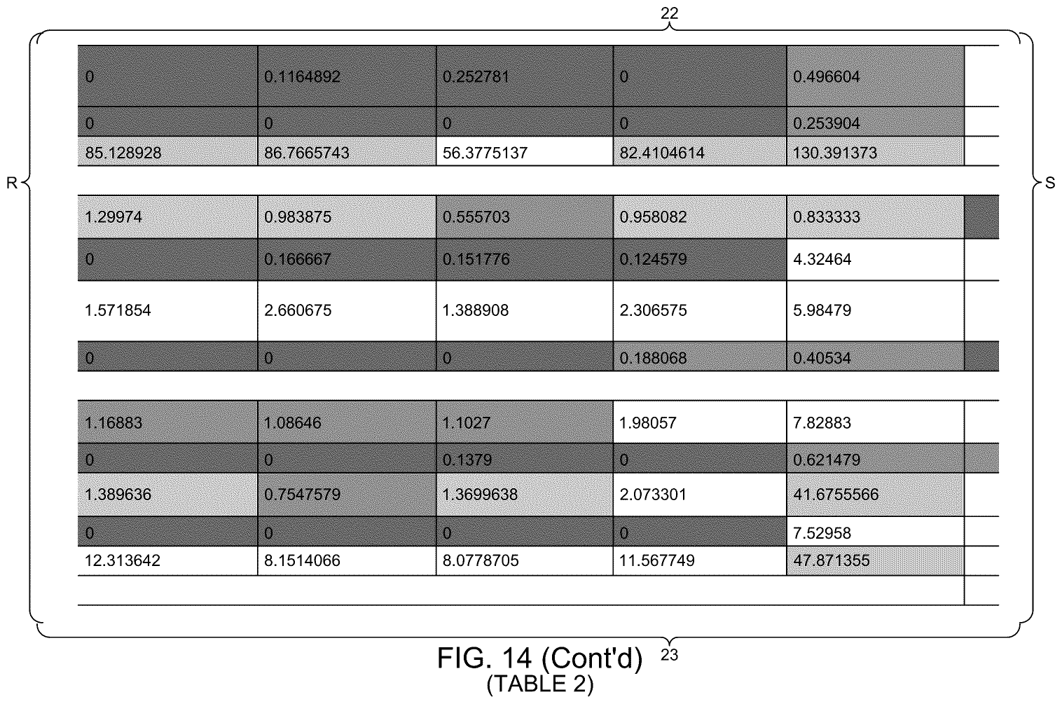

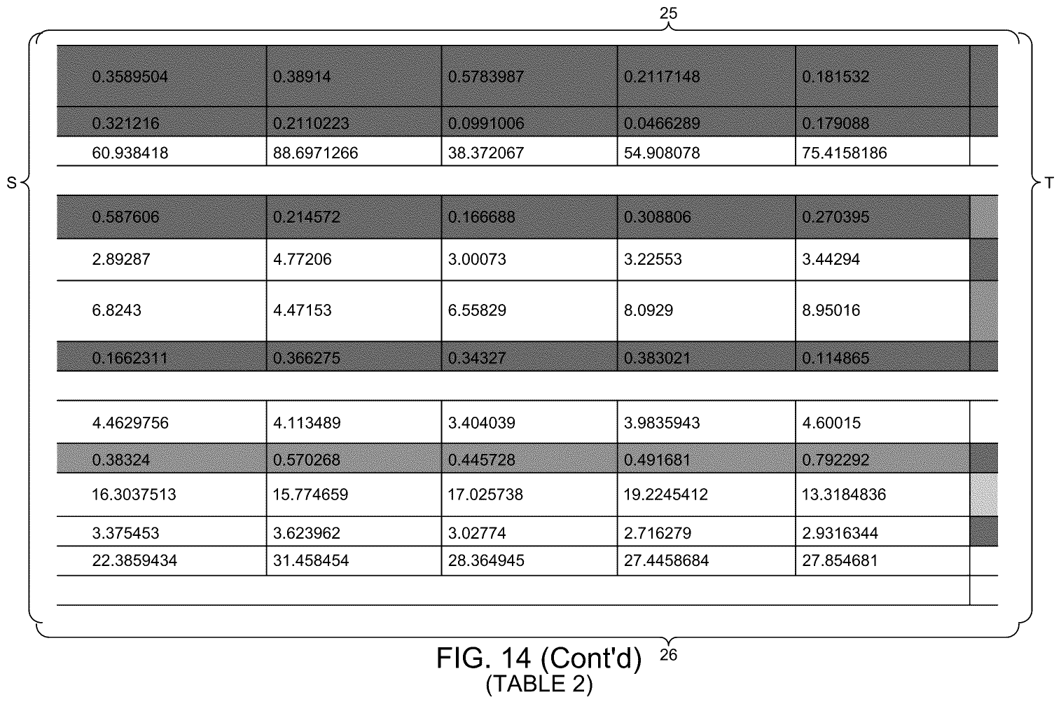

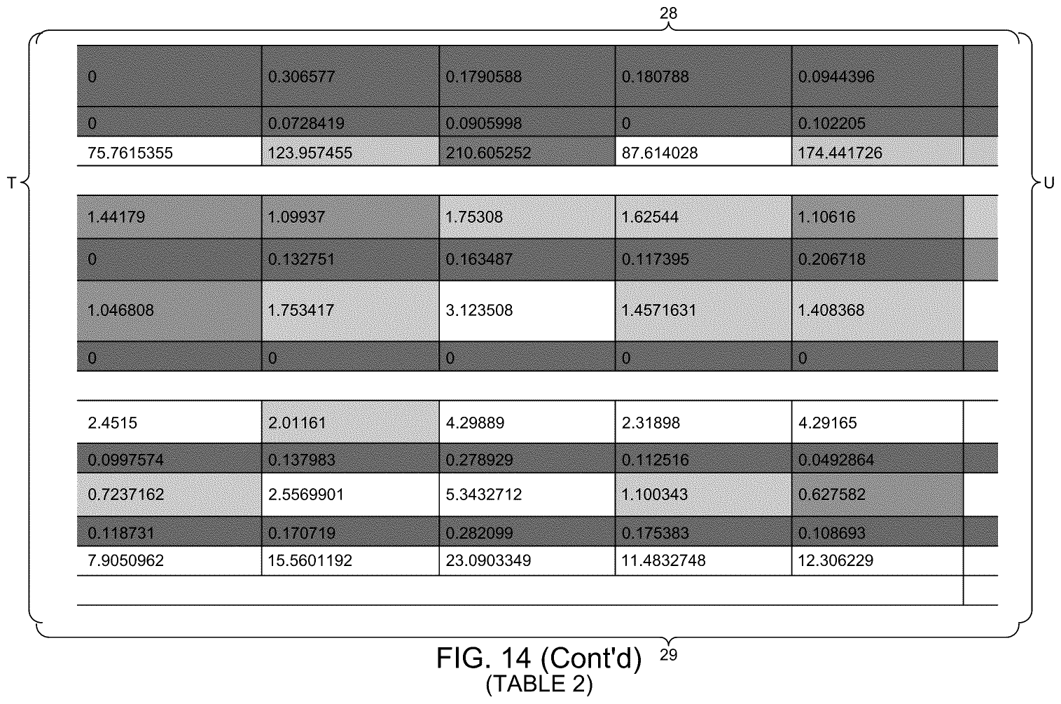

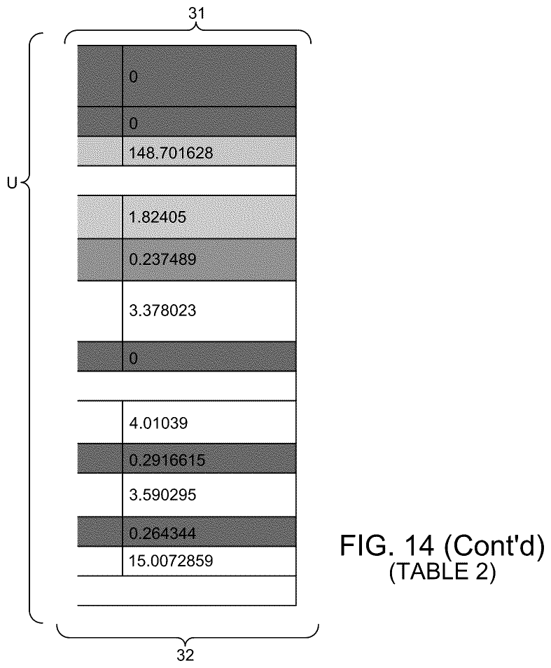

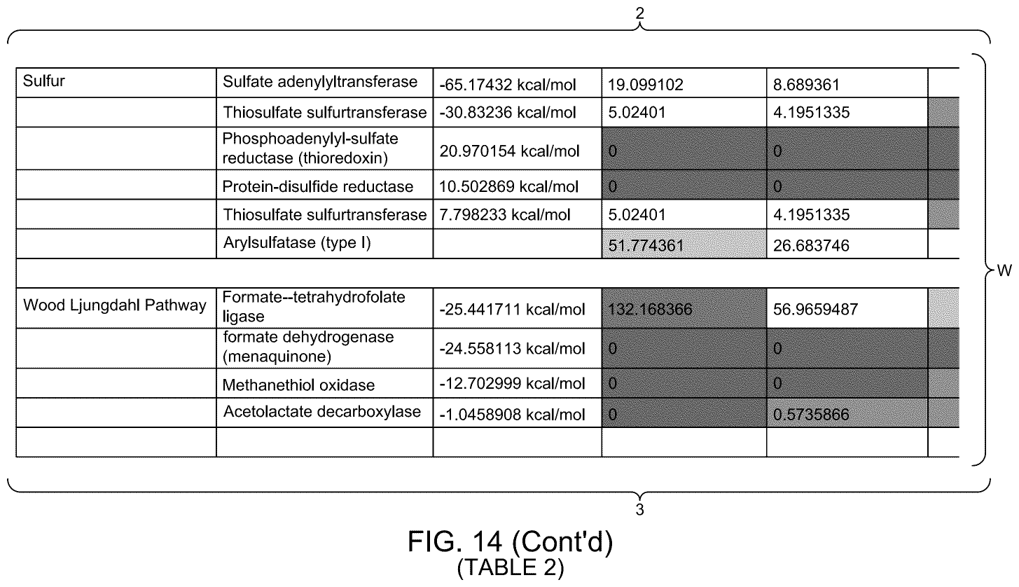

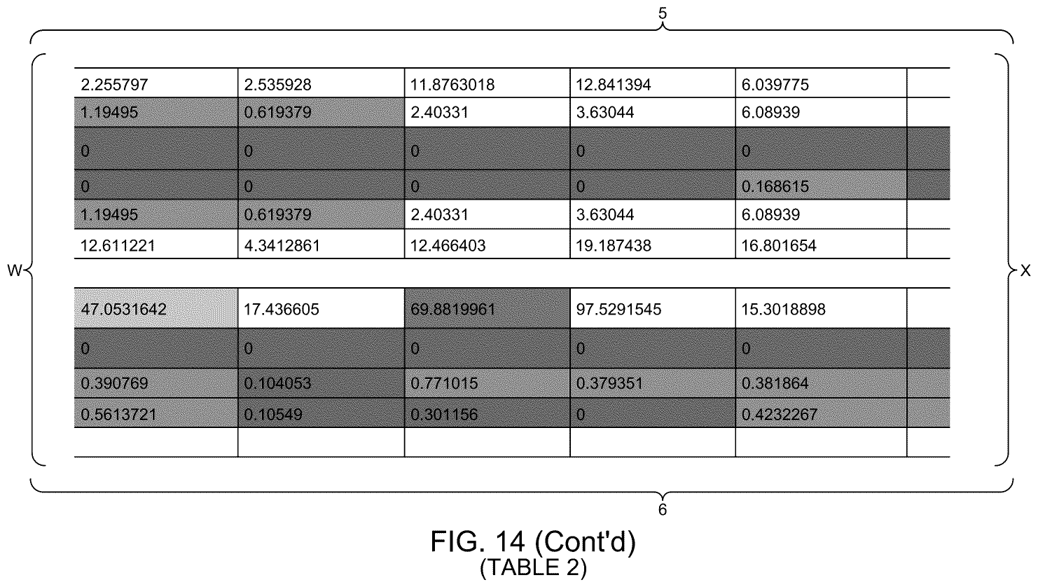

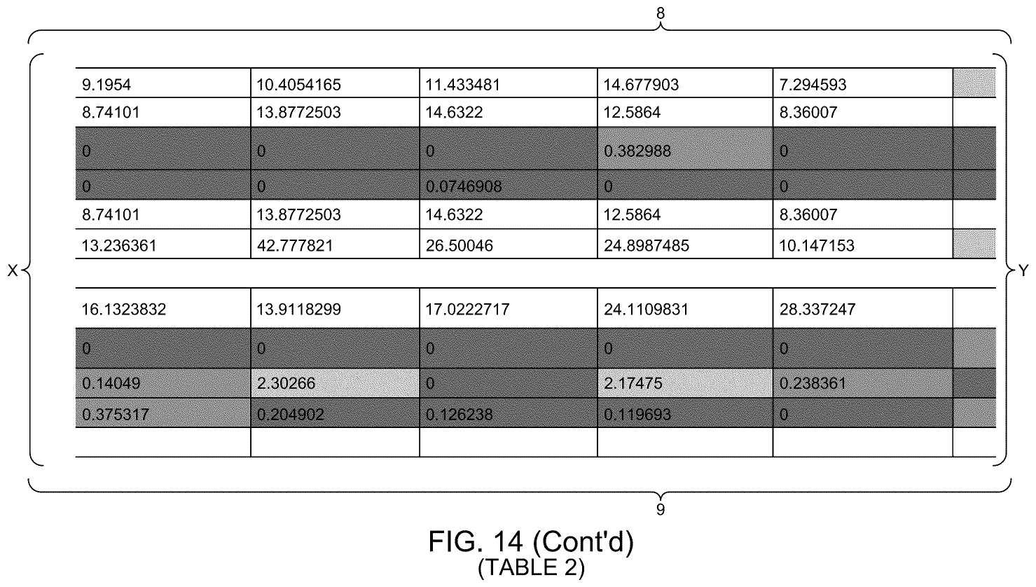

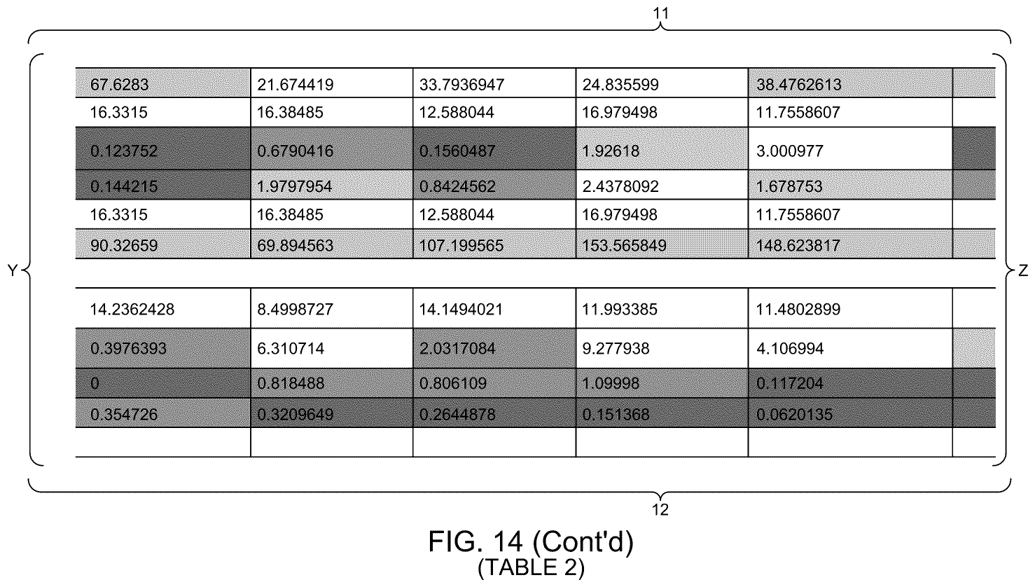

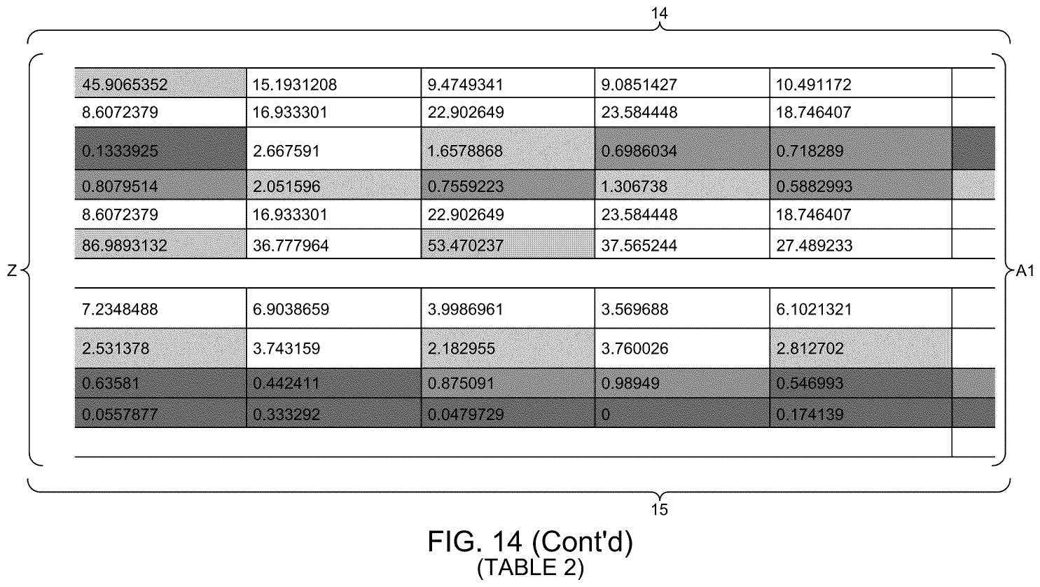

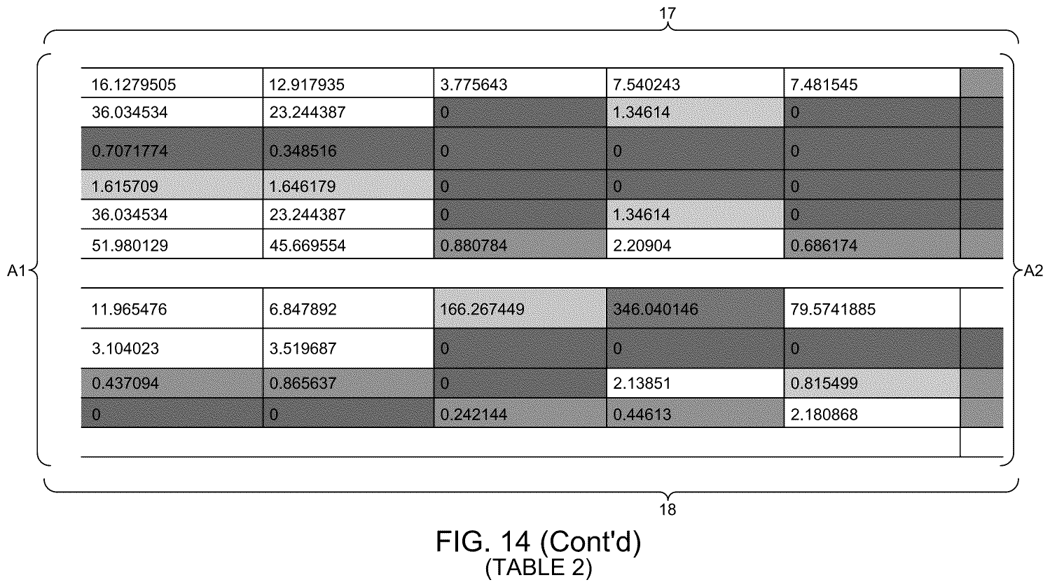

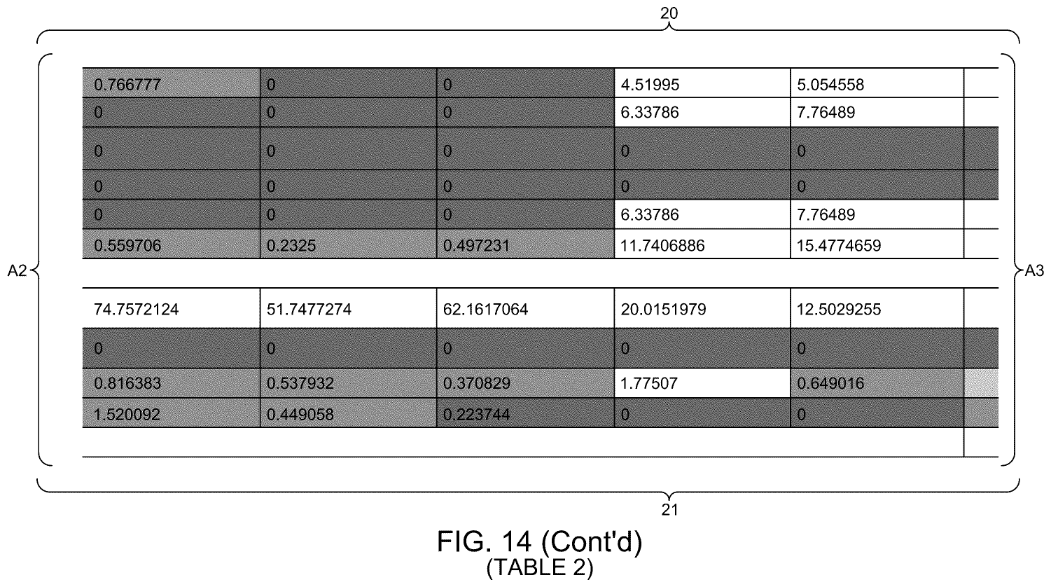

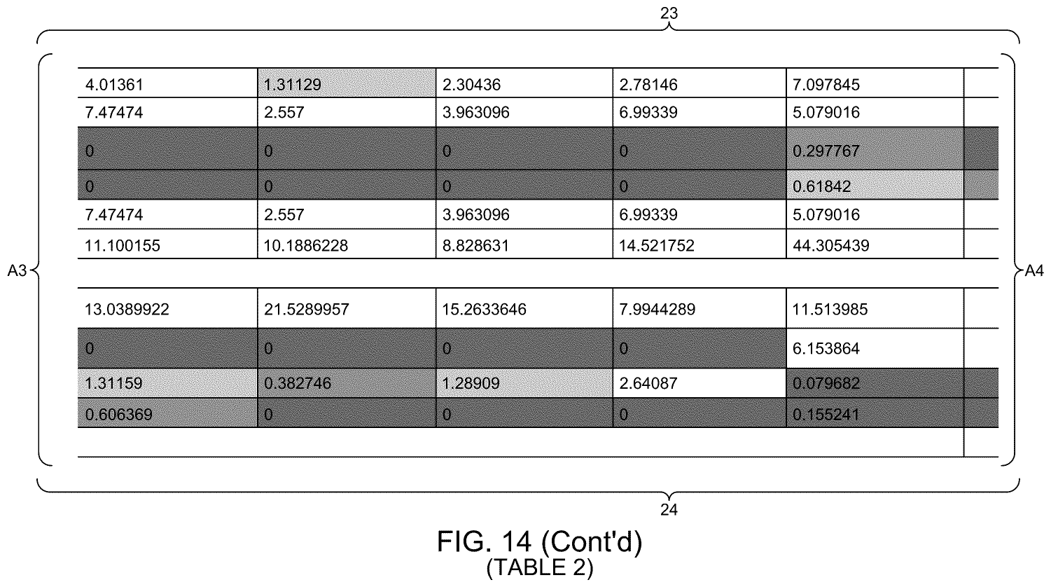

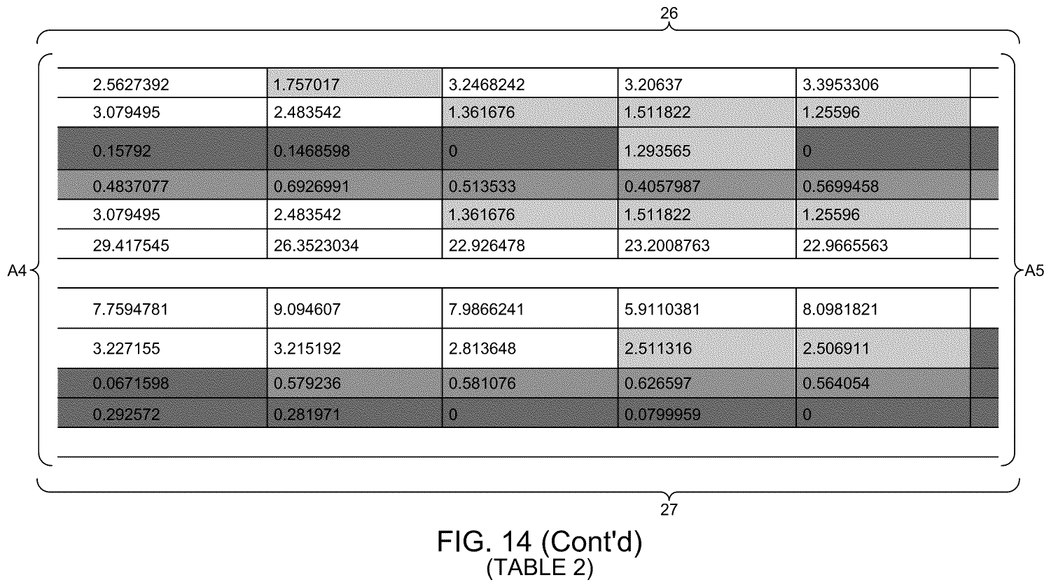

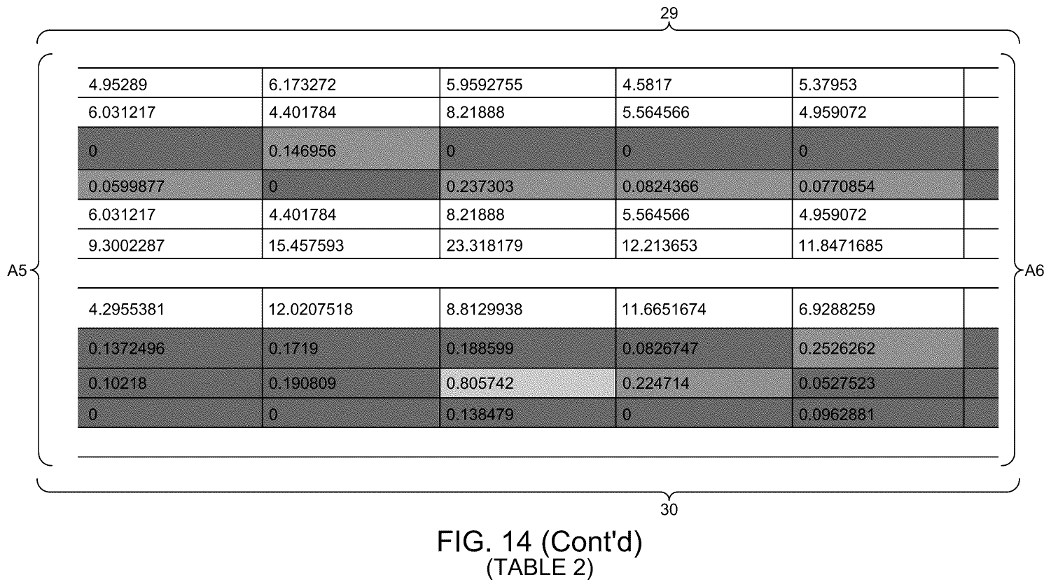

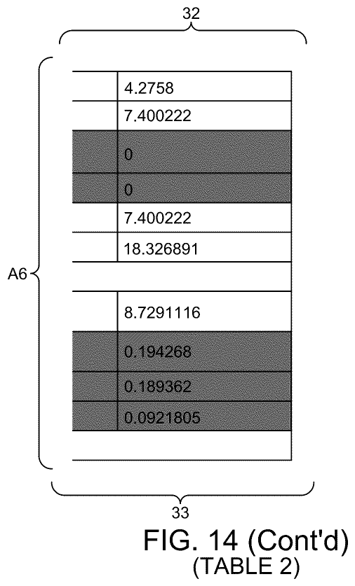

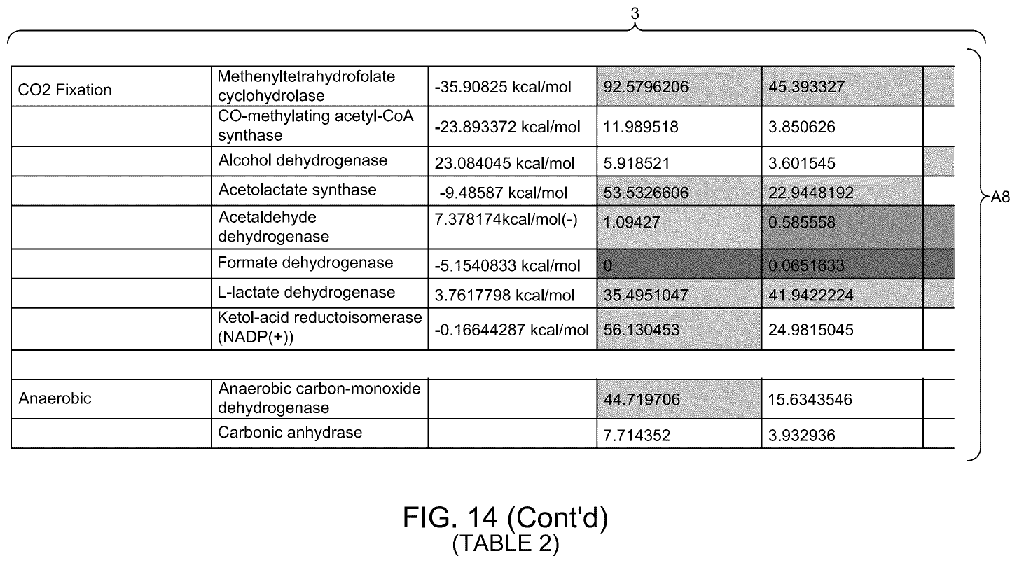

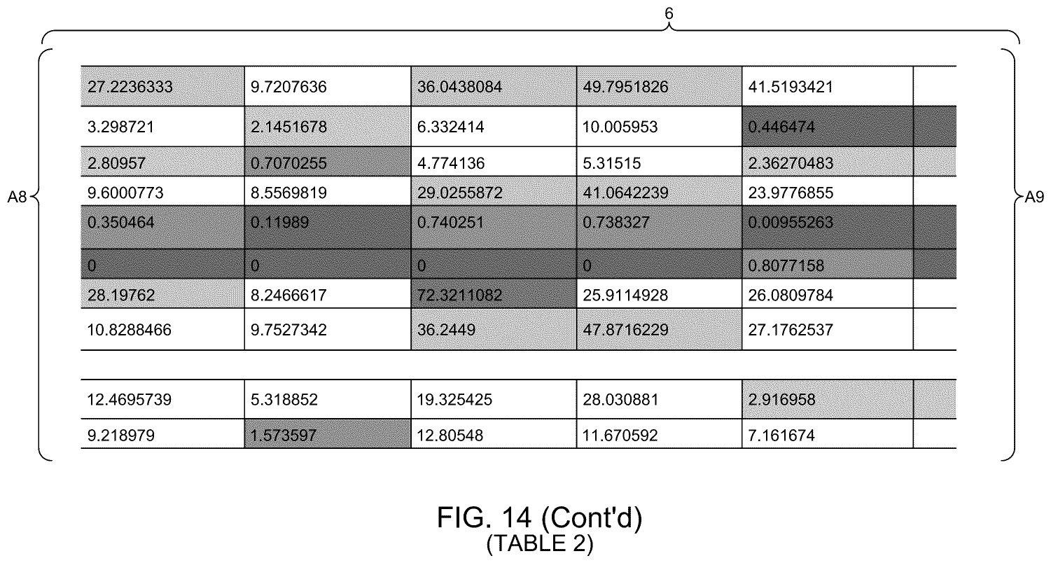

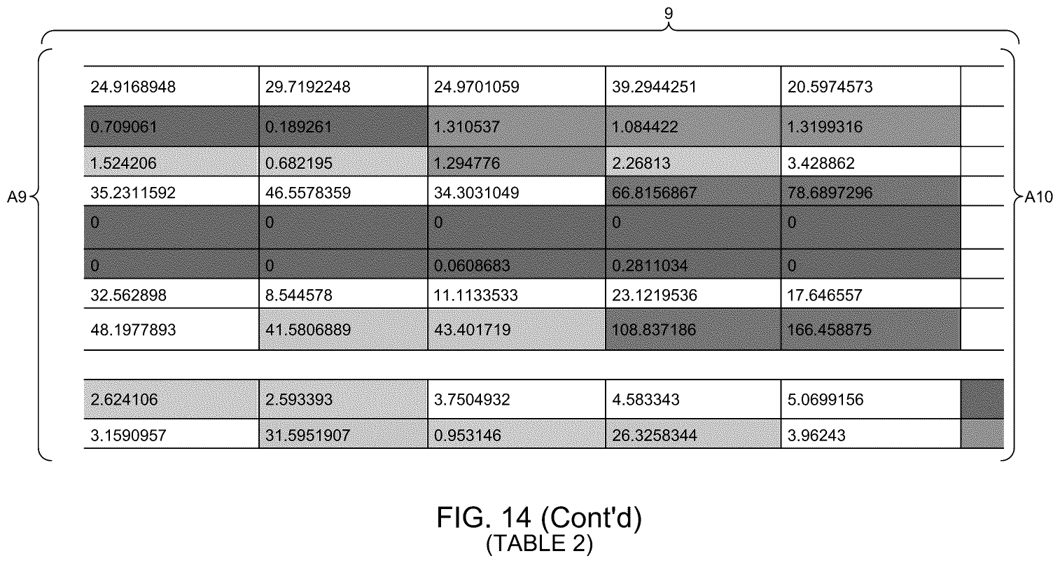

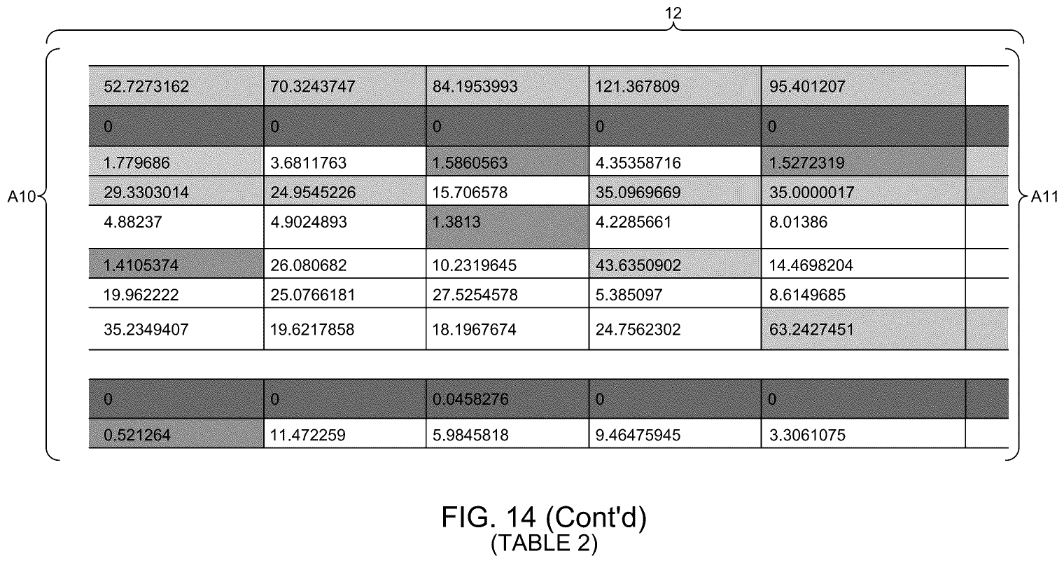

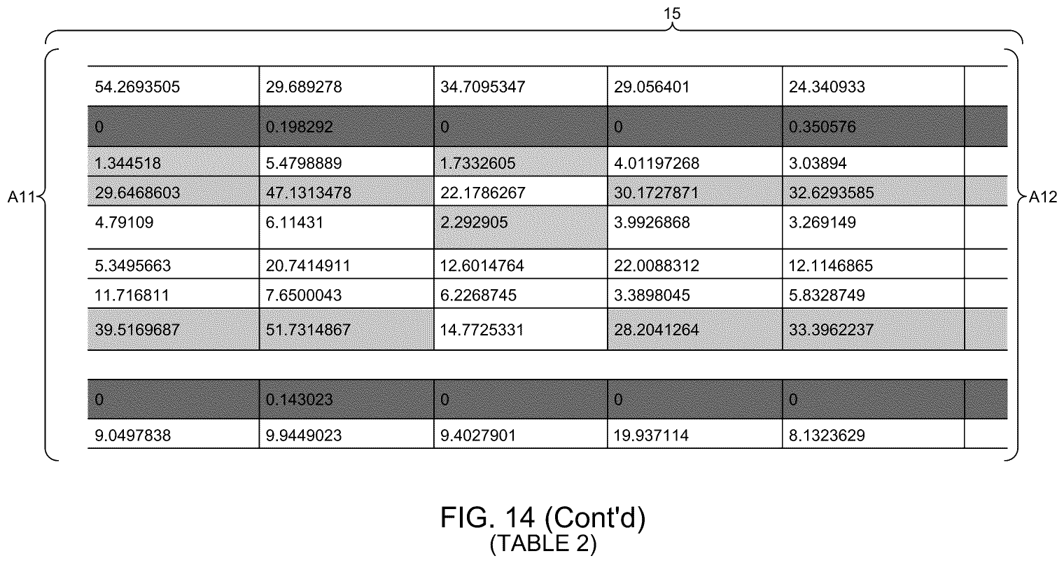

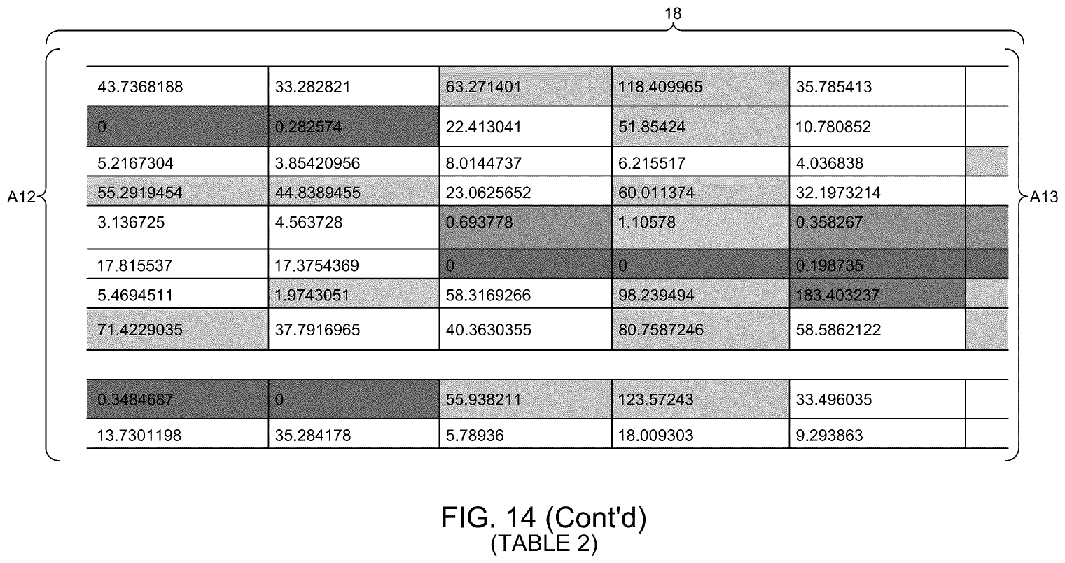

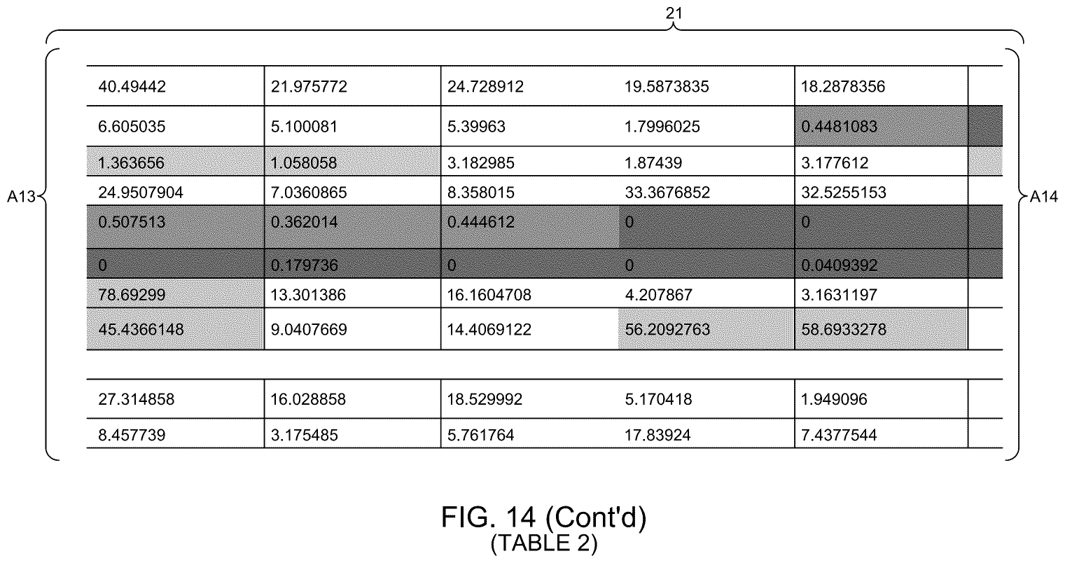

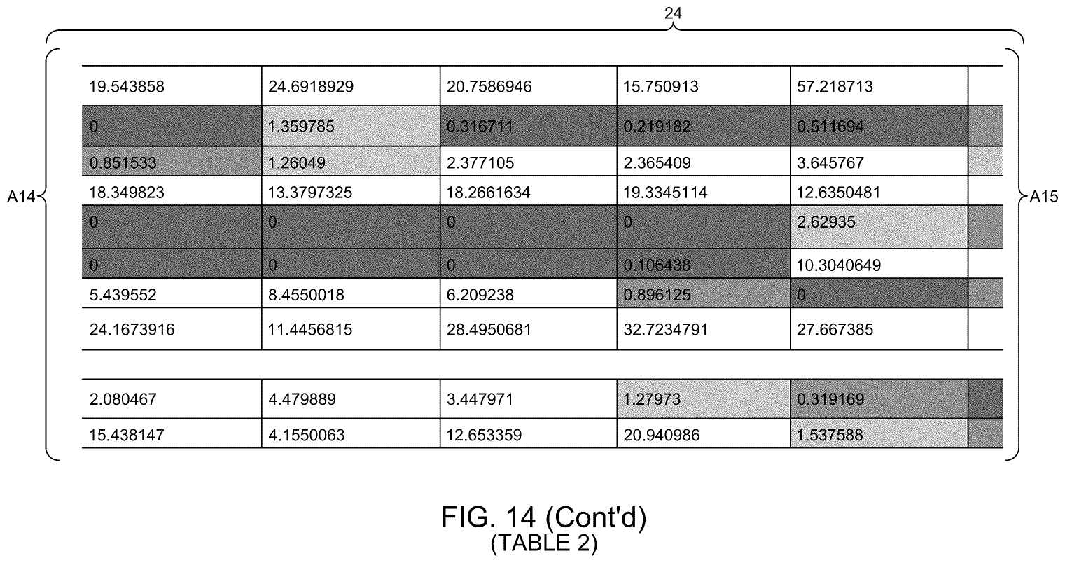

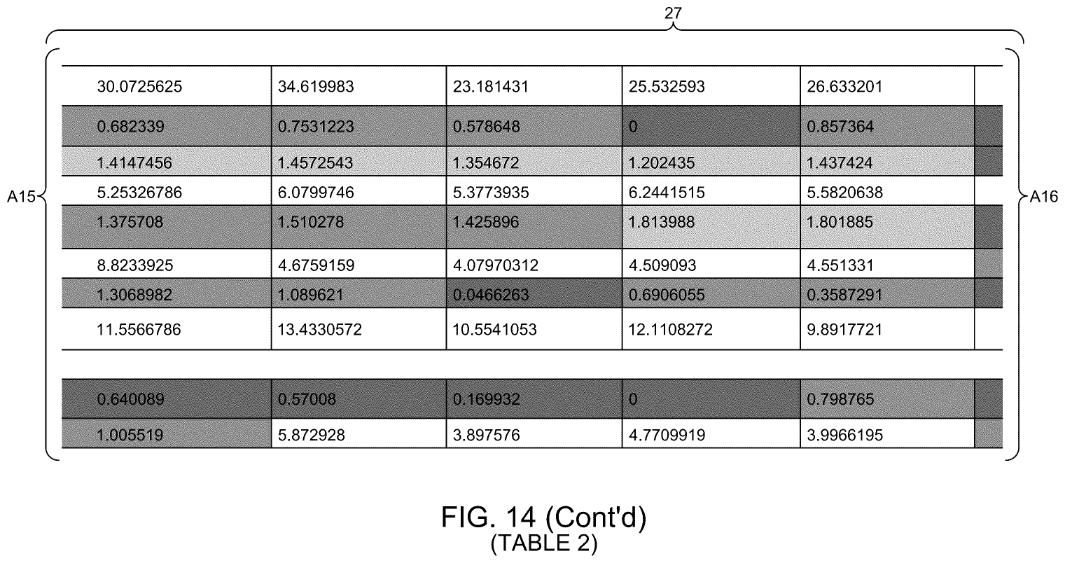

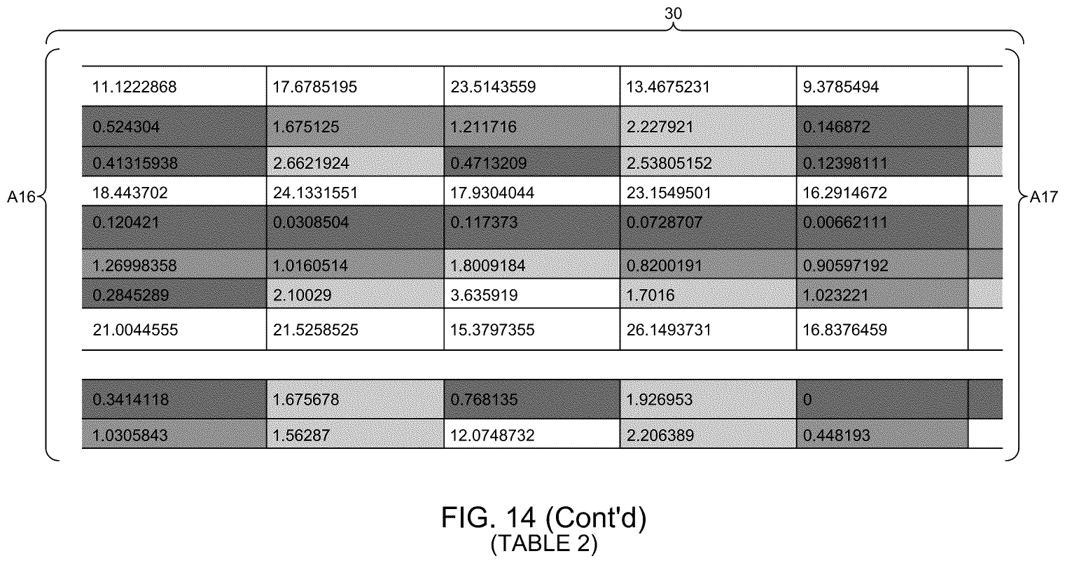

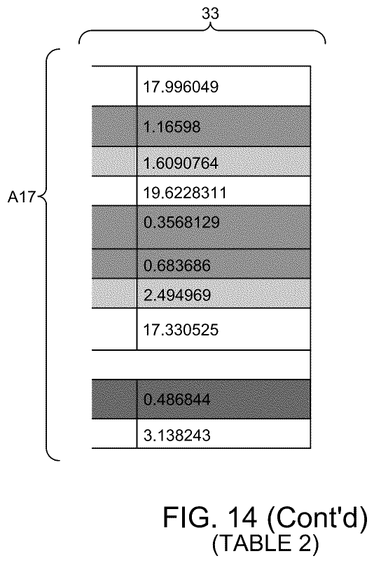

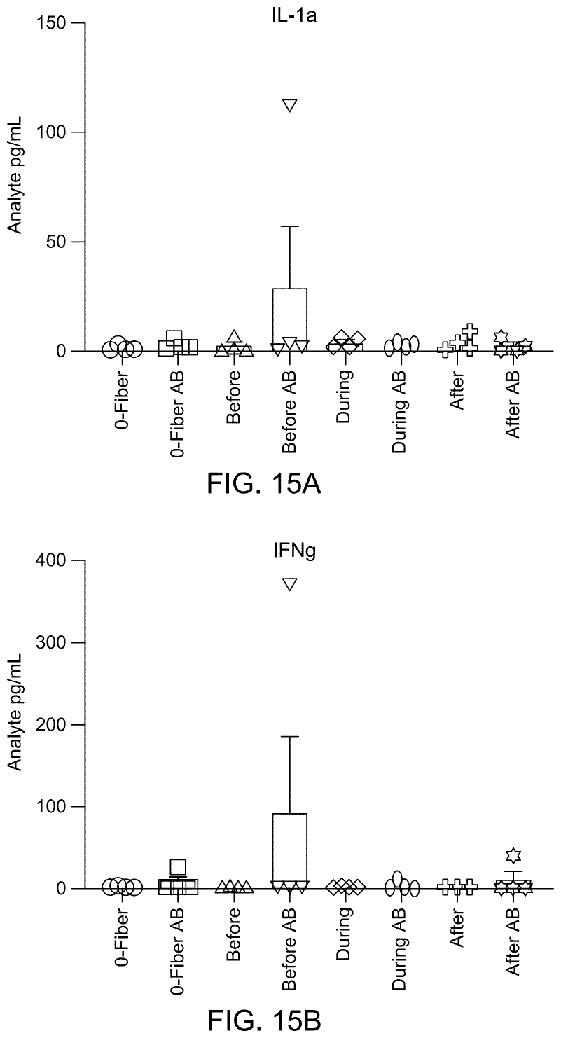

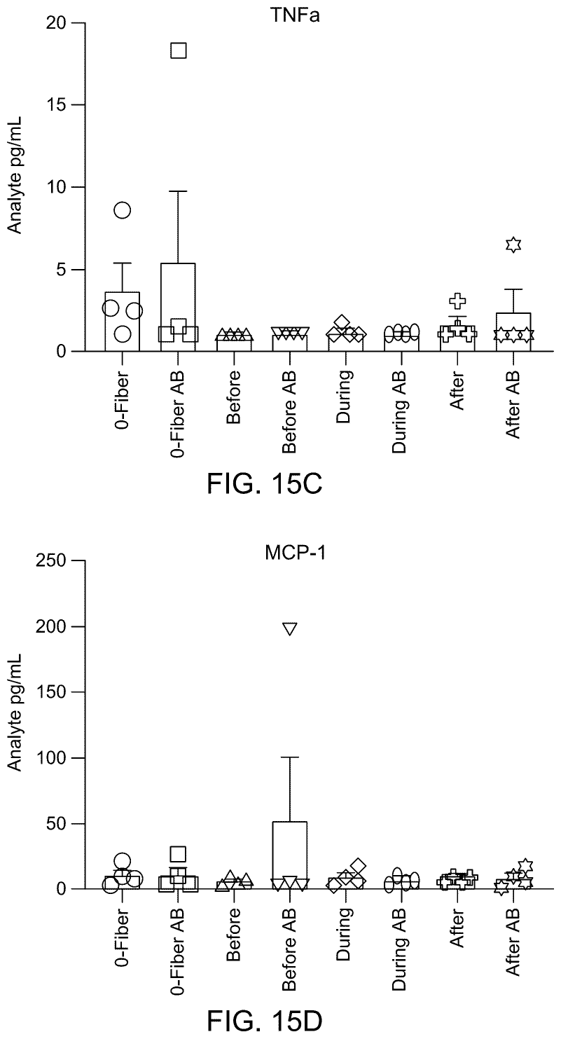

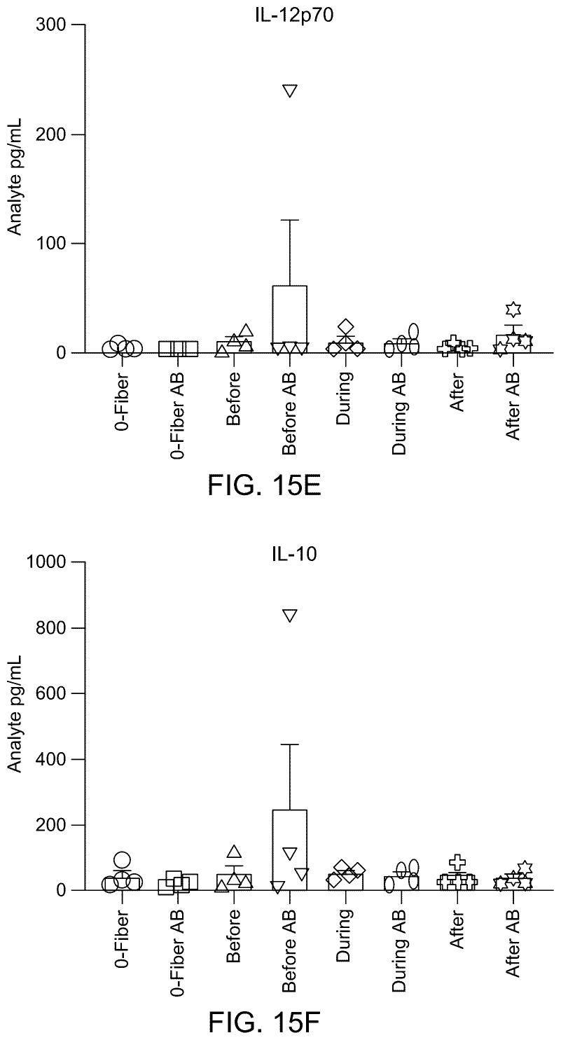

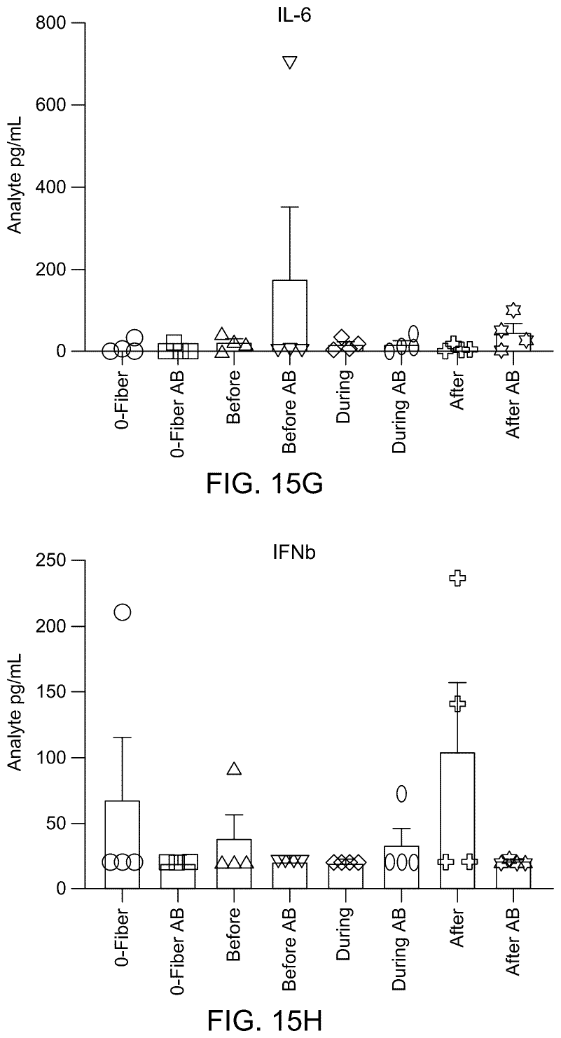

This result shows that the effects of antibiotics on the gut microbiome are dampened by metformin administration. The inventors observed similar effects of metformin on mice fed the laboratory chow, with the metformin treated group displaying smaller reduction in microbial diversity during antibiotic treatment. FIG. 11A-11G.

### Example 7

### Metformin Reduces Initial Increase in Proteobacteria Seen During Antibiotic Treatment and Depletes the Microbiome of Desulfobacterota and Cyanobacteria

Metformin treatment led to several significant changes in the microbial composition of the gut during antibiotic-treatment. In mice on the glucose low-fiber diet, metformin treatment prevented the initial spike in Proteobacteria observed in the untreated group. This lack of increase in Proteobacteria could be due to metformin inhibiting these bacteria from efficiently respiring and blooming in the high-redox environment during antibiotic treatment. The expansion of Proteobacteria seen with antibiotics could be due to their improved fitness in an aerobic high-redox environment and this advantage is removed when metformin is introduced, limiting metabolic capacity and the increase seen in abundance.

Because of the diversity of biochemical activity in the gut, introduction of this drug that affects electron carriers led to other changes in microbial composition. By Day 5 of the assay helping to expand bacteria in the Proteobacteria phyla and a complete depletion of the Desulfobacterota and Cyanobacteria phyla.

The inventors observed the families of Proteobacteria blooming at Day 1 versus Day 5. Different selection pressures may drive the community composition. At Day 1 of the assay, antibiotics led to a bloom in Sutterellaceae and Enterobacteraceae in the glucose diet. This bloom was not present in the metformin and antibiotic treated group. At Day 5 of the assay the metformin antibiotic group had a bloom in Proteobacteria in the Moraxellaceae family. The control metformin group had an increase in Sutterellaceae and Enterobacteraceae.

The Moraxellaceae family has extensive siderophores scavenging iron. See Furano et al. (2005). This siderophore activity might increase the fitness of Moraxellaceae in the iron limitation conditions caused by long-term metformin treatment.

These data show that iron limitation via metformin is potentially driving community composition by Day 5, leading to a bloom in bacteria that can best scavenge iron.

Desulfobacterota and Cyanobacteria prefer anaerobic carbon-fixation pathways catalyzed by iron-sulfur clusters. See Hu & Rzymski (2022). The complete depletion of these phyla in all metformin treatment group in the low-fiber glucose diet and laboratory chow shows that the iron-chelation activity of metformin could lead to broad metabolic inhibition in the microbiome by reducing activity of electron carriers in aerobic and anaerobic environments.

Desulfobacterota and Cyanobacteria that bloom after antibiotic exposure have a known dependence of iron-sulfur cluster activity. See Yamamoto & Takai (2011); Jordan et al. (2021); Hu & Rzymski (2022). The complete depletion of these phyla in all metformin treatment groups shows that the iron-chelation activity of metformin should lead to broad metabolic inhibition.

The inhibition of iron-sensitive phyla by metformin may also explain the lower baseline microbiome diversity observed five days post metformin treatment. This result shows that metformin may have multifaceted effects on the microbiome involving iron limitation, ETC activity and redox potential.

The reduction seen in antibiotic induced Proteobacteria bloom is a potential benefit of metformin supplementation.

### Example 8

### Discussion

The inventors took a multi-omic approach combined with chemical measurements. They first used shotgun metagenomics with de novo gene assembly to give accurate quantitative insights into the microbial ecology. They saw that antibiotic treatment in mice fed a glucose diet was associated with a greater decrease in community diversity and increases in inflammatory phyla such as Proteobacteria compared to mice given a fiber diet. See FIG. 2A-2D. Bacteria in the gut affect host health by their functional activity rather than the presence or absence of a single microbe.

The inventors also used metatranscriptomics. A glucose diet during antibiotic treatment led to an increase in expression of genes involved in the TCA cycle and electron transport. See FIGS. 3A-3C, 4A-4D, and 5A-5D. Fiber-fed mice did not show this burst in respiratory activity and had increased activity of fiber degradation, fermentation and carbon fixation. See FIGS. 3A-3C, 4A-4D, and 5A-5D. This result shows that mice fed a glucose diet during antibiotic treatment had a large shift in the overall biochemistry of the microbiome toward high-energy electron transfers and aerobic metabolism.

The increase in respiratory metabolism seen in mice given the glucose-diet during antibiotic treatment should lead to the increase in specific bacterial phyla, because they have bacterial machinery required for survival in the antibiotic-induced high redox environment. The inventors searched the metagenome assembled genomes for the largest protein in the respiratory chain, NADH oxidoreductase (complex 1). They measured the change in relative abundance of bacteria containing complex 1 during antibiotic treatment. Mice given a glucose-diet had a significant increase in bacteria with complex 1 from Day 1 to Day 5 of the assay, while the fiber-diet showed a decrease. This result shows that the changes seen in bacterial taxa in the glucose-fed mice given antibiotics may be due to increased fitness in a high redox potential environment. The changes in metabolic activity seen in the metatranscriptomics is corroborated by chemical measurements of eH and pH in the cecum of mice. Antibiotic treatment led to a sharp increase in redox potential (eH) and pH of the gut microbiome in mice given a glucose diet while the mice fed a fiber diet had no significant chemical changes on Day 5 during antibiotic treatment. See FIG. 6A-6F. The inventors then used a pharmaceutical inhibitor of respiratory metabolism—metformin—to potentially disrupt this activity and test the function of respiration on antibiotic susceptibility in vitro and in vivo. Metformin was shown to protect bacterial populations from antibiotics in monoculture and in mice given a metformin supplement on the glucose diet.

Fiber supplementation before during and after antibiotic treatment is protective from AID. The tested stages of supplementation led to fewer changes in the microbial ecology of the gut with improved microbial diversity at the end of the assay compared to the control group. See FIG. 7A-7C. The beneficial effects of fiber supplementation on the antibiotic-treated microbiome were seen even with single purified fibers at 5% supplementation. FIG. 7A-7C.

A result of antibiotic therapy is the marked decrease of obligate anaerobes and an increase in facultative anaerobes (FIG. 2A-2D), which disrupts homeostatic pH and oxygenation gradients in the intestinal tract (dysanaerobiosis). Dysanaerobiosis elicits a cascade of events including increased inflammation, decreased SCFA production, and disruption of intestinal integrity/barrier function. See Jones & Neish (2017); Rivera-Chávez et al. (2017). These associations led to the “oxygen hypothesis”, which states that changes in the anaerobicity of the gut changes the ratio of facultative to obligate anaerobes and leads to dysbiosis and changes in gut redox potential. See Henson & Phalak (2017); Rigottier-Gois (2013); Rivera-Chávez et al. (2017). Antibiotics can increase redox potential in the gut environment, thermodynamically disfavoring fermentative metabolism. See Speijer (2017). A drastic increase in redox potential can further exacerbate gut homeostasis and increase aerobic respiration. These observations suggest that altering bacterial metabolism in the gut microbiome by manipulating diet could affect their response and recovery to antibiotic treatment. Previous attempts at mitigating dysbiosis primarily focused on using probiotics. See de Gunzburg et al. (2018). Publications report that the use of probiotics in a depleted microbial environment helps to expand probiotic species, which are not natively present at a high abundance. Suez et al. (2018).

Using dietary fiber acts as a prebiotic to the gut microbiome, can alleviate AID, and should be able to be a therapeutic alternative to probiotics.

### Example 9

### Diet and Redox Potential and the Gut Microbiome

In the assays, the inventors used a glucose-supplemented diet as the baseline control for the protective high-fiber diet. Glucose is easily absorbed in the upper gastrointestinal tract. A diet high in glucose and low in fiber could lead to carbon limitation in the gut microbiome. A low-fiber diet is harmful to the microbiome by selecting for species that can forage alternative carbon sources for energy, often originating from intestinal tissue. See Ng et al. (2019). Fiber is host-inaccessible and passes to the large-intestine mostly unaltered. Dietary fiber can manipulate bacterial metabolism in the gut by changing the available carbon source. See Carlson et al. (2017). The complex structure of fiber encourages slower metabolic processes in bacteria because it requires extensive protein machinery for its breakdown and uptake (So et al., 2018).

The inventors showed that increasing dietary fiber intake can promote fermentative metabolism and protect native gut anaerobes from antibiotics. See FIG. 3A-5D. The importance of fiber in decreasing dysbiosis has been established in non-antibiotic conditions. Wastyk et al. (2021). The inventors showed that a high-fiber diet metabolically selects for fermentation and carbon fixation, while a low-fiber high glucose diet selects for aerobic oxidative metabolism. Previous publications associated reduced metabolic efficiency in vitro with reduced antibiotic efficacy providing a direct link to the observed protective impacts of fiber. See Cabral et al. (2019).

Using dietary fiber sets up a protective anaerobic environment. This environment may reduce antibiotic toxicity to the gut microbiome. Anaerobic bacteria use fermentative pathways to break down dietary fiber into viable carbon sources, producing SCFAs. SCFAs, particularly butyrate, are used by colonic epithelial cells as an energy source in an oxygen consuming reaction, helping to maintain barrier integrity and anaerobicity. Kim et al. (2018). The anaerobic environment of the colon allows for the proliferation of obligate anaerobes, which primarily use fermentation to produce energy. The anaerobic environment of the colon also promotes fermentation by facultative anaerobes via the “Pasteur effect”. This is seen in the metatranscriptomic data with the fiber-fed mice expressing decreased aerobic metabolism. See FIG. 3A-5D. compared to glucose-fed mice. Chemically, fermentation has negative enthalpy. Fermentation is considered a “cooling reaction” which can affect the redox potential (eH) of the chemical environment. Carlson et al. (2017). The redox potential of the gut can thermodynamically restrict metabolic activity with a low eH favoring fermentative metabolism.

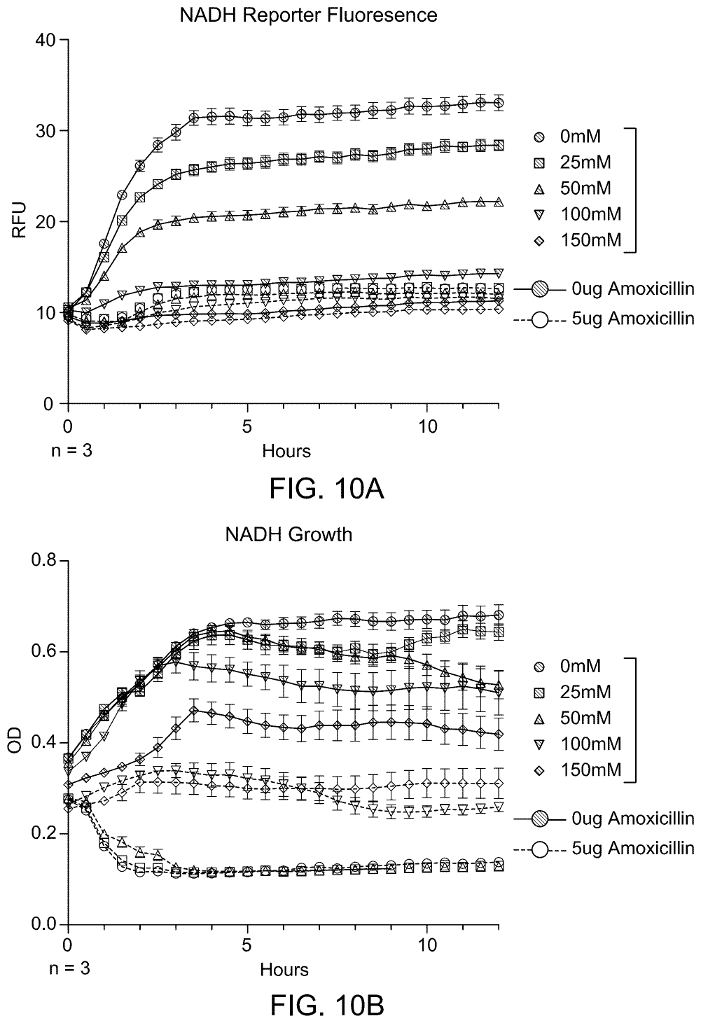

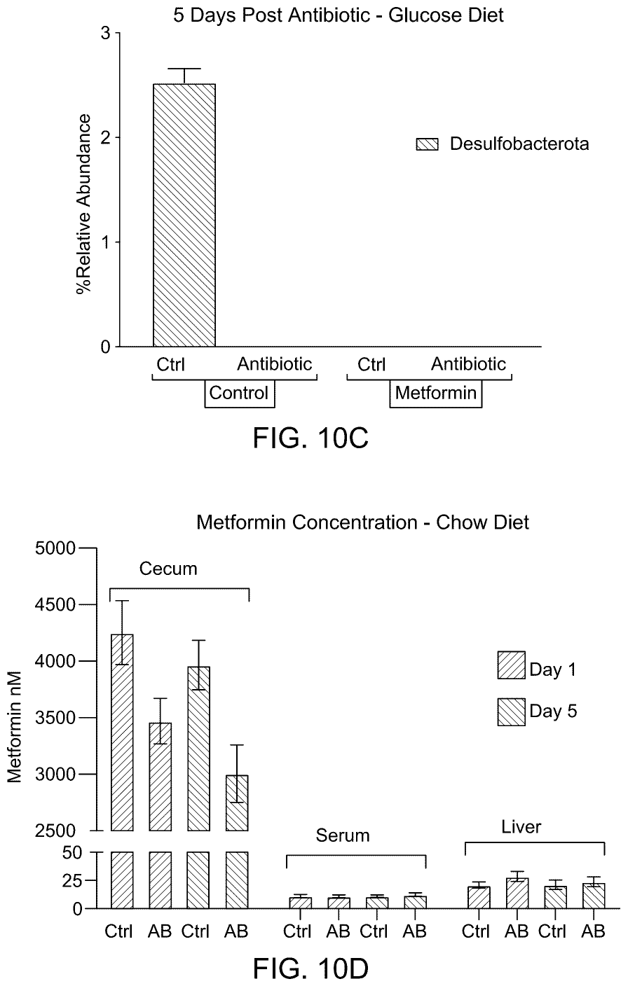

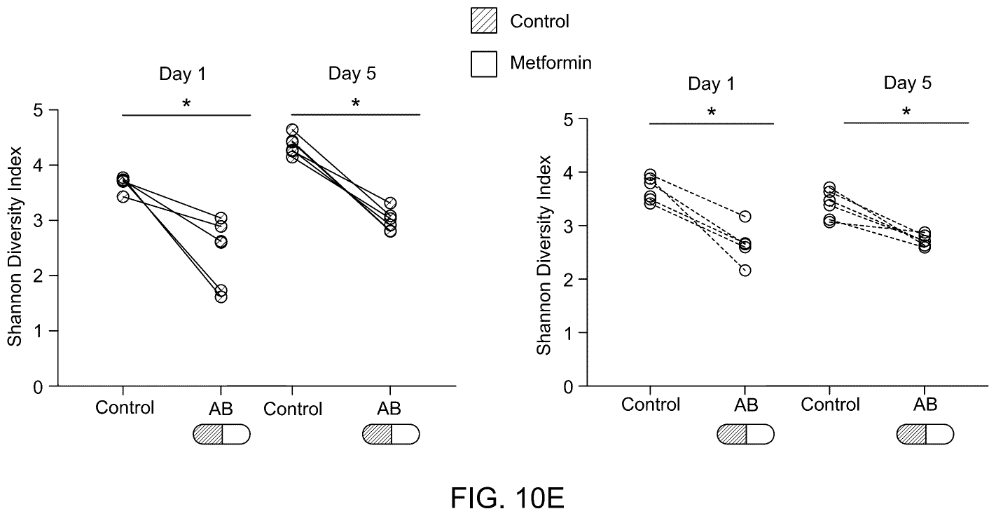

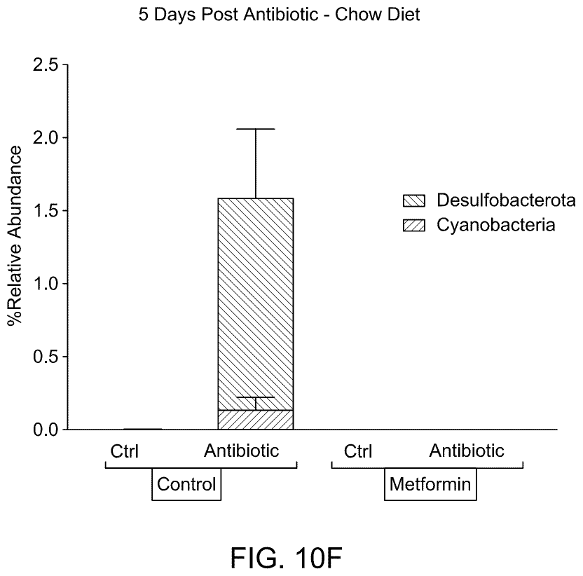

Consistent with the disruptive impact of antibiotics in the gut, under glucose conditions antibiotics increase the eH. See FIG. 10A-10F. A high eH environment (an oxidative environment) is associated with increased inflammation and aerobic bacteria. Chemically, a high eH environment selects for reactions with a high electric potential increasing the movement of electrons and oxidative stress. See Million & Raoult (2018). This oxidative environment has more free radicals such as ROS that can damage host tissue as they diffuse away from the gut.

Oxidative damage was postulated to affect the metabolism-driven mechanism of susceptibility. The high eH environment can contribute to elevated antibiotic disruption. See Dwyer et al. (2014). This could set up a negative feedback loop in which antibiotic damage leads to higher eH which further exacerbates the potential for AID.

The observed chemical and transcriptional impacts of diet and antibiotics are also translated into clear taxonomic changes associated with an inflammatory environment including more Proteobacteria and mucinphilic bacteria under glucose conditions. See FIG. 2A-2D. From an evolutionary perspective, these bacteria are phylogenetically similar oxygen-tolerant taxa. See Novakovsky et al. (2016); Spero et al. (2015). These microbes increased presence of electron transport proteins such as the NADH complex 1. Parey et al. (2020). The increase in measured eH is also associated with an increase in these complex 1 containing bacteria. See FIG. 5A-5D. Antibiotics shift the microbiome in a potentially harmful aerobic state through a chemical and taxonomic selection.

The use of fiber should be able to knock the community out of this negative state when given before, during or after antibiotic exposure. Besides acting as a nutrient for native gut microbes, fiber can be an electrochemical “insulator” by inhibiting flow of electrons to more reactive molecules due to its relative lack of free electrons. See Husson (2012).

### Example 10

### Metformin to Reduce Respiratory Metabolism and Antibiotic-Induced Gut Dysbiosis.

Fiber has a net protective effect during antibiotic treatment and recovery. See FIGS. 1A-1B and FIGS. 2A-2D. This effect could be explained by a shift in the metabolic use within the gut such as the reduction of respiratory activity. Conversely, this effect could result from electrochemical changes discussed in the EXAMPLES above or a combination of the two factors. To explore this dichotomy further, the inventors used the complex 1 inhibitor metformin in vitro and in vivo.

Metformin is the first line of treatment for diabetes. Metformin alters respiratory metabolism in all cells. Stynen et al. (2018). The chemistry of metformin shows a high affinity to iron. Many publications suggested that it behaves as an iron chelator disrupting iron-sulfur clusters such as those in bacterial electron transport chains. See Stynen et al. 2018). While in humans the metformin target is likely the bacterially derived mitochondria, metformin also disrupts respiratory activity in living bacteria. Li et al. (2020). The transcriptomic data showed that antibiotics lead to changes in respiratory activity with a particular induction of complex1, under glucose conditions. See FIG. 3A-5D. Inhibiting this respiratory burst with a pharmaceutical agent such as metformin should reduce the antibiotic toxicity in a manner similar to fiber.

The inventors observed that cultures of E. coli treated with metformin had reduced redox activity using a NADH/NAD+ ratiometric biosensor. See FIG. 6A-6F. The inventors also observed that metformin treatment reduced bacterial killing in E. coli and the gut commensal B. theta in vitro.

Mice fed a glucose-diet and metformin did not have an increase in Proteobacteria after twenty-four hours of antibiotic treatment. See FIG. 6A-6F. This positive outcome was not observed by Day 5 of antibiotic treatment. This result may show that the bacteria can recover from the respiratory inhibition with prolonged treatment. Metformin treatment led to a marked decrease in bacteria predominantly using anaerobic carbon fixation such as Desulfobacterota and Cyanobacteria. This effect was shown on the low-fiber glucose diet and the laboratory chow. See FIG. 6A-6F; FIG. 11A-11G. Because of the iron-chelating behavior of metformin, the drug may act as an inhibitor of the reverse Krebs cycle-carbon fixation- and the forward, in respiration. This shows that while metformin treatment prevents the initial increase in respiratory metabolism and resulting increase in inflammatory phyla, extended metformin treatment can lead to broader metabolic perturbations in the community that rely on electron transfers mediated by large iron-sulfur clusters. See Jordan et al. (2021). These iron-limiting conditions could lead to competition for iron driving the community composition by Day 5 of the assay. This is reflected in the data and the increases seen in bacteria seen by Day 5 of metformin treatment differ from the blooms seen post-antibiotic treatment with no metformin.

Metformin affects the outcome of antibiotics on the microbiome by modulating bacterial metabolism in the microbiome. Metformin also disrupts the host metabolism. The metformin concentration is higher than the typical in diabetic patients. As seen from the LC/MS data in FIG. 6A-6F, most of the metformin molecules remains in the gastrointestinal tract. Metformin is less likely to have toxicity to the host and more likely to affect the activity of bacteria in the gastrointestinal tract.

### Example 11

Short-Term Dietary Intervention with Whole Oats Protects from Antibiotic-Induced Dysbiosis.

In this EXAMPLE, the inventors used a multi-omics approach to characterize how a diet supplemented with oats, a rich source of microbiota-accessible carbohydrates, or dextrose impacts amoxicillin-induced changes to gut microbiome structure and transcriptional activity. Oat administration during amoxicillin challenge provides greater protection from AID than the always oats or recovery oats diet groups. The group in which oats were provided at the time of antibiotic exposure induced protection against AID. The other oat diets saw greater effects after amoxicillin challenge. The oat diets likewise reduced amoxicillin-driven elimination of Firmicutes compared to the dextrose diet. Gut communities fed dextrose were carbohydrate starved and favored respiratory metabolism and consequent metabolic stress management. Oat-fed communities shifted their transcriptomic profile and emphasized antibiotic stress management. The metabolic trends were exemplified when assessing transcriptional activity of the following two common gut commensal bacteria: Akkermansia muciniphila and Bacteroides thetaiotaomicron. These findings show that while host diet is important in shaping how antibiotics effect the gut microbiome composition and function, diet timing can have an even greater function in dietary intervention-based therapeutics.

The inventors used a multi-omics approach to show diets supplemented with oats, a rich source of microbiota-accessible carbohydrates, can confer protection against antibiotic-induced dysbiosis (AID). These findings affirm that not only is host diet important in shaping antibiotics effects on gut microbiome composition and function but also that the timing of these diets may have an even greater function in managing AID. This Example provides a nuanced perspective on dietary intervention against AID and may be informative on preventing AID during routine antibiotic treatment.

The inventors investigated whether a natural source of MACs, oats, could be leveraged temporally to manage AID in the murine gut microbiome. The inventors fed mice diets supplemented with dextrose or milled oats before, during, and after amoxicillin treatment. The impact of oats against murine gut microbiome AID was then assessed using metagenomics and metatranscriptomics. Feeding mice oats before, during, or after treatment with amoxicillin facilitated gut community recovery. Using oats “prophylactically,” during antibiotic treatment, dramatically mitigates murine gut microbiome AID. This attenuation may be attributed to transcriptional changes seen in the gut microbiome of the mice fed oats. This Example provides insight into how a tailored diet can be used alongside antibiotics to ameliorate off-target effects during treatment and potentially prevent incidental morbidities.

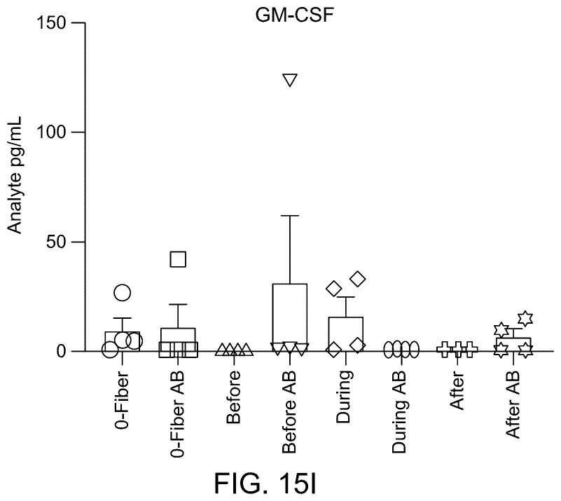

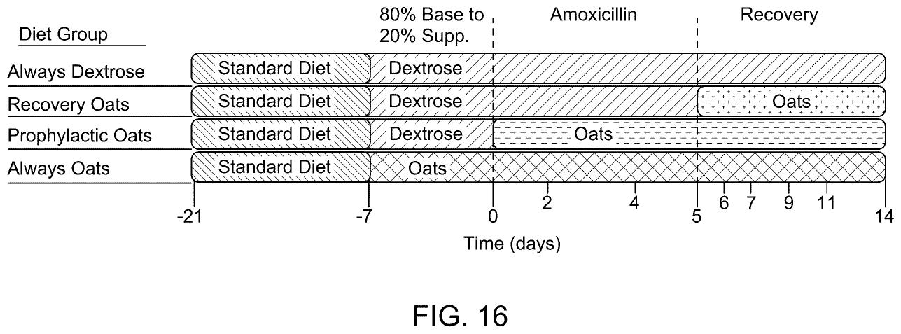

Oats protects taxonomic diversity during amoxicillin challenge and promote recovery. The inventors conducted the assay outlined in FIG. 16. Mice were randomly assigned into the following diet trials: dextrose (always dextrose), whole milled oats (always oats), and transitioning to an oats diet from dextrose during (prophylactic oats) or after (recovery oats) challenge with amoxicillin. Fecal pellet samples were collected throughout the assay, sequenced for 16S rRNA genes, and these sequences were then used to assess changes in diversity. Alpha diversity drops in each diet group from Day −7 to Day 0, reflecting the gut microbiome steady state transition from the habituation standard chow to the diets. The drop in dextrose diet alpha diversity is greater than that of the oats diet condition, reflecting the lower availability of polysaccharides in the dextrose diet. Alpha diversity significantly dropped in each group versus controls at Day 2 except for the prophylactic oats group. By conducting a simple MIC assay with Escherichia coli grown with and without oat supplementation, amoxicillin susceptibility was not directly affected by powdered oats.

This result shows that the attenuation seen in the host may not result from a direct blocking of drug activity.

Diversity continued to fall after Day 2 only in the always dextrose and recovery oats groups. The prophylactic oats group displayed a significant drop only on Day 5. Diversity recovery to Day 0 pre-amoxicillin challenge levels occurred as early as Day 9 for always dextrose, Day 11 for always oats, Day 6 for prophylactic oats, and Day 7 for recovery oats groups. Interestingly, prophylactic oats group diversity levels dropped significantly only on the final day of amoxicillin challenge.

These results show that transitioning from a simple carbohydrate diet to one high in natural MACs concurrent with or after antibiotic challenge mitigates murine gut microbiome perturbation and helps with taxonomical recovery.

Oat-dependent protection from AID depends on timing of supplementation. 16S rRNA sequences were used to derive group population structures. In the dextrose group, Verrucomicrobiota are the community's majority at Day 0 with Proteobacteria also present, consistent with past observations. See Cabral et al., Cell Metabolism 30:800-823 (2019) and Cabral et al., mSystems, 5, e00317-20 (2020). Firmicutes dominate the population composition in the oats group at Day 0. By Day 5, all challenged groups showed Verrucomicrobiota as the most abundant taxa, followed by Bacteroidota. At this time point, Firmicutes were the third most abundant phyla in the prophylactic oats and always oats groups. At this time point, Proteobacteria were the third in the always dextrose group. By Day 14, all the conditions stabilize toward a similar community composition. At this time point, the always oats control group differed, with Bacteroidota making up a larger relative share of its community.

To clarify amoxicillin-induced effects on phyla, statistical comparisons were drawn against each phylum within each group. Across each group on Day 5, the relative proportion of Bacteroidota receded and that of Verrucomicrobiota increased compared to controls. Proteobacteria remained unchanged in the always dextrose group but decreased in the always oats and prophylactic oats groups relative to controls. Firmicutes receded in the always dextrose and always oats diets but did not change significantly in the prophylactic oats group. Actinobacteria were present at low abundance in the always dextrose and prophylactic oats control groups. For finer taxonomical resolution, family level differential abundance analyses were drawn using DESeq2. Love, Huber, & Anders, Genome Biology, 15, 550 (2014). By Day 5, the always dextrose/recovery oats group had doubled the number of families decreasing at Day 2 before returning to pre-amoxicillin levels by Day 14. At Day 5, the majority of decreasing families were Firmicutes. The always oats group had a similar number of families decreasing on Day 2 as the always dextrose/recovery oats group that did not greatly increase by Day 5 before returning to pre-amoxicillin levels by day 14. Most of the decreasing families on Day 5 were Firmicutes. Strikingly, the prophylactic oats diet group had consistently fewer families decreasing on any given day.

These results show that gut communities provided simple carbohydrate diets are more susceptible to AID than those provided complex carbohydrate diets. These results also show that prophylactic supplementation of oats provides resistance to perturbation.

Oats mitigates Firmicutes reduction during amoxicillin treatment. The inventors conducted shotgun metagenomic sequencing of the always dextrose and prophylactic oats groups at the Day 5 terminal time point. The inventors used cecal material as opposed to fecal samples to generate a more reliable and timelier taxonomic and transcriptional profile and then classified reads against the Mouse Gastrointestinal Bacteria Catalogue (MGBC) database. See Beresford-Jones et al., Cell Host Microbe, 30, 124-138 (2022). Significantly changing species were identified using DESeq2 with results postprocessed as done by Cabral et al., mSystems, 5, e00317-20 (2020).

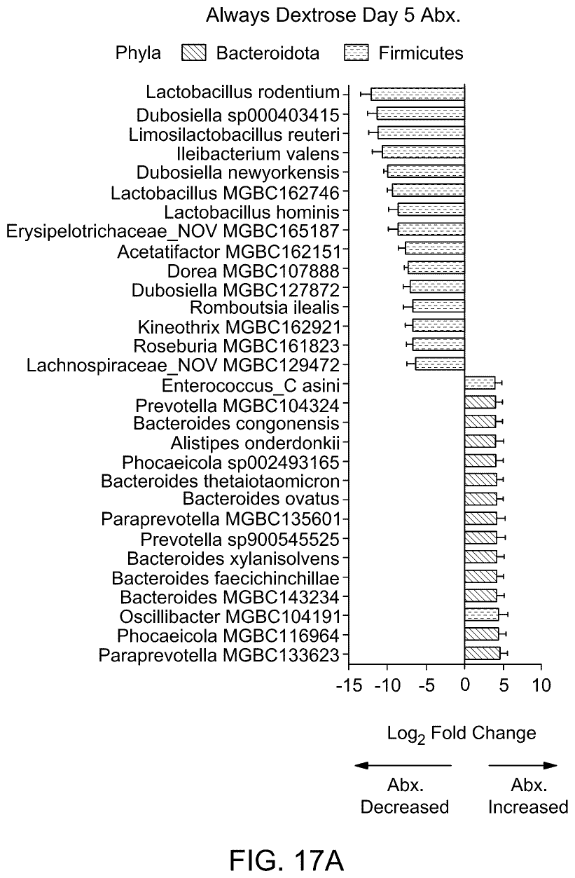

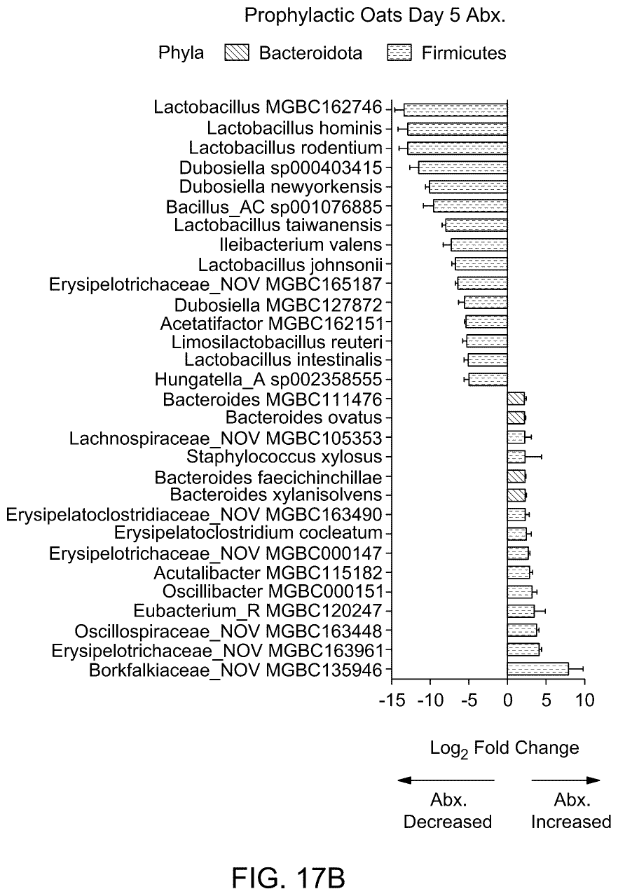

The always dextrose group had nearly three times as many species changing significantly than the prophylactic oats group. Most of the always dextrose group increasing species were Bacteroidota. Most of the decreasing species were Firmicutes. The prophylactic oats group saw significant increases and decreases in species belonging to the Firmicutes, but fewer were changing in total than in the always dextrose group. Most of decreasing species in either diet group were Firmicutes. Likewise, the majority of species increasing in the always dextrose group were Bacteroidota and in the prophylactic oats group were Firmicutes. Interestingly, Oscillospiraceae_NOV_MGBC163448, Eubacterium_R_MGBC120247, Acutalibacter_MGBC115182, Erysipelotrichaceae_NOV_MGBC000147, and Lachnospiraceae_NOV_MGBC105353 in FIG. 17A-17B were all species found to increase only in the prophylactic oats group. Erysipelotrichaceae_NOV_MGBC163961, was observed to increase in the prophylactic oats group but decrease in the always dextrose group. These results indicate fewer gut community members change significantly, especially Firmicutes members, when fed the oats diet versus the dextrose diet.

Prophylactic oats elicits a functional response from the murine gut microbiome. The inventors assessed how the two diet group gut communities respond functionally to amoxicillin challenge using cecal metatranscriptomics with classification against MetaCyc and a custom protein MGBC database. Average abundance patterns of the top pathways between the groups are reported in copies per million (CoPM).

Metagenomic pathways (DNA) between the two diet groups and their treatment conditions were comparable. Metatranscriptomic activity (RNA) of the amoxicillin-treated prophylactic oats group was nearly three times greater than amoxicillin-treated always dextrose group.

The inventors assessed how the diets change carbohydrate usage relative to amoxicillin challenge by aligning reads to the carbohydrate-active enzymes (CAZy) database. Cantarel et al., Nucleic Acids Research, 37, D233-D238 (2009). Most of the increasing carbohydrate-active enzymes in the always dextrose group involved mucus glycoprotein carbohydrate use and glucose acquisition from carbohydrate polymers. Most of the significantly decreasing carbohydrate-active enzymes related to complex polysaccharide use save for glycogen synthase (GT5). All the increasing prophylactic oats carbohydrate-active enzymes were involved in complex polysaccharide usage. Those enzymes decreasing significantly involved catabolism of simpler complex carbohydrate polymers or the acquisition and use of glycoprotein carbohydrates.

The inventors then investigated functional differences between the diet groups by aligning metatranscriptomic reads to the SEED subsystems database. Overbeek et al., Nucleic Acids Research, 42, D206-D214 (2014). The Always Dextrose group had more than three times the number of significantly changing subsystems than the prophylactic oats group. Of the subsystems increasing only in the always dextrose group, most related to anaerobic respiration, complex or simple carbohydrate sourcing, starch usage, the tricarboxylic acid (TCA) cycle, the pentose phosphate pathway (PPP), and oxidative stress resistance. Those decreasing involved complex carbohydrate usage, phosphotransferase system (PTS) simple sugar import, fermentation-related pathways, and glycogen synthesis, with subsystems increasing only in the prophylactic oats group related to anaerobic metabolism, oxidative or general stress, and antibiotic resistance pathways. Those decreasing involved NAD⁺ cofactor regeneration, the PhoB phosphate regulon, acetamido biosynthesis, and two PTS member's for either maltose/glucose or sucrose transport. These results suggest that the always dextrose gut communities emphasize carbohydrate sourcing, respiratory metabolism, and oxidative stress management. The prophylactic oats communities shift metabolism and favor generalized stress and antibiotic management.

The gut microbial community stress response profile is changed by oats. To further characterize the stress management profiles of the target diet groups, the inventors aligned metatranscriptomic reads to the Reference Sequence (RefSeq) database. O'Leary et al., Reference sequence (RefSeq) database at NCBI: current status, taxonomic expansion, and functional annotation. Nucleic Acids Research 44: D733-D745 (2016). The always dextrose group had greater than three times the number of total RefSeq features changing significantly than the prophylactic oats group. From these total pools, all stress resistance/management genes and features were identified. The increasing always dextrose group features involved oxidative stress resistance/management and general stress management with those decreasing largely related to general stress response features. The increasing prophylactic oats group features heavily skewed toward antibiotic resistance. Those decreasing were similar to the always dextrose group features except for additional oxidative stress resistance features.

The inventors next searched the feature list for electron transport chain proteins to gauge gut community commitment to respiratory energy production. Twenty-five such features changed significantly in the always dextrose group and nine in the prophylactic oats group.

The inventors then assessed the feature list to determine if markers of oxidative stress management were higher in the always dextrose versus prophylactic oats group. All four oxidative stress mitigation markers found were elevated higher in the always dextrose group than the prophylactic oats group.

These results show the always dextrose gut community is committed more to managing metabolic stress than managing antibiotic resistance like in the prophylactic oats community.

Oats alters Bacteroides thetaiotaomicron and Akkermansia muciniphila gene expression under amoxicillin challenge. The inventors assessed the transcriptional profiles of two abundant gut commensals: B. thetaiotaomicron and A. muciniphila. Species functional information derived from HUMAN3 metatranscriptomic outputs were analyzed by microbiome multivariable association with linear models (MaAsLin2) with the top thirty features by effect size. See Mallick et al., bioRxiv (2021).

All features involving glycolysis/gluconeogenesis, the TCA, the PPP, and simple and complex carbohydrate metabolism for the two species were then identified. In the top 30 B. thetaiotaomicron features, nearly half of the always dextrose condition features related to the targeted metabolic features. Fewer were seen in the prophylactic oats condition. Interestingly, lysozyme, known before improve composition and metabolic function of sow gut microbiota, was present only in the prophylactic oats group. See Zhou et al., Front MicroBiology, 10, 177 (2019). The enzyme β-N-acetylhexosaminidase, an enzyme that catalyzes the cleavage of β-N-acetylglucosamine residues from glycoproteins, was present only in the always dextrose group. See Ashida, Kato, & Yamamoto, Degradation of glycoproteins, In Kamerling H (ed), Comprehensive glycoscience (Elsevier, Oxford, United Kingdom 2007), p 151-170.

These results show a shift in response to diet and amoxicillin challenge that corroborates prior results from the full community analyses and provides further evidence for metabolic-driven AID tolerance.

Discussion In this EXAMPLE, the inventors used a multi-omics approach to show an oat diet, replete in MACs, can modulate the murine gut microbiome response to amoxicillin in a temporal fashion. Through 16S rRNA analysis, the inventors determined that phylum-level community structures changed comparably between the different diet groups throughout the assay. The changes to the overall prophylactic oats diet group community reflected far fewer actual microbial species changing than in any other diet group. The inventors observed a protective effect on Firmicutes, whose members are often among the first bacteria to use MACs, against amoxicillin in the prophylactic oat diet compared to that in the dextrose diet. See Flint et al., Gut Microbes, 3, 289-306 (2012). Proteobacteria are often cited clinically as human disease agents, Rizzatti et al., Biomed. Research Int., 2017, U.S. Pat. No. 9,351,507 (2017). Proteobacteria were found to significantly decrease in the oat diets after amoxicillin treatment but remained unchanged in the always dextrose diet.

The result of this Example shows that while oats can mitigate AID better than a diet comprised of dextrose, what mattered most was when the dietary transition to oats occurred. The metatranscriptomic results showed the prophylactic oats group leverages a different response to amoxicillin than the always dextrose group. The always dextrose diet group heavily emphasized carbohydrate scavenging, amino acid metabolism, and managing metabolic stress. The prophylactic oats diet group favored less energetic metabolic pathways and direct management of antibiotic resistance.

This observation affirms metabolic-driven antibiotic resistance but bolsters a second layer to the observed community resilience in the form of direct antibiotic resistance.

Defining the transcriptional profiles of the two abundant gut commensals, A. muciniphila and B. thetaiotaomicron, corroborated the metabolic observations and supported the idea of colonic mucus breakdown in fiber-deprived guts. Desai et al., Cell, 167, 1339-1353 (2016). A. muciniphila in the always dextrose diet group highly elevated glucosamine-6-phosphate deaminase and, especially, β-N-acetylhexosaminidase, suggesting mucus glycoprotein metabolism was favored in that diet group. The total elevated metabolic features of B. thetaiotaomicron in the always dextrose diet group indicates a broad emphasis on carbohydrate sourcing and use. The prophylactic oats diet group favored complex carbohydrates and linked metabolic pathways. These results taken together to show that the always dextrose group communities were trying to scavenge carbohydrates for respiratory metabolic processes. The prophylactic oats group preferred some respiratory and fermentative processes, consistent with previous work by Cabral et al., Cell Metabolism 30:800-823 (2019) and Schnizlein et al., mSphere 5:e00708-19 (2020). The result of this Example expands upon the idea that diets high in sugars or other host-accessible metabolites over MACs not only starves the gut microbiota of resources for proper gut community maintenance but also facilitates and exacerbates AID. Sonnenburg & Sonnenburg, Cell Metabolism 20:779-786 (2014).

Because both the always oats and prophylactic oats diet contained oats at the time of amoxicillin exposure, the transit time likely does not function in the difference between these conditions.

The metagenomic analysis in this Example was conducted on a consistent number of samples. The 16S analysis goal was to assess population scale shifts and not detect rare or marginally shifted taxa. The sample sizes were enough to identify such changes.

### OTHER EMBODIMENTS

Specific compositions and methods of the collagen microfiber scaffolds have been described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as persons skilled in the tissue engineering art will recognize. When the specification or claims recite method steps or functions in an order, alternative embodiments may perform the functions in a different order or substantially concurrently. The inventive subject matter should not be restricted except in the spirit of the disclosure.

When interpreting the disclosure, all terms should be interpreted in the broadest possible manner consistent with the context. Unless otherwise defined, all technical and scientific terms used in this specification have the same meaning as commonly understood by persons of ordinary skill in the tissue engineering art to which this invention belongs. This invention is not limited to the particular methodology, protocols, reagents, and the like described in this specification and can vary in practice. The terminology used in this specification is not intended to limit the scope of the invention, which is defined solely by the claims.

When a range of values is provided, each intervening value, to the tenth of the unit of the lower limit, unless the context dictates otherwise, between the upper and lower limit of that range and any other stated or intervening value in that range of values.

Some embodiments of the technology described can be defined according to the following numbered paragraphs:
1 Use of mitochondrial complex 1 inhibitors with fiber prebiotics to treat gut dysbiosis.
2. The use of embodiment 1, wherein the gut dysbiosis is antibiotic-induced gut dysbiosis (AID).
3. The use of embodiment 1, wherein the mitochondrial complex 1 inhibitor is selected from the group consisting of metformin, rotenone, piericidin A, benzamil, phenformin, and mito-metformin or other metformin analogs.
4. The use of embodiment 1, wherein the mitochondrial complex 1 inhibitor is an iron chelator (ferrous or ferric) which can modulate gastrointestinal redox.
5. The use of embodiment 4, wherein the iron chelator (ferrous or ferric) which can modulate gastrointestinal redox is selected from the group consisting of bathophenanthroline disulfonate and siderophores.
6. The use of embodiment 5, wherein the siderophore is selected from the group consisting of enterobactin, salmochelin, and mammalian “siderophores” such as 2,5-dihydroxybenzoic acid and lipocalin.
7. The method of embodiment 1, wherein the fiber prebiotics are plant-derived fibers are selected from the group consisting of pectin, inulin, dextrin, levan, arabinoxylan, beta glucan, cellulose, etc.
8. A method of dietary fiber supplementation to alleviate antibiotic-induced gut dysbiosis in a patient when supplemented before, during, or after antibiotic treatment, comprising the step of administering to the patent a diet of plant-derived fibers and mitochondrial complex 1 inhibitors.
9. A diagnostic method, comprising the steps: (a) sequencing 16s RNA genes or metagenomic genes from the microbiome from fecal samples to provide a baseline microbiome; and (b) identifying patients as being amenable to gut dysbiosis therapy when the patients have higher levels of respiratory bacteria or other complex 1 utilizing bacteria in the patient's gastrointestinal tract.
10. The diagnostic method of embodiment 9, wherein the sequencing 16s RNA genes or metagenomic genes from the microbiome from fecal samples to provide a baseline microbiome is performed the patients are administered the gut dysbiosis therapy.
11. The diagnostic method of embodiment 9, further comprising the therapeutic step of: (c) administering to the patent a diet of plant-derived fibers and mitochondrial complex 1 inhibitors.
12. The diagnostic and therapeutic method of embodiment 11, further comprising the step of: (d) when the patient experiences adverse gastrointestinal effects from the mitochondrial complex 1 inhibitor, the microbiome is reassessed to diagnosis which bacterial families are contributing to gastrointestinal distress.
13. The diagnostic and therapeutic method of embodiment 11, further comprising the step of: (d) assessing the efficacy of fiber supplementation by sequencing the microbiome and checking for abundance of inflammatory taxa such as Proteobacteria and other aerobic microbes; wherein a positive result is that the fiber supplementation decreases Proteobacteria/aerobic bacteria in the patients' gastrointestinal tract.
14. The diagnostic and therapeutic method of embodiment 11, further comprising the step of: (d) corroborating shifts in metabolic function induced by fiber supplementation that protect native gut microbes when challenged with antibiotics by chemical measurements of redox potential in the patient's gut and treatment with metformin.

### REFERENCES

Persons having ordinary skill in the molecular computing art can rely on the following patents, patent applications, scientific books, and scientific publications for enabling methods:

### Patent Citations

US20200345795A1 (Vitetta et al.). This patent application relates generally to methods for modulating the intestinal microbial profile and reducing intestinal dysbiosis in type 2 diabetic subjects or pre-diabetic subjects using metformin and a multi-strain probiotic combination. The disclosure also relates to methods for treating type 2 diabetes and metabolic complications and conditions associated therewith, and for delaying or inhibiting the onset of type 2 diabetes. In some embodiments, the methods may comprise administration to the subject of one or more probiotic components.
US20200376049A1 (Toledo et al.). This patent application provides pharmaceutical compositions comprising a plurality of purified viable microbes comprising at least one microbial entity classified as a gamma proteobacterium, and at least one prebiotic fiber. Also provided are methods for treating diabetes comprising administration of metformin and the microbial composition of any of the preceding claims.
US20200376049A1 (Toledo et al.). The patent application provides pharmaceutical compositions comprising a plurality of purified viable microbes comprising at least one microbial entity classified as a gamma proteobacterium, and at least one prebiotic fiber. Also provided are methods for treating diabetes comprising administration of metformin and the microbial composition.
WO2020257722A2 (Solarea Bio Inc.). A method for improving the efficacy of a probiotic microorganism in improving or maintaining health in a human subject. The probiotic assemblage may comprise a prebiotic polysaccharide or a prebiotic fiber.
CN106138114A (Jiang et al.). The patent application discloses a preparation method of a composite probiotic agent for preventing and treating diabetes and controlling body weight, in particular to a composite probiotic agent formed by compounding probiotics, probiotics and metformin and used for preventing and treating diabetes and controlling body weight and a preparation method of the composite probiotic agent.
JP2015518002A5 (Inovobiologic, Inc.). The formulation of metformin with other probiotics and drug adsorbents for extended release of the drug (glucomannan, guar gum, alginate) was created as an extended release formulation of metformin to slow host absorption and target diabetes or obesity in patients.
JP2021509661A (Performance Labs PTE, Ltd.). The patent application discloses fiber formulations to target gut microbiome dysbiosis including guar gum, citrus pectin, envelope fiber, gum arabic, and locust bean gum.

### Nonpatent Citations

Alagiakrishnan & Halverson, Holistic perspective of the role of gut microbes in diabetes mellitus and its management. World Journal of Diabetes, 12(9), 1463-1478 (Sep. 15, 2021).
Ashida, Kato, & Yamamoto, Degradation of glycoproteins, In Kamerling H (ed), Comprehensive glycoscience (Elsevier, Oxford, United Kingdom 2007), p 151-170.
Baltsavias et al., In vivo wireless sensors for gut microbiome redox monitoring. IEEE Trans. Biomed. Eng., 67, 1821-1830 (2020).
Beghini et al., Integrating taxonomic, functional, and strain-level profiling of diverse microbial communities with bioBakery 3. Elife, 10, e65088 (2021).
Beghini et al., Integrating taxonomic, functional, and strain-level profiling of diverse microbial communities with bioBakery 3. Elife, 10 (2021).
Belenky et al., Bactericidal antibiotics induce toxic metabolic perturbations that lead to cellular damage. Cell Reports, 13, 968-980 (2015).
Beresford-Jones et al., The Mouse Gastrointestinal Bacteria Catalogue enables translation between the mouse and human gut microbiotas via functional mapping. Cell Host Microbe, 30, 124-138 (2022).
Bharti & Grimm, Current challenges and best-practice protocols for microbiome analysis. Brief Bioinform., 22, 178-193 (2021).
Bokulich et al., Optimizing taxonomic classification of marker-gene amplicon sequences with QIIME 2's q2-feature-classifier plugin. Microbiome, 6, 90 (2018).
Bolger, Lohse, & Usadel, Trimmomatic: A flexible trimmer for Illumina sequence data. Bioinformatics, 30, 2114-2120 (2014).
Bolyen et al., Reproducible, interactive, scalable and extensible microbiome data science using QIIME 2. Nature Biotechnol. 37, 852-857 (2019). 10.1038/s41587-019-0209-9.
Buchfink, Xie, & Huson, Fast and sensitive protein alignment using DIAMOND. Nature Methods, 12, 59-60 (2015).
Cabral et al., Consumption of a Western-style diet modulates the response of the murine gut microbiome to ciprofloxacin. mSystems, 5, e00317-20 (2020).
Cabral et al., Microbial metabolism modulates antibiotic susceptibility within the murine gut microbiome. Cell Metabolism 30:800-823 (2019).
Callahan et al., DADA2: High-resolution sample inference from Illumina amplicon data. Nature Methods, 13, 581-583 (2016).
Cantarel et al., The Carbohydrate-Active EnZymes database (CAZy): an expert resource for glycogenomics. Nucleic Acids Research, 37, D233-D238 (2009).
Carlson et al., Probiotic dietary fiber and gut health: comparing the in vitro fermentations of beta-glucan, inulin and xylooligosaccharide. Nutrients, 9 (2017).
Caspi et al., The MetaCyc database of metabolic pathways and enzymes and the BioCyc collection of pathway/genome databases. Nucleic Acids Research, 38, D473-D479 (2010).
Chaudhari et al., Determination of metformin bio-distribution by LC-MS/MS in mice treated with a clinically relevant paradigm. PLOS One, 15, e0234571, (2020).
Costa et al., Short-term dietary intervention with whole oats protects from antibiotic-induced dysbiosis. Microbiology Spectrum, 11(4), e02376-23 (2023).
de Gunzburg et al., Protection of the human gut microbiome from antibiotics. J. Infect. Dis., 217, 628-636 (2018).
Deehan et al., Efficacy of metformin and fermentable fiber combination therapy in adolescents with severe obesity and insulin resistance: Study protocol for a double-blind randomized controlled trial. Trials, 22, 148 (2021).
Desai et al., A dietary fiber-deprived gut microbiota degrades the colonic mucus barrier and enhances pathogen susceptibility. Cell, 167, 1339-1353 (2016).
Dwyer et al., Antibiotics induce redox-related physiological alterations as part of their lethality. Proc. Natl. Acad. Sci. U.S.A., 111, E2100-2109 (2014).
Earle et al., Quantitative imaging of gut microbiota spatial organization. Cell Host Microbe 18, 478-488 (2015).
Fan & Pedersen, Gut microbiota in human metabolic health and disease. Nature Review Microbiol., 19, 55-71 (2021).
Flint et al., Microbial degradation of complex carbohydrates in the gut. Gut Microbes, 3, 289-306 (2012).
Fontaine, Metformin-induced Mitochondrial Complex I inhibition: Facts, uncertainties, and consequences. Front. Endocrinol. (Lausanne), 9, 753 (2018).
Furano, Luke, Howlett, & Campagnari, Identification of a conserved Moraxella catarrhalis haemoglobin-utilization protein, MhuA. Microbiology (Reading) 151, 1151-1158 (2005).
Hauser, Steinegger, & Söding, MMseqs software suite for fast and deep clustering and searching of large protein sequence sets. Bioinformatics, 32, 1323-1330 (2016).
Henson & Phalak, (Microbiota dysbiosis in inflammatory bowel diseases: in silico investigation of the oxygen hypothesis. BMC Syst Biol 11, 145 2017).
Hu & Rzymski, Non-photosynthetic Melainabacteria (Cyanobacteria) in human gut: Characteristics and association with health. Life (Basel) 12 (2022).
Husson et al., Practical improvements in soil redox potential (Eh) measurement for characterisation of soil properties. Application for comparison of conventional and conservation agriculture cropping systems. Anal. Chim, Acta, 906, 98-109 (2016).
Husson, Redox potential (Eh) and pH as drivers of soil/plant/microorganism systems: a transdisciplinary overview pointing to integrative opportunities for agronomy. Plant and Soil, 362, 389-417 (2012).
Hyatt et al., Prodigal: Prokaryotic gene recognition and translation initiation site identification. BMC Bioinformatics, 11, 119 (2010).
Jaroszewski, Mamun, & Czaja, Bidirectional interaction between tetracyclines and gut microbiome. Antibiotics 12(9), 1438 (2023).
Jones & Neish, Redox signaling mediated by the gut microbiota. Free Radic. Biol. Med., 105, 41-47 (2017).
Jordan et al., Spontaneous assembly of redox-active iron-sulfur clusters at low concentrations of cysteine. Nature Commun., 12, 5925 (2021).
Kang et al., MetaBAT 2: An adaptive binning algorithm for robust and efficient genome reconstruction from metagenome assemblies. PeerJ, 7, e7359 (2019).
Katoh, Misawa, Kuma, & Miyata, MAFFT: A novel method for rapid multiple sequence alignment based on fast Fourier transform. Nucleic Acids Research, 30, 3059-3066 (2002).
Khademian & Imlay, How microbes evolved to tolerate oxygen. Trends Microbiol., 29, 428-440 (2021).
Kim et al., Microbial metabolites, short-chain fatty acids, restrain tissue bacterial load, chronic inflammation, and associated cancer in the colon of mice. Eur. J. Immunol., 48, 1235-1247 (2018).
Korry, Cabral, & Belenky, Metatranscriptomics reveals antibiotic-induced resistance gene expression in the murine gut microbiota. Front. Microbiol., 11, 322 (2020).
Langmead & Salzberg, Fast gapped-read alignment with Bowtie 2. Nature Methods, 9, 357-359 (2012).
Lattimer & Haub, Effects of dietary fiber and its components on metabolic health. Nutrients, 2(12), 1266-1289 (2010). Dietary fiber is beneficial to the gut microbiome because it promotes native anaerobic gut microbes.
Lee et al., High-fat diet and antibiotics cooperatively impair mitochondrial bioenergetics to trigger dysbiosis that exacerbates pre-inflammatory bowel disease. Cell Host Microbe, 28, 273-284 e276 (2020).
Li et al., MEGAHIT: An ultra-fast single-node solution for large and complex metagenomics assembly via succinct de Bruijn graph. Bioinformatics, 31, 1674-1676 (2015). The publication reported the effects on individual bacterial strains of the gut microbiome.
Li et al., Metformin protects Escherichia coli from bleomycin-induced bactericide via enhanced generation of hydrogen peroxide. Free Radic, Res., 54(1), 64-75 (January 2020).
Litvak, Byndloss, & Baumler, Colonocyte metabolism shapes the gut microbiota. Science, 362 (2018).
Liu, Landick, & Raman, A regulatory NADH/NAD+ redox biosensor for bacteria. ACS Synth. Biol., 8, 264-273 (2019).
Lopatkin et al., Clinically relevant mutations in core metabolic genes confer antibiotic resistance. Science, 371 (2021).
Love, Huber, & Anders, Moderated estimation of fold change and dispersion for RNA-seq data with DESeq2. Genome Biology, 15, 550 (2014).
Lozupone & Knight, UniFrac: A new phylogenetic method for comparing microbial communities. Appl. Environ. MicroBiology, 71, 8228-8235 (2005).
Lozupone & Knight, UniFrac: a new phylogenetic method for comparing microbial communities. Appl. Environ. Microbiol., 71, 8228-8235 (2005).
Lu, Breitwieser, Thielen, & Salzberg, Bracken: Estimating species abundance in metagenomics data. PeerJ Comput., Sci 3, e104 (2017).
Mallick et al., Multivariable association discovery in population-scale meta-omics studies. bioRxiv (2021).
McIver et al., bioBakery: A meta'omic analysis environment. Bioinformatics, 34, 1235-1237 (2018).
McMurdie & Holmes, phyloseq: an R package for reproducible interactive analysis and graphics of microbiome census data. PLOS One, 8, e61217 (2013).
Miller, Liou, Lee, & Baumler, The longitudinal and cross-sectional heterogeneity of the intestinal microbiota. Curr. Opin. Microbiol., 63, 221-230 (2021).
Million & Raoult, Linking gut redox to human microbiome. Human Microbiome Journal 10, 27-32 (2018).
NCT04209075. Probiotics and metformin improve gut and hormones in type 2 diabetes in youth (MIGHTY-fiber). The clinical trial of fiber probiotics and metformin for combined effect on diabetes/obesity for the intended use on diabetic patients to alleviate gut side effects typically caused by metformin.
Ng et al., (Recovery of the gut microbiota after antibiotics depends on host diet, community context, and environmental reservoirs. Cell Host Microbe, 26, 650-665 e654 2019).
Novakovsky, Dibrova, & Mulkidjanian, Phylogenomic analysis of Type 1 NADH: quinone oxidoreductase. Biochemistry (Mosc) 81, 770-784 (2016).
Nurk, Meleshko, Korobeynikov, & Pevzner, (metaSPAdes: A new versatile metagenomic assembler. Genome Res., 27, 824-834 2017).
O'Leary et al., Reference sequence (RefSeq) database at NCBI: current status, taxonomic expansion, and functional annotation. Nucleic Acids Research 44: D733-D745 (2016).
Obrenovich, Siddiqui, McCloskey, & Reddy, The microbiota-gut-brain axis heart shunt Part I: The French paradox, heart disease and the microbiota. Microorganisms, 8 (2020).
Outpatient Antibiotic Prescriptions-United States, 2016. (n.d.).
Overbeek et al., The SEED and the rapid annotation of microbial genomes using subsystems technology (RAST). Nucleic Acids Research, 42, D206-D214 (2014).
Parada, Needham, & Fuhrman, Every base matters: assessing small subunit rRNA primers for marine microbiomes with mock communities, time series and global field samples. Environ. Microbiol. 18, 1403-1414 (2016).
Parey, Wirth, Vonck, & Zickermann, Respiratory complex I—structure, mechanism and evolution. Curr. Opin. Struct. Biol., 63, 1-9 (2020).
Patro et al., Salmon provides fast and bias-aware quantification of transcript expression. Nature Methods, 14, 417-419 (2017).
Penumutchu et al., Fiber supplementation protects from antibiotic-induced gut microbiome dysbiosis by modulating gut redox potential. Nature Communications, 14(1), 5161 (2023).
Pickard, Zeng, Caruso & Núñez, Gut microbiota: Role in pathogen colonization, immune responses, and inflammatory disease. Immunol. Rev., 279, 70-89 (2017).
Price, Dehal, & Arkin, FastTree 2—approximately maximum-likelihood trees for large alignments. PLOS One 5, e9490 (2010).
Pruesse et al., SILVA: A comprehensive online resource for quality checked and aligned ribosomal RNA sequence data compatible with ARB. Nucleic Acids Research, 35, 7188-7196 (2017).
Quast et al., The SILVA ribosomal RNA gene database project: Improved data processing and web-based tools. Nucleic Acids Research, 41, D590-D596 (2013).
Reese et al., Antibiotic-induced changes in the microbiota disrupt redox dynamics in the gut. Elife, 7 (2018).
Rena, Hardie, & Pearson, The mechanisms of action of metformin. Diabetologia, 60, 1577-1585 (September 2017). Metformin I was an antidiabetic/anti-obesogenic drug for the past sixty years.
Rigottier-Gois, Dysbiosis in inflammatory bowel diseases: The oxygen hypothesis. ISME J., 7, 1256-1261 (2013).
Rivera-Chávez, Lopez, & Bäumler, Oxygen as a driver of gut dysbiosis. Free Radical Biology and Medicine 105, 93-101 (2017).
Rizzatti et al., Proteobacteria: A common factor in human diseases. Biomed. Research Int., 2017, U.S. Pat. No. 9,351,507 (2017).
Rodriguez et al. Metformin, Current Opinion in Clinical Nutrition and Metabolic Care, 21 (4), 294-301 (July 2018). Metformin's therapeutic activity derives from its effects on the gut microbiome within the gastrointestinal (GI) tract with limited effects on other organs.
Rognes et al., VSEARCH: a versatile open source tool for metagenomics. PeerJ, 4, e2584 (2016).
Schnizlein et al., Dietary xanthan gum alters antibiotic efficacy against the murine gut microbiota and attenuates Clostridioides difficile colonization. mSphere, 5 (2020).
Segata et al., Metagenomic biomarker discovery and explanation. Genome Biology, 12, R60 (2011).
Silamikele et al., Metformin strongly affects gut microbiome composition in high-fat diet-induced type 2 diabetes mouse model of both sexes. Front. Endocrinol. (Lausanne) 12, 626359 (2021).
So et al., Dietary fiber intervention on gut microbiota composition in healthy adults: a systematic review and meta-analysis. Am. J. Clin. Nutr., 107, 965-983 (2018).
Sonnenburg & Sonnenburg, Starving our microbial self: the deleterious consequences of a diet deficient in microbiota-accessible carbohydrates. Cell Metabolism 20:779-786 (2014).
Speijer, Alternating terminal electron-acceptors at the basis of symbiogenesis: How oxygen ignited eukaryotic evolution. Bioessays, 39 (2017).
Spero, Aylward, Currie, & Donohue, Phylogenomic analysis and predicted physiological role of the proton translocating NADH:quinone oxidoreductase (complex I) across bacteria. mBio, 6(2), e00389-15 (Apr. 14, 2015). Phylogenomic analysis found that 50% of all bacteria on the planet have Complex 1 and these bacteria often exist in complex microbial ecosystems with bacteria lacking Complex 1.
Steffens et al., High CO₂ levels drive the TCA cycle backwards towards autotrophy. Nature, 592, 784-788 (2021).
Stokes, Lopatkin, Lobritz, & Collins, Bacterial metabolism and antibiotic efficacy. Cell Metab., 30, 251-259 (2019).
Stynen et al., Changes of cell biochemical states are revealed in protein homomeric complex dynamics. Cell 175, 1418-1429 e1419 (2018).
Suez et al., Post-antibiotic gut mucosal microbiome reconstitution is impaired by probiotics and improved by autologous FMT. Cell, 174(6), 1406-1423 e1416 (2018).
Suzek et al. UniRef: Comprehensive and non-redundant UniProt reference clusters. Bioinformatics, 23, 1282-1288 (2007).
Thompson et al., A communal catalogue reveals Earth's multiscale microbial diversity. Nature 551, 457-463 (2017).
Uritskiy, DiRuggiero, & Taylor, MetaWRAP-a flexible pipeline for genome-resolved metagenomic data analysis. Microbiome, 6, 158 (2018).
von Meijenfeldt et al., Robust taxonomic classification of uncharted microbial sequences and bins with CAT and BAT. Genome Biol., 20, 217 (2019).
Wastyk et al., Gut-microbiota-targeted diets modulate human immune status. Cell, 184, 4137-4153 e4114 (2021).
Westreich et al., SAMSA2: A standalone metatranscriptome analysis pipeline. BMC Bioinformatics, 19, 175 (2018).
Wood, Lu & Langmead, Improved metagenomic analysis with Kraken 2. Genome Biology, 20, 257 (2019).
Wu, Idemp (2017). https://github.com/yhwu/idemp.
Wu, Simmons, & Singer, MaxBin 2.0: An automated binning algorithm to recover genomes from multiple metagenomic datasets. Bioinformatics, 32, 605-607 (2016).
Wurster et al., Streptozotocin-induced hyperglycemia alters the cecal metabolome and exacerbates antibiotic-induced dysbiosis. Cell Reports, 37, 110113 (2021).
Yamamoto & Takai, Sulfur metabolisms in epsilon- and gamma-proteobacteria in deep-sea hydrothermal fields. Front. Microbiol., 2, 192 (2011).
Yang et al., Role of dietary nutrients in the modulation of gut microbiota: A narrative review. Nutrients, 12 (2020).
Youngblut & Ley, Struo2: Efficient metagenome profiling database construction for ever-expanding microbial genome datasets. PeerJ, 9, e12198 (2021).
Zake et al., Physiologically based metformin pharmacokinetics model of mice and scale-up to humans for the estimation of concentrations in various tissues. PLOS ONE 16(4), e0249594 (2021).
Zhang, Kobert, Flouri, & Stamatakis, PEAR: a fast and accurate Illumina Paired-End reAd mergeR. Bioinformatics 30, 614-620 (2014).
Zhou et al., Dietary lysozyme alters sow's gut microbiota, serum immunity and milk metabolite profile. Frontiers in MicroBiology, 10, 177 (2019).
Zheng et al., Probiotic mannan-oligosaccharides augment the hypoglycemic effects of metformin in correlation with modulating gut microbiota. J. Agric. Food Chem. 66(23): 5821-5831 (Jun. 13, 2018). Because the first line clinical medicine of metformin has several intestinal drawbacks, combination usage of metformin with a probiotic of konjac mannan-oligosaccharides (MOS) was implemented aiming to investigate whether there were intestinal synergetic effects and how MOS would function. The publication suggested that usage of MOS could augment the hypoglycemic effects of metformin in association with gut microbiota modulation, which could refer to further medication.

### Textbooks and Technical References

Current Protocols in Immunology (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
Current Protocols in Molecular Biology (CPMB), (2014). Frederick M. Ausubel (ed.), John Wiley and Sons (ISBN 047150338X, 9780471503385).
Current Protocols in Protein Science (CPPS), (2005). John E. Coligan (ed.), John Wiley and Sons, Inc.
Immunology (2006). Werner Luttmann, published by Elsevier.
Janeway's Immunobiology, (2014). Kenneth Murphy, Allan Mowat, Casey Weaver (eds.), Taylor & Francis Limited, (ISBN 0815345305, 9780815345305).
Laboratory Methods in Enzymology: DNA, (2013). Jon Lorsch (ed.) Elsevier (ISBN 0124199542).
Lewin's Genes XI, (2014). published by Jones & Bartlett Publishers (ISBN-1449659055).
Molecular Biology and Biotechnology: a Comprehensive Desk Reference, (1995). Robert A. Meyers (ed.), published by VCH Publishers, Inc. (ISBN 1-56081-569-8).
Molecular Cloning: A Laboratory Manual, 4th ed., Michael Richard Green and Joseph Sambrook, (2012). Cold Spring Harbor Laboratory Press, Cold Spring Harbor, N.Y., USA (ISBN 1936113414).
The Encyclopedia of Molecular Cell Biology and Molecular Medicine, Robert S. Porter et al., (eds.), published by Blackwell Science Ltd., 1999-2012 (ISBN 9783527600908).
The Merck Manual of Diagnosis and Therapy, 19th edition (Merck Sharp & Dohme Corp., 2018).
Pharmaceutical Sciences 23^{rd} edition (Elsevier, 2020).

All patents and publications cited throughout this specification are expressly incorporated by reference to disclose and describe the materials and methods that might be used with the technologies described in this specification. The publications discussed are provided solely for their disclosure before the filing date. They should not be construed as an admission that the inventors may not antedate such disclosure under prior invention or for any other reason. If there is an apparent discrepancy between a previous patent or publication and the description provided in this specification, the specification (including any definitions) and claims shall control. All statements as to the date or representation as to the contents of these documents are based on the information available to the applicants and constitute no admission as to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

1. A method of dietary fiber supplementation to alleviate antibiotic-induced gut dysbiosis in a patient when supplemented before, during, or after antibiotic treatment, comprising the step of administering to the patent a diet of plant-derived fibers and mitochondrial complex 1 inhibitors.

2. The method of claim 1, wherein the plant-derived fibers are selected from the group consisting of pectin, inulin, dextrin, levan, arabinoxylan, beta glucan, cellulose, etc.

3. The method of claim 1, wherein the mitochondrial complex 1 inhibitor is selected from the group consisting of metformin, rotenone, piericidin A, benzamil, phenformin, and mito-metformin or other metformin analogs.

4. The method of claim 1, wherein the mitochondrial complex 1 inhibitor is an iron chelator (ferrous or ferric) which can modulate gastrointestinal redox.

5. The method of claim 4, wherein the iron chelator (ferrous or ferric) which can modulate gastrointestinal redox is selected from the group consisting of bathophenanthroline disulfonate and siderophores.

6. The method of claim 4, wherein the siderophore is selected from the group consisting of enterobactin, salmochelin, and mammalian “siderophores” such as 2,5-dihydroxybenzoic acid and lipocalin.

7. A diagnostic method, comprising the steps:
(a) sequencing 16s RNA genes or metagenomic genes from the microbiome from fecal samples to provide a baseline microbiome; and
(b) identifying patients as being amenable to gut dysbiosis therapy when the patients have higher levels of respiratory bacteria or other complex 1 utilizing bacteria in the patient's gastrointestinal tract; and
(c) administering to the patent a diet of plant-derived fibers and mitochondrial complex 1 inhibitors.

8. The diagnostic method of claim 7, wherein the sequencing 16s RNA genes or metagenomic genes from the microbiome from fecal samples to provide a baseline microbiome is performed the patients are administered the gut dysbiosis therapy.

9. The diagnostic method of claim 7, further comprising the step of:
(d) when the patient experiences adverse gastrointestinal effects from the mitochondrial complex 1 inhibitor, the microbiome is reassessed to diagnosis which bacterial families are contributing to gastrointestinal distress.

10. The diagnostic method of claim 7, further comprising the step of:
(d) assessing the efficacy of fiber supplementation by sequencing the microbiome and checking for abundance of inflammatory taxa such as Proteobacteria and other aerobic microbes; wherein a positive result is that the fiber supplementation decreases Proteobacteria/aerobic bacteria in the patients' gastrointestinal tract.

11. The diagnostic method of claim 7, further comprising the step of:
(d) corroborating shifts in metabolic function induced by fiber supplementation that protect native gut microbes when challenged with antibiotics by chemical measurements of redox potential in the patient's gut and treatment with metformin.
